(19) United States
(12) Patent Application Publication
(10) Pub. No.: US 2008/0161232 A1
(43) Pub. Date: Jul. 3, 2008

(54) C5a Receptor Antagonist

(75) Inventors: Gerd Hummel, Berlin (DE); Elsa Locardi, Berlin (DE); Thomas Polakowski, Berlin (DE); Dirk Scharn, Berlin (DE); Karsten Schnatbaum, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: JERINI AG, Berlin (DE)

(21) Appl. No.: 11/814,050

(22) PCT Filed: Jan. 17, 2006
(86) PCT No.: PCT/EP06/00365

§ 371 Date: Jul. 16, 2007

(30) Foreign Application Priority Data
Jan. 17, 2005 (DE) 05000857.2

(51) Int. Cl. A61K 38/12; C07K 7/64; C07K 7/06; A61K 38/08; A61P 37/00; A61P 3/10

(52) U.S. Cl. 514/11; 530/321; 530/328; 514/16

(57) ABSTRACT

The present invention is related to a compound, preferably a C5a receptor antagonist, having the following structure: (I), whereby X1 is a radical having a mass of about 1-300, whereby X1 is preferably selected from the group comprising R5-, R5-CO—, R5-N(R6)-CO—, R5-O—CO—, R5-SO₂—, R5-N(R6)-SO₂—, R5-N(R6)-, R5-N(R6)-CS—, R5-N(R6)-C(NH)—, R5-CS—, R5-P(O)OH-5 R5-B(OH)—, and R5-CH═N—O—CH₂—CO—, whereby R5 and R6 are individually and independently selected from the group comprising H, F, hydroxy, alkyl, substituted alkyl, cycloalkyl, substituted cycloalkyl, heterocyclyl, substituted heterocyclyl, arylalkyl, substituted arylalkyl, aryl, substituted aryl, heteroaryl, substituted heteroaryl, acyl, substituted acyl, alkoxy, alkoxyalkyl, substituted alkoxyalkyl, aryloxyalkyl and substituted aryloxyalkyl, X2 is a radical that mimics the biological binding characteristics of a phenylalanine unit, X3 and X4 are individually and independently a spacer, whereby the spacer is preferably selected from the group comprising amino acids, amino acid analogs and amino acid derivates, X5 is a radical that mimics the biological binding characteristics of a cyclohexylalanine or homoleucine unit, X6 is a radical that mimics the biological binding characteristics of a tryptophane unit, X7 is a radical that mimics the biological binding characteristics of a norleucine or phenylalanine unit, a chemical bond X3 and X7 is formed, and the connecting lines—in formula (I) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds, whereby the compound is in particular useful for the manufacture of a medicament for the treatment of autoimmune diseases.

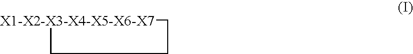

The present invention is related to antagonists of the C5a receptor and the use thereof.

Besides the adaptive immune system another—developmental much older—system for the defence against infection exists. This system is called complement system and consists of more than 30 soluble and membrane bound proteins. The complement system can be activated without or together with the adaptive immune system to eliminate, e.g., pathogenic bacteria. An uncontrolled activation or inadequate regulation of the complement system is related to a number of inflammatory diseases like septic shock, reperfusion injury, rheumatoid arthritis, transplant rejection, acute respiratory distress syndrome (ARDS), systemic lupus erythematosis (SLE), and glomerulonephritis. Numerous overviews over the relation between the complement system and diseases are published (e.g. Kirschfink 1997 Immunopharmacology 38: 51-62; Markides 1998 Pharmacological Reviews 50: 59-87, Walport 2001 The New England Journal of Medicine 344: 1140-1144, Walport 2001 The New England Journal of Medicine 344:1058-66).

Activation of the complement system takes place via three different pathways. They are called classical, alternative, and mannose-binding lectin (MBL) way. All pathways proceed via the sequential processing and thus activation of pro-forms of proteases. As each activated protease can cleave and therefore activate the next pro-form, an amplification of the initial reaction is obtained. This is similar to the clotting cascade. An overview over the complement system is given by Sim and Laich (2000 Biochemical Society Transactions 28: 545-550).

Some of the most important proteins that are generated upon complement activation are C3a, C3b, C5a, and C5b. These proteins will be discussed in more detail.

C3b is an essential part of a central protease of the complement cascade, the CS convertase. C3b is part of the CS convertase from both, the classical and alternative pathway of complement activation. The MLB pathway is proceeding via the convertases of the classical pathway, too. The CS convertase is responsible for the progress of the complement cascade and catalyses the cleavage of CS. Additionally, C3b is covalently attached to the surface of, e.g., bacteria which are thus more prone to phagocytosis by macrophages. Similar processes are described for immune complex clearance.

C3a is the smaller fragment that is produced in addition to C3b upon cleavage of C3. C3a is a comparatively weak chemokine and belongs to the anaphylatoxins.

C5b is formed upon cleavage of C5. This cleavage product is the starting point for the formation of the membrane attack complex (MAC). The MAC forms a pore which perforates both plasma membranes of bacteria and endogenous cells. Due to the pore formation the perforated cells can be lysed.

C5a is the 74 amino acid N-terminal cleavage product of the α-chain of plasma protein C5 and is released by the activity of the CS convertase. C5a is bound by its receptor which is referred to as C5a receptor C5aR1 or CD88, with high affinity and triggers a number of pro-inflammatory effects. It is one of the most potent chemokines and belongs similar to C3a to the anaphylatoxins. The C5aR can be found on many cells. This receptor is particularly found on neutrophils, macrophages, smooth muscle cells, and endothelial cells.

C5a release is thought to be directly or indirectly responsible for many diseases. Examples are sepsis (Huber-Lang et al. 2001 Faseb Journal 15: 568-570), multiple sclerosis (Mullerladner et al. 1996 Journal of Neurological Science 144: 135-141), reperfusion injury (Riley et al. 2000 Journal of Thoriacic and Cardiovascular Surgery 120: 350-358), psoriasis (Bergh et al. 1993 Archives of Dermatological Research 285: 131-134), rheumatoid arthritis (Woodruff et al. 2002 Arthritis and Rheumattism 46: 2476-85) und immune complex associated diseases in general (Heller et al. 1999 Journal of Immunology 163: 985-994). An overview over C5a related diseases is found in Köhl (2001 Molecular Immunology 38: 51-62).

Although it is obvious that C5a is responsible for many of the symptoms of inflammatory diseases, until today no drug directly aiming at the interaction between the receptor and its ligand was approved. The C5aR is a particularly interesting target. This is especially the case due to the finding that mice lacking the receptor do not show an unusual phenotype (Hopken et al. 1996 Nature 383: 86-89). This means that the complement cascade with its useful functions for defence against pathogens (MAC formation) and immune complex clearance can still proceed in an unhindered manner even when the receptor is completely inactivated.

The development of a specific C5a receptor antagonist also referred to herein as C5aR antagonist, was part of past programs. Among others, small molecules have been looked for. Examples for such molecules are L-156602 (Merck), RPR120033 (Rhone-Poulenc), W-54011 (Mitsubishi Pharma), and NGD 2000-1 (Neurogen). All currently known inhibitors with a molecular weight of <500 g/mol have at least one of the following drawbacks: low specificity, agonistic properties, too low affinity, poor solubility, inadequate metabolic stability, or inhibition of P450 enzymes.

Another approach for the development of C5aR inhibitors is based on the use of recombinant proteins. Examples for such protein based antagonists are CGS 32359 (Ciba-Geigy, Pellas et al. 1998 Journal of Immunology 160: 5616-5621), ΔpIII-A8 (Heller et al. 1999 Journal of Immunology 163: 985-994) and antibodies which can be of recombinant or non-recombinant origin (Huber-Lang et al. 2001 Faseb Journal 15: 568-570). These C5aR antagonists are proteins and therefore expensive in production. They have comparatively high affinities and specificities but have the drawback of pronounced immunogenicity. In addition, proteins can be effectively administered only by costly procedures such as, e.g., injection.

The C-terminal sequence information of C5a was used for the development of peptidic C5aR antagonists. Peptides as therapeutically useable antagonists of the C5aR are advantageous over protein therapeutics because of lower production costs, reduced immunogenicity, and high plasma stability. In addition they are more specific than most of the currently known small molecules. Many peptidic antagonists are described in the literature. A common feature of nearly all C5aR antagonists is their origin in the C-terminus of C5a. Examples for these peptidic C5aR antagonists or partial agonists are found in the following patents and patent applications: U.S. Pat. No. 4,692,511, U.S. Pat. No. 5,663,148, WO 90/09162, WO 92/11858, WO 92/12168, WO 92/21361, WO 94/07518, WO 94/07815, WO 95/25957, WO 96/06629, WO 99/00406 und WO 99/13899, WO 03/033528. In De Martino et al. (1995 Journal of Biological Chemistry 270: 15966-15969) a first attempt for a structural explanation of the importance of the C-terminal arginine in peptidic C5aR antagonists was made. It is shown on page 15967 that the C-terminal arginine is very important for the affinity and activity of the described peptides. It is pointed out that both the positively charged guanidinium group and the negative charge of the carboxy group are important for the affinity improving properties of arginine. The impact of both residues was further characterized (p. 15966), whereby guanidinium group is responsible for the energy releasing contact with the receptor, while the free carboxy group annuls the interference with Arg-206 of the receptor.

Nearly all of the C5aR binding peptides described to date have the positively charged amino acid arginine at the C-terminus. Sequences of these peptides are published both in scientific literature (Finch et al. 1999 Journal of Medicinical Chemistry 42: 1965-1974; Wong et al. 1999 IDrugs 2: 686-693; Psczkowski et al. 1999 Pharmacology 128: 1461-1466) and in the patent applications and patents recited above.

In WO 90/09162 38 peptidic inhibitors are presented along with their IC₅₀ values (example 2, 13, 23, 31, 91, 106, 111, 117, 131, 150, 165, 182, 188, 202, 213, 220, 229, 245, 247, 249, 279, 282, 295, 296, 305, 316, 338, 348, 377, 402, 404, 409, 421, 424, 432, 445, 455, 460). Out of these peptides 37 peptides have a C-terminal arginine and only one peptide has a different C-terminal amino acid (tyrosine, example 305). The amino acid sequence of example 305 of WO 90/09162 is Ac-Phe-Lys-Ala-Cha-Ala-Leu-ala-Tyr-OH and an IC₅₀ value of 0.17 μM was shown for the binding. This is more than a ten-fold decrease in the affinity compared to other described peptides with a C-terminal Arg (e.g. Ac-Phe-Lys-Ala-Cha-Ala-Leu-N-Methyl(D)ala-Arg-OH (example 296) and (N-Ethyl)Phe-Lys-Ala-Cha-Ala-Leu-N-Methyl(D)ala-Arg-OH (example 402) with an IC₅₀ value of 0.012 μM and 0.011 μM, respectively). In a functional assay as used in this application the tyrosine containing compound shows an IC50 value of 1.3 μM. Functional assays are generally more predictive for in vivo activities than binding assays. It becomes thus clear that the use of tyrosine as C-terminal amino acid did not lead to a peptide which could be used for the development of a pharmaceutically useable C5aR antagonist. This is possibly also the reason for the author not to describe further tyrosine containing peptides together with values for their activity.

In WO 92/12168 additional 20 peptides are described along with their IC₅₀ values (binding to C5aR). 19 out of these peptides have a terminal arginine which can be in either the D or the L form. One peptide has a C-terminal phenylbutanoyl residue which could interact via hydrophobic interactions. This peptide (example 170) has the sequence (N-Methyl)Phe-Lys-Pro-cha-Phe-Phenylbutanoyl and is said to have an IC50 value of only 2.6 μM which does not seem to be sufficient for use as a drug. An immediate comparison between the C-terminal argininyl and phenylbutanoyl from this application is not possible since a directly comparable structure was not disclosed. Example 105 of WO 92/12168 ((N-Methyl)Phe-Lys-Pro-cha-ψ55 CH₂—N(CH₂CH₂C₆H₅)}-Arg-OH) is the best suited compound for comparison with example 170. The IC₅₀ value for this hexamer is 0.36 μM. This means the substitution of Arg leads to an activity decrease in this example, too.

Among the 22 examples of WO 94/07518 for which IC₅₀ values have been presented, all peptides have a C-terminal arginine.

The IC₅₀ values indicated in WO 90/09162, WO 92/12168, and WO 94/07518 are derived from measurements with isolated membranes from polymorphonuclear neutrophilic granulocytes (PMN membranes) because at the time when these experiments were performed, C5a overexpressing cells could not be generated. Results from these measurements do not reflect the affinity of the compounds to whole cells. The compounds have a reduced affinity to receptors on whole cells (Kawai et al. 1991 Journal of Medicinal Chemistry 34: 2068-71; Rollins et al. 1988 Journal of Biological Chemistry 263: 520-526). It is, however, more meaningful to measure the biological activity rather than the binding of the antagonist to the receptor. Often such functional assays are used for G protein coupled receptors.

The examples presented in international patent applications WO 95/25957 und WO 96/06629 for which IC₅₀ values are known, are without any exception peptides containing a C-terminal arginine. This is also true for the papers of Wong et al. (Wong et al. 1998 Journal of Medicinal Chemistry 41: 3417-3425) and Finch et al. (Finch et al. 1999 Journal of Medicinal Chemistry 42: 1965-1974). In these papers 6 and 31, respectively, linear and cyclic 6 or 7-mer peptides are described.

In WO 99/00406 a number of cyclic and linear peptidic inhibitors are described. Their common feature is the C-terminal arginine. A model of the pharmacophore which is outlined in WO 99/00406, is directly pointing towards the required positive charge which can be realised by arginine (WO 99/00406 page 12, line 13ff).

The C-terminal arginine is also of crucial importance for the activity in the naturally occurring C5a. The agonistic potency is reduced by a factor of 10 to 1000, depending on the used assay system, when this arginine is cleaved off by carboxypeptidases (C5a-desArg) (Gerard und Gerard 1994 Annual Reviews in Immunology 12: 775-808).

In WO 03/033528 single substitutions of various amino acids in the molecule Ac-Phe[Orn-Pro-cha-Trp-Arg] (compound 1) are reported. A decrease of the affinity to the C5aR and a decrease in antagonistic potency is described for the substitution of the Arg with homoarginine (compound 44), citrulline (compound 45), lysine (Verbindung 47), or canavanine (compound 47). The reported IC₅₀ values as a measure for affinity are 1.36 μM (44), 6 μM (45), and 24 μM (47), respectively. No IC₅₀ value is reported for canavanine. This points to a significant decrease in the affinity to the C5a receptor due to these arginine substitutions (IC₅₀ of 1 is 0.45 μM). Apart from the effects of charged arginine substitutions (homoarginine and lysine), in particular the strong decrease in binding strength upon exchange of the charged arginine (0.45 μM) by the uncharged citrulline (6 μM) is remarkable. The antagonistic activity is reduced even more (Arg: 0.028 μM, Cit: 0.690 μM). The significance of a positive charge is thus underlined by the fact that the guanidinium group (Arg) and the urea group (Cit) are bioisosteres and need a comparable space. This also reflects that the size of the side chain itself is not sufficient as a criterion for predicting the activity. WO 03/033528 sets forth that the arginine (1) substitution to citrulline (45) results in a compound with allegedly remarkable antagonistic properties (p. 44, line 28 ff). However, the cut off rate for what is remarkable, is chosen arbitrarily and the significant 24-fold drop in activity underlines the in the prior art well known importance of the C-terminal arginine in the peptidic C5aR antagonists. The citrulline containing peptide 45 is by the way the only peptide that has no positive net charge under physiological conditions and for which a value for binding and the antagonistic activity is reported in WO 03/033528.

In a review of Morikis and Lambris (2002 Biochemical Society Transactions 30: 1026-1036) the importance of arginine for the affinity of agonists and antagonists to the C5a receptor is stressed.

It is apparent that the taching of the prior art requires a C-terminal localized positive charge for peptidic and peptidomimetic C5a ligands with noteworthy inhibitory activity (IC₅₀<200 nM). This charge is realized usually by arginine.

The problem underlying the present application is the provision of C5aR antagonists. Another problem underlying the present invention is the provision of drugs, that can be used for the treatment of diseases, in which the C5a receptor is involved in a causal, indirect or symptomatic manner.

In a first aspect of the invention the problem is solved by a compound, preferably a C5a receptor antagonist, having the following structure:

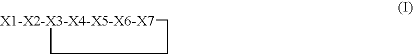

, whereby

X1 is a radical having a mass of about 1-300, whereby X1 is preferably selected from the group comprising R5-, R5-CO—, R5-N(R6)-CO—, R5-O—CO—, R5-SO₂—, R5-N(R6)-SO₂—, R5-N(R6)-, R5-N(R6)-CS—, R5-N(R6)-C(NH)—, R5-CS—, R5-P(O)OH—, R5-B(OH)—, R5-CH═N—O—CH₂—CO—, in which R5 and R6 are individually and independently selected from the group comprising H, F, hydroxy, alkyl, substituted alkyl, cycloalkyl, substituted cycloalkyl, heterocyclyl, substituted heterocyclyl, arylalkyl, substituted arylalkyl, aryl, substituted aryl, heteroaryl, substituted heteroaryl, acyl, substituted acyl, alkoxy, alkoxyalkyl, substituted alkoxyalkyl, aryloxyalkyl and substituted aryloxyalkyl,

X2 is a radical that mimics the biologic binding characteristics of a phenylalanine unit,

X3 and X4 individually and independently are a spacer, whereby the spacer is preferably selected from the group comprising amino acids, amino acid analogs and amino acid derivates,

X5 is a radical that mimics the biologic binding characteristics of a cyclohexylalanine or homoleucine unit,

X6 is a radical that mimics the biologic binding characteristics of a tryptophane unit,

X7 is a radical that mimics the biologic binding characteristics of a norleucine or phenylalanine unit,

a chemical bond between X3 and X7 is formed, and

the lines—in formula (I) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

In an embodiment X3 and X7 each being an amino acid, amino acid analog or amino acid derivative, whereby the chemical bond between X3 and X7 is formed under participation of at least one moiety of X3 and X7, and the moieties for X3 and X7 are individually and independently selected from the group comprising the C terminus, the N terminus and the respective side chain of the amino acid.

In an embodiment X1 is a radical having a mass of about 1-300, whereby X1 is preferably selected from the group comprising R5, R5-CO—, R5-N(R6)-CO—, R5-O—CO—, R5-SO₂—, R5-N(R6)-C(NH)—, whereby R5 and R6 are individually and independently selected from the group comprising H, alkyl, substituted alkyl, cycloalkyl, substituted cycloalkyl, heterocyclyl, substituted heterocyclyl, aryl and substituted aryl;

X2 and X6 are individually and independently an aromatic amino acid, a derivative or an analog thereof;

X5 and X7 are individually and independently a hydrophobic amino acid, a derivative or an analog thereof.

In an embodiment X2, X5, X6 and X7 individually and independently have the following structure:

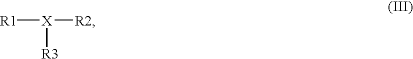

whereby

X is C(R4) or N,

R1 is optionally present and if R1 is present then R1 is a radical that is selected from the group comprising >N-R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, cycloalkylalkyl and substituted cycloalkylalkyl;

R2 is optionally present and if R2 is present then R2 is a radical, that is selected from the group comprising >C═O, >C═S, >SO₂, >S═O, >C═NH, >C═N—CN, >PO(OH), >B(OH), >CH₂, >CH₂CO, >CHF and >CF₂;

R4 is a radical, whereby the radical is selected from the group comprising H, F, CH₃, CF₃, alkyl and substituted alkyl;

the binding of structure (III) to the moieties of the molecule X1 and X3, X4 and X6, X5 and X7, and X6 and X3 is preferably carried out via R1 and R2;

for X2 and for X6 individually and independently R3 is a radical, whereby the radical comprises an aromatic group and is selected from the group comprising aryl, substituted aryl, heteroaryl, substituted heteroaryl, arylalkyl, substituted arylalkyl, heteroarylalkyl, substituted heteroarylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, allyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl; and

for X5 and for X7 individually and independently R3 is a radical, whereby the radical comprises an aliphatic or aromatic group and preferably is selected from the group comprising alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, heteroarylalkyl, substituted heteroarylalkyl, cycloalkylalkyl, substituted cycloalkylalkyl, heterocyclylalkyl, substituted heterocyclylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, allyloxy-heteroaryl, substituted allyloxy-heteroaryl, alkylthio-alkyl, substituted alkylthio-alkyl, alkylthio-cycloalkyl and substituted alkylthio-cycloalkyl.

In a preferred embodiment a ring is formed under participation of R3 and R4.

In an embodiment for X2 and for X6 individually and independently R3 is selected from the group comprising phenyl, substituted phenyl, benzyl, substituted benzyl, 1,1-diphenylmethyl, substituted 1,1-diphenylmethyl, naphthylmethyl, substituted naphthylmethyl, thienylmethyl, substituted thienylmethyl, benzothienylmethyl, substituted benzothienylmethyl, imidazolylmethyl, substituted imidazolylmethyl, indolylmethyl and substituted indolylmethyl.

In an embodiment for X5 and for X7 individually and independently R3 is selected from the group comprising C3-C5-alkyl, substituted C3-C5-alkyl, C5-C7-cycloalkyl, substituted C5-C7-cycloalkyl, C5-C7-cycloalkylmethyl, substituted C5-C7-cycloalkylmethyl, cycloalkylethyl, substituted cycloalkylethyl, benzyl, substituted benzyl, phenylethyl, naphthylmethyl, thienylmethyl, propenyl, propinyl, methylthioethyl, imidazolylmethyl, substituted imidazolylmethyl, indolylmethyl and substituted indolylmethyl.

In an embodiment X1 is selected from the group comprising H, acetyl, propanoyl, butanoyl, benzoyl, fluoromethylcarbonyl, difluoromethylcarbonyl, phenyl, oxycarbonyl, methyl-oxycarbonyl, phenyl-aminocarbonyl, methyl-aminocarbonyl, phenyl-sulfonyl, 2,6-dioxo-hexahydro-pyrimidine-4-carbonyl and methyl-sulfonyl.

In an embodiment X2 is a derivative of an amino acid that is selected from the group comprising phenylalanine, 2-fluoro-phenylalanine, 3-fluoro-phenylalanine, 4-fluoro-phenylalanine, 2-chloroophenylalanine, 3-chloroophenylalanine, 4-chlorophenylalanine, 1-naphthylalanine, 2-thienylalanine, 3-thienylalanine, 3,3-diphenylalanine, tyrosine, tryptophane, histidine and respective derivatives thereof;

or X2 and X1 taken together are PhCH₂CH₂CO— or PhCH₂—;

X6 is a derivative of an amino acid that is selected from the group comprising tryptophane, phenylalanine, tyrosine, histidine, 1-naphthylalanine, benzothienylalanine, 2-aminoindan-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof;

X5 is a derivative of an amino acid, that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof; and

X7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

In an embodiment X1 and/or X4 comprise one or more groups that improve water solubility, whereby the water solubility improving group is selected from the group comprising hydroxy, keto, carboxamido, ether, urea, carbamate, amino, substituted amino, guanidino, pyridyl and carboxyl.

In a second aspect of the invention the problem is solved by a compound, preferably a C5a receptor antagonist, having the following structure:

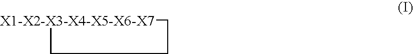

, whereby X1-X3 and X5-X7 are defined in accordance with the first aspect and whereby

X4 is a cyclic or a non-cyclic amino acid, whereby the cyclic amino acid is selected from the group comprising proline, pipecolinic acid, azetidin-2-carbonic acid, tetrahydroisochinoline-3-carbonic acid, tetrahydroisochinoline-1-carbonic acid, octahydroindole-2-carbonic acid, 1-aza-bicyclo-[3.3.0]-octane-2-carbonic acid, 4-phenyl-pyrrolidine-2-carbonic acid, cis-Hyp and trans-Hyp, and the non-cyclic amino acid is selected from the group comprising Ser, Gln, Asn, Cys(O₂CH₂CH₂CONH₂), Arg, Hyp(COCH₂OCH₂CH₂OCH₂CH₂OCH₃), Hyp(CONH—CH₂CH(OH)—CH₂OH) and respective derivatives thereof and respective analogs thereof; and

the lines—in formula (I) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

In an embodiment of the second aspect the amino acid represented by X4 is preferably selected from the group comprising proline, pipecolinic acid, azetidin-2-carbonic acid, tetrahydroisochinoline-3-carbonic acid, tetrahydroisochinoline-1-carbonic acid, octahydroindole-2-carbonic acid, 1-aza-bicyclo-[3.3.0]-octane-2-carbonic acid, 4-phenyl-pyrrolidine-2-carbonic acid, Hyp, Ser, Gln, Asn, Cys(O₂CH₂CH₂CONH₂) and Arg.

In an embodiment of the second aspect X2 is a derivative of an amino acid that is selected from the group comprising phenylalanine, 2-fluoro-phenylalanine, 3-fluoro-phenylalanine, 4-fluoro-phenylalanine, 2-chlorophenylalanine, 3-chlorophenylalanine, 4-chlorophenylalanine, 1-naphthylalanine, 2-thienylalanine, 3-thienylalanine, 3,3-diphenylalanine, tyrosine, tryptophane, histidine and respective derivatives thereof;

or X2 and X1 taken together are PhCH₂CH₂CO— or PhCH₂—;

X6 is a derivative of an amino acid that is selected from the group comprising tryptophane, phenylalanine, tyrosine, histidine, 1-naphthylalanine, benzothienylalanine, 2-aminoindan-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof;

X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof; and

X7 is a derivative of an amino acid that is selected from the group comprising norvaline, norleucine, homo-leucine, leucine, isoleucine, Valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

In a third aspect of the invention the problem is solved by a compound, preferably a C5a receptor antagonist, having the following structure:

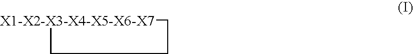

, whereby X1-X2 and X4-X7 are defined in accordance with the first and/or the second aspect of the present invention and whereby

X3 has the following structure

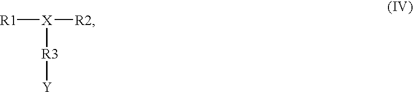

whereby

X is C(R4) or N,

R1 is optionally present and if R1 is present then R1 is a radical, which is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl;

R2 is optionally present and if R2 is present then R2 is a radical that is selected from the group comprising >C═O, >C—S, >SO₂, >PO(OH), >B(OH), >CH₂, >CH₂CO, >CHF and >CF₂;

R4 is a radical, whereby the radical is selected from the group comprising H, F, CF₃, alkyl and substituted alkyl;

the bond of structure (IV) to the moieties X2 and X4 preferably takes place via R1 and R2;

R3 is a radical, whereby the radical is selected from the group comprising H, alkyl, substituted alkyl, cycloalkyl, substituted cycloalkyl, heterocyclyl, substituted heterocyclyl, aryl, substituted aryl, heteroaryl, substituted heteroaryl, cycloalkylalkyl, substituted cycloalkylalkyl, heterocyclylalkyl, substituted heterocyclylalkyl, arylalkyl, substituted arylalkyl, heteroarylalkyl and substituted heteroarylalkyl;

Y is optionally present and if Y is present then Y is a radical that is selected from the group comprising —N(YB)—, —O—, —S—, —S—S—, —CO—, —C—N—O—, —CO—N(YB)— and

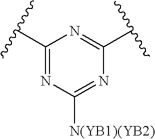

, whereby YB, YB1 and YB2 are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl.

In an embodiment of the third aspect R3 is a radical, whereby the radical is selected from the group comprising methyl, ethyl, propyl, butyl, benzyl and

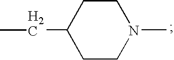

Y is optionally present and if Y is present then Y is a radical, whereby the radical is selected from the group comprising —N(YB)—, —O—, —S— and —S—S—, and YB is preferably defined as in the preceding embodiment.

In an embodiment of the third aspect X2 is a derivative of an amino acid that is selected from the group comprising phenylalanine, 2-fluoro-phenylalanine, 3-fluoro-phenylalanine, 4-fluoro-phenylalanine, 2-chlorophenylalanine, 3-chlorophenylalanine, 4-chlorophenylalanine, 1-naphthylalanine, 2-thienylalanine, 3-thienylalanine, 3,3-diphenylalanine, tyrosine, tryptophane, histidine and respective derivatives thereof;

or X2 and X1 taken together are PhCH₂CH₂CO— or PhCH₂—;

X6 is a derivative of an amino acid that is selected from the group comprising tryptophane, phenylalanine, tyrosine, histidine, 1-naphthylalanine, benzothienylalanine, 2-aminoindane-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof;

X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof; and

X7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

In an embodiment of any of the first to the third aspect of the present invention X3 is a derivative of an amino acid that is selected from the group comprising alpha-amino glycine, alpha-beta-diaminoproprionic acid (Dap), alpha-gamma-diaminobutyric acid (Dab), ornithine, lysine, homolysine, Phe(4-NH2), 2-amino-3-(4-piperidinyl)propionic acid and 2-amino-3-(3-piperidinyl)propionic acid, and the amino acid is modified at the side chain.

In a fourth aspect of the invention the problem is solved by a compound, preferably a C5a receptor antagonist, preferably according to any of the first to the third aspect of the present invention, having the following structure:

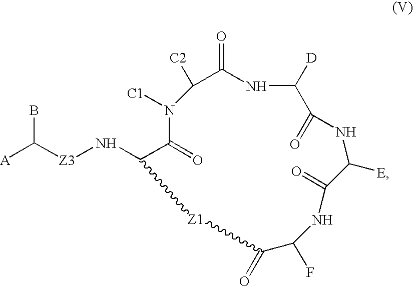

, whereby

A is selected from the group comprising H, NH2, NHalkyl, Nalkyl2, NHacyl and OH,

B is selected from the group comprising CH2(aryl), CH(aryl)₂, CH2(heteroaryl), substituted CH2(aryl), aryl, substituted aryl and heteroaryl,

C1 and C2 are individually and independently selected from the group comprising alkyl and substituted alkyl, whereby a bond can optionally be formed between C1 and C2,

D is selected from the group comprising alkyl, cycloalkyl, CH2(cycloalkyl), CH2CH2(cycloalkyl), CH2Ph(2-Me) and CH2-S-alkyl,

E is selected from the group comprising CH2(aryl), substituted CH2(aryl) and CH2(heteroaryl),

F is selected from the group comprising alkyl, CH2-S-alkyl, CH2CH2-S-Me, CH2CH═CH2, CH—CCH, cyclohexyl, CH2cyclohexyl, CH2Ph, CH2naphthyl, and CH2thienyl,

Z1 is selected from the group comprising (CH2)nNH with n=1, 2, 3, 4, (CH2)3O, (CH2)2O, (CH2)4, (CH2)3, CH2Ph(4-NH) and CH2(4-piperidinyl), and

Z3 is optionally present and if Z3 is present then Z3 is selected from the group comprising CO and CH2.

The individual moieties of this embodiment of the compound according to the present invention as depicted in formula (V), can be linked to the moieties of the compounds according to the present invention as of formula (I) as follows:

### X1-X2 is

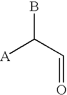

### X3 is

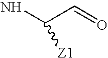

### X4 is

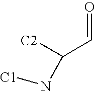

### X5 is

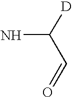

### X6 is

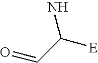

### and X7 is

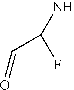

In an embodiment of the fourth aspect A is selected from the group comprising H, NH2, NHEt, NHAc, and OH,

B is selected from the group comprising CH2Ph, CH2Ph(4-F), CH(Ph)₂, CH2thienyl, CH2naphthyl, phenyl, Ph(4-F) and thienyl,

C1 is selected from the group comprising H and methyl, C2 is selected from the group comprising methyl and CH2OH, or if C1 and C2 are connected by a bond, the resulting structure is selected from the group comprising —(CH2)2-, —(CH2)3-, —(CH2)4- and —CH2CH(OH)CH2-.

D is selected from the group comprising CH2CH2iPr, CH2iPr, cyclohexyl, CH2cyclohexyl, CH2CH2cyclohexyl, CH2Ph(2-Me), CH2-S-tBu and CH2-S-iPr,

E is selected from the group comprising CH2Ph, CH2Ph(2-Cl), CH2Ph(3-Cl), CH2Ph(4-Cl), CH2Ph(2-F), CH2Ph(3-F), CH2Ph(4-F), CH2indolyl, CH2thienyl, CH2benzothienyl and CH2naphthyl,

F is selected from the group comprising (CH2)3CH3, (CH2)2CH3, (CH2)2-iPr, CH2-iPr, iPr, CH2-S-Et, CH2CH2-S-Me, CH2CH═CH2, CH2-CCH and cyclohexyl,

Z1 is selected from the group comprising (CH2)nNH with n=1, 2, 3, 4, (CH2)3O, CH2Ph(4-NH) and CH2(4-piperidinyl), and

Z3 is optionally present, and if Z3 is present, then Z3 is selected from the group comprising CO and CH2.

In a fifth aspect of the invention the problem is solved by a compound, preferably a C5a receptor antagonist, whereby the compound has the following structure:

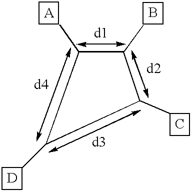

whereby d1, d2, d3 and d4 constitute the distances of A, B, C and D in at least one energetically accessible conformer of the compound and have the following values:

d1=5.1±1.0 Å

d2=11.5±1.0 Å

d3=10.0±1.5 Å

d4=6.9±1.5 Å

A and C are individually and independently a hydrophobic radical, whereby the hydrophobic radical is selected from the group comprising alkyl, cycloalkyl, heterocyclyl, aryl and heteroaryl;

B and D are individually and independently an aromatic or a heteroaromatic radical, whereby the aromatic radical preferably is aryl, and preferably the heteroaromatic radical is heteroaryl.

In an embodiment of the fifth aspect A and C are individually and independently selected from the group comprising C3-C6-alkyl, C5-C7-cycloalkyl, methylthioethyl, methylthio-tert-butyl, indolyl, phenyl, naphthyl, thienyl, propenyl, propinyl, hydroxyphenyl, indolyl and imidazolyl;

B is selected from the group comprising phenyl, substituted phenyl, naphthyl, thienyl, benzothienyl, hydroxyphenyl, indolyl, and imidazolyl; and

D is selected from the group comprising phenyl, naphthyl, thienyl, thiazolyl, furanyl, hydroxyphenyl, indolyl and imidazolyl.

In a sixth aspect of the invention the problem is solved by a compound, preferably a C5a receptor antagonist, having the following structure:

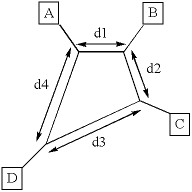

, whereby

A, B, C and D constitute the C-alpha atoms in amino acids, amino acid analogs or amino acid derivatives,

d1, d2, d3 and d4 constitute the distances of A, B, C and D in at least one energetically accessible conformer of the compound and have the following values:

d1=3.9±0,5 Å

d2=3.9±0,5 Å

d3=9.0±1,5 Å

d4=9.0±1,5 Å;

whereby the amino acids, whose alpha-atoms are constituted by A and C, individually and independently have a hydrophobic amino acid side chain that incorporates an alkyl-, cycloalkyl, cycloalkylalkyl, heterocyclyl, aryl, arylalkyl, heteroaryl, heteroarylalkyl or methylthio-tert-butyl group;

whereby the amino acids, whose alpha-atoms are constituted by B and D, individually and independently have an aromatic or heteroaromatic amino acid side chain that comprises an aryl, arylalkyl, heteroaryl or heteroarylalkyl group.

In an embodiment of the sixth aspect the amino acid, whose alpha-atom is constituted by A, is selected from the group comprising C3-C6-alkyl, methylthioethyl, propenyl, propinyl, R5, methyl-R5 and ethyl-R5, whereby R5 is a radical that is selected from the group comprising C5-C7-cycloalkyl, phenyl, substituted phenyl, hydroxyphenyl, indolyl, imidazolyl, naphthyl and thienyl;

whereby the amino acid whose alpha-atom is constituted by B, is selected from the group comprising R5, methyl-R5 and ethyl-R5, whereby R5 is a radical that is selected from the group comprising phenyl, substituted phenyl, naphthyl, thienyl, benzothienyl, hydroxyphenyl, indolyl and imidazolyl;

whereby the amino acid whose alpha-atom is constituted by C, is selected from the group comprising C3-C6-alkyl, R5, methyl-R5 and ethyl-R5, whereby R5 is a radical that is selected from the group comprising C5-C7-cycloalkyl, phenyl, 1-methyl-phenyl, 2-methyl-phenyl, 3-methyl-phenyl and S-tBu; and

whereby the amino acid whose alpha-atom is constituted by D, is selected from the group comprising R5, methyl-R5 and ethyl-R5, whereby R5 is a radical that is selected from the group comprising phenyl, naphthyl, thienyl, thiazolyl, furanyl, hydroxyphenyl, indolyl and imidazolyl.

In a seventh aspect of the invention the problem is solved by a compound, preferably a C5a receptor antagonist, having the following structure:

X1-X2-X3-X4-X5-X6-X7-X8 (II)

, whereby

X1 is a radical having a mass of about 1-300, whereby X1 is preferably selected from the group comprising R5-, R5-CO—, R5-N(R6)-CO—, R5-CO—, R5-SO₂—, R5-N(R6)-SO₂—, R5-N(R6)-, R5-N(R6)-CS—, R5-N(R6)-C(NH)—, R5-CS—, R5-P(O)OH—, R5-B(OH)—, R5-CH═N—O—CH₂—CO—, whereby R5 and R6 are individually and independently selected from the group comprising H, F, hydroxy, alkyl, substituted alkyl, cycloalkyl, substituted cycloalkyl, heterocyclyl, substituted heterocyclyl, arylalkyl, substituted arylalkyl, aryl, substituted aryl, heteroaryl, substituted heteroaryl, acyl, substituted acyl, alkoxy, alkoxyalkyl, substituted alkoxyalkyl, aryloxyalkyl and substituted aryloxyalkyl,

X2 is a radical that mimics the biological binding characteristics of a phenylalanine unit,

X3 and X4 are individually and independently a spacer, whereby the spacer is preferably selected from the group comprising amino acids, amino acid analogs and amino acid derivates,

X5 is a radical that mimics the biological binding characteristics of a cyclohexylalanine or homoleucine unit,

X6 is a radical that mimics the biological binding characteristics of a tryptophan unit,

X7 is a radical that mimics the biological binding characteristics of a norleucine or phenylalanine unit,

X8 is a radical which is optionally present in structure II, and if present it is selected from the group comprising H, NH₂, OH, NH—OH, NH-Oalkyl, amino, substituted amino, alkoxy, substituted alkoxy, hydrazino, substituted hydrazino, aminooxy, substituted aminooxy, alkyl, substituted alkyl, cycloalkyl, substituted cycloalkyl, heterocyclyl, substituted heterocyclyl, heteroaryl, substituted heteroaryl, arylalkyl, substituted arylalkyl, aryl, substituted aryl, amino acid, amino acid derivative and amino acid analogon;

the connecting lines—in formula (II) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

In an embodiment of the seventh aspect X1 is a radical having a mass of about 1-300, whereby the radical is preferably selected from the group comprising R5, R5-CO—, R5-N(R6)-CO—, R5-O—CO—, R5-SO₂—, R5-N(R6)-C(NH)—, whereby preferably R5 and R6 are individually and independently selected from the group comprising H, alkyl, substituted alkyl, cycloalkyl, substituted cycloalkyl, heterocyclyl, substituted heterocyclyl, aryl and substituted aryl;

X2 and X6 are individually and independently an aromatic amino acid, a derivative or an analogon thereof;

X5 and X7 are individually and independently a hydrophobic amino acid, a derivative or an analogon thereof.

In an embodiment of the seventh aspect X2, XS, X6 and X7 individually and independently have the following structure:

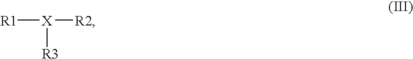

whereby

X is C(R4) or N,

R1 is optionally present and if R1 is present R1 is a radical, that is selected from the group comprising >N-R1B, >C(R1B)(R1D) and >O, whereby R1B and RID are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, cycloalkylalkyl and substituted cycloalkylalkyl;

R2 is optionally present and if R2 is present than R2 is a radical, that is selected from the group comprising >C═O, >C═S, >SO₂, >S═O, >C—NH, >C═N—CN, >PO(OH), >B(OH), >CH₂, >CH₂CO, >CHF and >CF₂;

R4 is a radical, whereby the radical is selected from the group comprising H, F, CH₃, CF₃, alkyl and substituted alkyl;

and the binding of structure (III) to the moieties X1 and X3, X4 and X6, X5 and X7, and X6 and X8 preferably takes place via R1 and R2;

for X2 and for X6 individually and independently R3 is a radical, whereby the radical comprises an aromatic group and is selected from the group comprising aryl, substituted aryl, heteroaryl, substituted heteroaryl, arylalkyl, substituted arylalkyl, heteroarylalkyl, substituted heteroarylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl; and

for X5 and for X7 individually and independently R3 is a radical, whereby the radical comprises an aliphatic or aromatic group and is preferably selected from the group comprising alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, heteroarylalkyl, substituted heteroarylalkyl, cycloalkylalkyl, substituted cycloalkylalkyl, heterocyclylalkyl, substituted heterocyclylalkyl, allyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl.

In an embodiment of the seventh aspect a ring is formed under participation of R3 and R4.

In an embodiment of the seventh aspect for X2 and for X6 individually and independently R3 is selected from the group comprising phenyl, substituted phenyl, benzyl, substituted benzyl, 1,1-diphenylmethyl, substituted 1,1-diphenylmethyl, naphthylmethyl, substituted naphthylmethyl, thienylmethyl, substituted thienylmethyl, benzothienylmethyl, substituted benzothienylmethyl, imidazolylmethyl, substituted imidazolylmethyl, indolylmethyl and substituted indolylmethyl.

In an embodiment of the seventh aspect for X5 and for X7 individually and independently R3 is selected from the group comprising C3-C5-alkyl, substituted C3-C5-alkyl, C5-C7-cycloalkyl, substituted C5-C7-cycloalkyl, C5-C7-cycloalkylmethyl, substituted C5-C7-cycloalkylmethyl, cycloalkylethyl, substituted cycloalkylethyl, benzyl, substituted benzyl, phenylethyl, naphthylmethyl, thienylmethyl, propenyl, propinyl, methylthioethyl, imidazolylmethyl, substituted imidazolylmethyl, indolylmethyl and substituted indolylmethyl.

In an embodiment of any of the preceding aspects and more particularly of the seventh aspect of the present invention X8 is selected from the group comprising H, OR1 and NR1R2, whereby R1 and R2 are individually and independently selected from the group comprising H, alkyl, aryl, cycloalkyl and arylalkyl.

In an embodiment of the seventh aspect X1 is selected from the group comprising H, acetyl, propanoyl, butanoyl, benzoyl, fluoromethylcarbonyl, difluoromethylcarbonyl, phenyl, oxycarbonyl, methyl-oxycarbonyl, phenyl-aminocarbonyl, methyl-aminocarbonyl, phenyl-sulfonyl, 2,6-dioxo-hexahydro-pyrimidine-4-carbonyl and methyl-sulfonyl.

In an embodiment of the seventh aspect X1 and/or X4 comprise one or more groups that improve water solubility, whereby the water solubility improving group is selected from the group comprising hydroxy, keto, carboxamido, ether, urea, carbamate, amino, substituted amino, guanidino, pyridyl and carboxyl.

In an eighth aspect of the invention the problem is solved by a compound, preferably a C5a receptor antagonist, having the following structure:

X1-X2-X3-X4-X5-X6-X7-X8 (II)

, whereby X1-X3 and X5-X8 are defined in accordance with the seventh aspect of the present invention and whereby

X4 is a cyclic or a non-cyclic amino acid, whereby the cyclic amino acid is selected from the group comprising proline, pipecolic acid, azetidine-2-carbonic acid, tetrahydroisoquinoline-3-carboxylic acid, tetrahydroisoquinoline-1-carboxylic acid, octahydroindole-2-carboxylic acid, 1-aza-bicyclo-[3.3.0]-octane-2-carboxylic acid, 4-phenyl-pyrrolidine-2-carboxylic acid, cis-Hyp and trans-Hyp, and the non-cyclic amino acid is selected from the group comprising Ser, Gln, Asn, Cys(O₂CH₂CH₂CONH₂), Arg, Hyp(COCH₂OCH₂CH₂OCH₂CH₂OCH₃), Hyp(CONH—CH₂CH(OH)—CH₂OH) and respective derivatives thereof and respective analogs thereof; and

the connecting lines—in formula (I) indicate chemical bonds, whereby the chemical bond individually and independently is preferably selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

In an embodiment of the eighth aspect of the present invention the amino acid represented by X4 is preferably selected from the group comprising proline, pipecolic acid, azetidine-2-carboxylic acid, tetrahydroisoquinoline-3-carboxylic acid, tetrahydroisoquinoline-1-carboxylic acid, octahydroindole-2-carboxylic acid, 1-aza-bicyclo-[3.3.0]-octane-2-carboxylic acid, 4-phenyl-pyrrolidine-2-carboxylic acid, Hyp, Ser, Gln, Asn, Cys(O₂CH₂CH₂CONH₂) and Arg.

In a ninth aspect of the invention the problem is solved by a compound, preferably a C5a receptor antagonist, having the following structure:

X1-X2-X3-X4-X5-X6-X7-X8 (II)

, whereby X1-X2 and X4-X8 are defined in accordance with the seventh and eighth aspect of the present invention and whereby

X3 has the following structure:

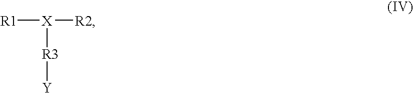

whereby

X is C(R4) or N,

R1 is optionally present and if R1 is present then R1 is a radical which is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl;

R2 is optionally present and if R2 is present then R2 is a radical which is selected from the group comprising >C═O, >C═S, >SO₂, >PO(OH), >B(OH), >CH₂, >CH₂CO, >CHF and >CF₂;

R4 is a radical, whereby the radical is selected from the group comprising H, F, CF₃, alkyl and substituted alkyl;

the binding of structure (IV) to the moieties X2 and X4 preferably takes place via R1 and R2;

R3 is a radical selected from the group comprising H, alkyl, substituted alkyl, cycloalkyl, substituted cycloalkyl, cycloalkylalkyl, substituted cycloalkylalkyl, heterocyclyl, substituted heterocyclyl, heterocyclylalkyl, substituted heterocyclylalkyl, aryl, substituted aryl, arylalkyl, substituted arylalkyl, heteroaryl, substituted heteroaryl, heteroarylalkyl, substituted heteroarylalkyl, acyl, substituted acyl, alkoxyalkyl, substituted alkoxyalkyl, aryloxyalkyl, substituted aryloxyalkyl, sulfhydrylalkyl, substituted sulfhydrylalkyl, hydroxyalkyl, substituted hydroxyalkyl, carboxyalkyl, substituted carboxyalkyl, carboxamidoalkyl, substituted carboxamidoalkyl, carboxyhydrazinoalkyl, ureidoalkyl aminoalkyl, substituted aminoalkyl, guanidinoalkyl and substituted guanidinoalkyl;

Y is optionally present and if Y is present then Y is a radical that is selected from the group comprising H, —N(YB1)-C0-YB2, —N(YB1)-CO—N(YB2)(YB3), —N(YB1)-C(N—YB2)-N(YB3)(YB4), —N(YB1)(YB2), —N(YB1)-SO₂—YB2, O—YB1, S—YB1, —CO—YB1, —CO—N(YB1)(YB2) and —C═N—O—YB1, whereby YB1, YB2, YB3 and YB4 are individually and independently selected from the group comprising H, CN, NO₂, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl.

In an embodiment of the ninth aspect R3 is a radical having the structure

—(CH₂)ₘ—Y (VII)

or

—(CH₂)ₘ—C6H₄—Y (VIII) ist

, whereby

m is 1, 2, 3 or 4;

Y is N(R3b)(R3c) or —N(YB1)-C(N—YB2)-N(YB3)(YB4), whereby R3b, R3c, YB1, YB2, YB3 and YB4 are individually and independently selected from the group comprising H, CN and alkyl.

In an embodiment of the ninth aspect a ring is formed between two moieties of the compound, whereby the moieties of the compound are individually and independently selected from the group comprising YB1, YB2, YB3 and YB4.

In an embodiment of the ninth aspect the ring is formed under participation of YB2 and YB3.

In an embodiment of the ninth aspect Y is

### —NH₂

or

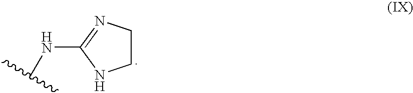

In an embodiment of any of the seventh to the ninth aspect of the present invention X2 is a derivative of an amino acid that is selected from the group comprising phenylalanine, 2-fluoro-phenylalanine, 3-fluoro-phenylalanine, 4-fluoro-phenylalanine, 2-chloro-phenylalanine, 3-chloro-phenylalanine, 4-chloro-phenylalanine, 1-naphthylalanine, 2-thienylalanine, 3-thienylalanine, 3,3-diphenylalanine, tyrosine, tryptophane, histidine and respective derivatives thereof;

or X2 and X1 together are PhCH₂CH₂CO— or PhCH₂—;

X6 is a derivative of an amino acid that is selected from the group comprising tryptophane, phenylalanine, tyrosine, histidine, 1-naphthylalanine, benzothienylalanine, 2-aminoindane-2-carboxylic acid, 2-thienylalanine, 3-thienylalanine, 2-fluoro-phenylalanine, 3-fluoro-phenylalanine, 4-fluoro-phenylalanine, 2-chloro-phenylalanine, 3-chloro-phenylalanine, 4-chloro-phenylalanine and respective derivatives thereof;

X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carboxylic acid, 2-methyl-D-phenylalanine and respective derivatives thereof; and 7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

In an embodiment of any of the aspects of the invention and preferably of the seventh to the ninth aspect of the present invention X3 is an amino acid derivative of an amino acid, whereby the amino acid is selected from the group comprising alpha-amino-glycine, alpha-beta-diaminopropionic acid (Dap), alpha-garnma-diaminobutanoic acid (Dab), ornithine, lysine, homolysine, Phe(4-NH₂), 2-amino-3-(4-piperidinyl)propionic acid and 2-amino-3-(3-piperidinyl)propionic acid, and the amino acid is derivatized at the side chain.

In a tenth aspect of the invention the problem is solved by a compound, preferably a C5a receptor antagonist, preferably a compound of any of the seventh to the ninth aspect, having the following structure:

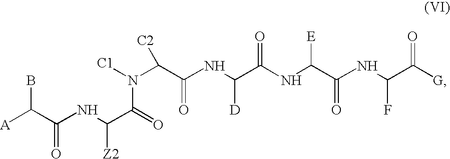

, whereby

A is selected from the group comprising H, NH₂, NHalkyl, Nalkyl₂, NHacyl, substituted NHacyl and OH,

B is selected from the group comprising CH₂(aryl), CH(aryl)₂, CH₂(heteroaryl) and substituted CH₂(aryl),

C1 and C2 are individually and independently selected from the group comprising alkyl and substituted alkyl, whereby optionally a bond can be formed between C1 and C2,

D is selected from the group comprising alkyl, cycloalkyl, CH₂(cycloalkyl), CH₂CH₂(cycloalkyl), CH₂Ph(2-Me) and CH₂—S-alkyl,

E is selected from the group comprising CH₂(aryl), substituted CH₂(aryl) and CH₂(heteroaryl),

F is selected from the group comprising alkyl, CH₂—S-alkyl, CH₂CH₂—S-Me, CH₂CH—CH₂, CH—CCH, cyclohexyl, CH₂cyclohexyl, CH₂Ph, CH₂naphthyl, and CH₂thienyl, and

Z2 is —R3-Y—, whereby R3 is selected from the group comprising H, alkyl, and arylalkyl, and Y is optionally present, and if Y is present, Y is selected from the group comprising H, N(YB1)(YB2), N(YB1)C(N—YB2)-N(YB3)(YB4),

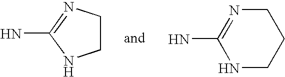

, whereby YB1, YB2, YB3 and YB4 are individually and independently selected from the group comprising H, CN and alkyl and optionally a ring is formed under participation of at least two of YB1, YB2, YB3 and YB4, and

G is selected from the group comprising H, OR1 and NR1R2, whereby R1 and R2 are individually and independently selected from the group comprising H, alkyl, aryl, cycloalkyl and arylalkyl.

In an embodiment of the tenth aspect A is selected from the group comprising H, NH₂, NHEt, NHAc, and OH,

B is selected from the group comprising CH₂Ph, CH₂Ph(4-F), CH(Ph)₂, CH₂thienyl and CH₂naphthyl,

C1 is selected from the group comprising H and methyl, C2 is selected from the group comprising methyl and CH₂OH, or if C1 and C2 are connected by a bond, the resulting structure is selected from the group comprising —(CH₂)₂—, —(CH₂)₃—, —(CH₂)₄— and —CH₂CH(OH)CH₂—.

D is selected from the group comprising CH₂CH₂iPr, CH₂iPr, cyclohexyl, CH₂cyclohexyl, CH₂CH₂cyclohexyl, CH₂Ph(2-Me), CH₂—S-tBu and CH₂—S-iPr,

E is selected from the group comprising CH₂Ph, CH₂Ph(2-Cl), CH₂Ph(3-Cl), CH₂Ph(4-Cl), CH₂Ph(2-F), CH₂Ph(3-F), CH₂Ph(4-F), CH₂indolyl, CH₂thienyl, CH₂benzothienyl and CH₂naphthyl,

F is selected from the group comprising (CH₂)₃CH₃, (CH₂)₂CH₃, (CH₂)₂-iPr, CH₂-iPr, iPr, CH₂—S-Et, CH₂CH₂—S-Me, CH₂CH═CH₂, CH₂—CCH and cyclohexyl,

Z2 is —R3-Y—, whereby R3 is selected from the group comprising CH₂, (CH₂)₂, (CH₂)₃, (CH₂)₄ and CH₂—C6H₄, and Y is selected from the group comprising NH₂, NHEt, N(Et)₂, NH—C(NH)—NH₂ and

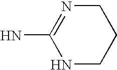

and

G is selected from the group comprising NH₂, NHMe, OH, and H.

In this aspect of the invention the moieties of the molecules shown in formula (VI) can be related to the parts of the molecules according to formula (II) in the following manner:

### X1-X2 is

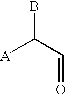

### X3 is

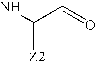

### X4 is

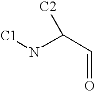

### X5 is

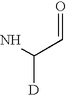

### X6 is

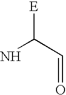

### X7 is

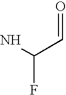

### and X8 is G.

In an embodiment of any of the first to the tenth aspect of the present invention the compound is one of the following compounds:

| No. | Compound |
|---|---|
| 1 | Ac-Phe-[Orn-Pro-cha-Trp-Phe] |
| 2 | Ac-Phe-[Orn-Hyp-cha-Trp-Phe] |
| 3 | HOCH₂(CHOH)₄—C═N—O—CH₂—CO-Phe-[Orn-Pro-cha-Trp- |
| | Nle] |
| 4 | X-Phe-[Orn-Pro-cha-Trp-Nle]; X = 2-acetamido-1-methyl- |
| | glucuronyl |
| 5 | Ac-Phe-[Orn-Hyp(COCH₂OCH₂CH₂OCH₂CH₂OCH₃)-cha- |
| | Trp-Nle] |
| 6 | Ac-Phe-[Orn-Hyp(CONH—CH₂CH(OH)—CH₂OH)-cha-Trp- |
| | Nle] |
| 20 | Ac-Phe-[Orn-Pro-cha-Trp-Ecr] |
| 28 | Ac-Phe-[Orn-Pro-cha-Trp-Nle] |
| 29 | Ac-Phe-[Orn-Pro-cha-Trp-Met] |
| 31 | Ac-Phe-[Orn-Pro-cha-Trp-Nva] |
| 32 | Ac-Phe-[Orn-Pro-cha-Trp-Hle] |
| 33 | Ac-Phe-[Orn-Pro-cha-Trp-Eaf] |
| 34 | Ac-Phe-[Orn-Pro-cha-Trp-Ebd] |
| 35 | Ac-Phe-[Orn-Pro-cha-Trp-Eag] |
| 36 | Ac-Phe-[Orn-Pro-cha-Trp-Pmf] |
| 37 | Ac-Phe-[Orn-Pro-cha-Trp-2Ni] |
| 38 | Ac-Phe-[Orn-Pro-cha-Trp-Thi] |
| 41 | Ph-CH₂—CH₂—CO-[Orn-Pro-cha-Trp-Nle] |
| 42 | H-Phe-[Orn-Pro-cha-Trp-Nle] |
| 43 | Ac-Lys-Phe-[Orn-Pro-cha-Trp-Nle] |
| 44 | H-Phe-[Orn-Ser-cha-Trp-Nle] |
| 51 | Ac-Phe-Orn-Pro-cha-Trp-Phe-NH₂ |
| 52 | Ac-Phe-Orn-Aze-cha-Bta-Phe-NH₂ |
| 53 | Ac-Phe-Orn-Pro-cha-Bta-2Ni—NH₂ |
| 54 | Ac-Phe-Orn-Pro-cha-Bta-Cha-NH₂ |
| 55 | Ac-Phe-Orn-Pip-cha-Trp-Phe-NH₂ |
| 56 | Ph-CH₂-[Orn-Pro-cha-Trp-Nle] |
| 57 | Ph-CH₂-[Orn-Pro-cha-Trp-Phe] |
| 58 | Ac-Phe-[Orn-Pro-cha-Trp-1Ni] |
| 59 | Ph-CH(OH)—CH₂—CO-[Orn-Pro-cha-Trp-Nle] |
| 61 | Ac-Phe-Orn-Pro-cha-Trp-Phe-NH₂ |
| 62 | Ac-Phe-Orn-Pro-cha-Bta-Phe-NH₂ |
| 64 | Ac-Phe-Orn-Pro-cha-Trp-2Ni—NH₂ |
| 65 | Ac-Phe-Orn-Pro-cha-Trp-Cha-NH₂ |
| 66 | Ac-Thi-Orn-Aze-cha-Bta-Phe-NH₂ |
| 67 | Ac-Thi-Orn-Pip-cha-Bta-Phe-NH₂ |
| 68 | Ac-Phe-Orn-Pro-cha-Trp-Eap-NH₂ |
| 69 | Me₂-Phe-Orn-Pro-cha-Trp-Phe-NH₂ |
| 70 | Ph₂-CH—CH₂—CO-Orn-Pro-cha-Trp-Phe-NH₂ |
| 71 | Ac-Ebw-Orn-Pro-cha-Trp-Phe-NH₂ |
| 72 | Ac-Phe-Orn-Pro-cha-Trp-NH—CH₂—CH₂-Ph |
| 73 | Ac-Phe-Orn-Aze-cha-Bta-NH—CH₂—CH₂-Ph |
| 74 | H-Phe-Orn-Pro-cha-Trp-Phe-NH₂ |
| 75 | H—Me-Phe-Orn-Pro-cha-Trp-Phe-NH₂ |
| 76 | Bu-NH—CO-Phe-Orn-Pro-cha-Trp-Phe-NH₂ |
| 77 | Ac-Thi-Orn-Pro-cha-Trp-Phe-NH₂ |
| 78 | Ac-Ebw-Orn-Pro-cha-Trp-Phe-NH₂ |
| 79 | Ac-Phe-Orn-Ala-cha-Trp-Phe-NH₂ |
| 80 | Ac-Phe-Orn-Pro-cha-Trp-Thi-NH₂ |
| 81 | Ac-Phe-Orn-Aze-cha-Pcf-Phe-NH₂ |
| 82 | Ac-Phe-Orn(Ac)-Pro-cha-Trp-Phe-NH₂ |
| 83 | Ac-Phe-Orn-Aze-cha-Trp-Phe-NH₂ |
| 84 | Ac-Phe-Trp-Pro-cha-Trp-Phe-NH₂ |
| 85 | Ph-NH—CO-Phe-Orn-Pro-cha-Trp-Phe-NH₂ |
| 86 | Bu-O—CO-Phe-Orn-Pro-cha-Trp-Phe-NH₂ |
| 87 | Ac-Phe-Lys-Pro-cha-Trp-Phe-NH₂ |
| 88 | Ac-Phe-Arg-Pro-cha-Trp-Phe-NH₂ |
| 89 | Ac-Phe-Gln-Pro-cha-Trp-Phe-NH₂ |
| 92 | Ac-Phe-Orn-Pip-cha-Trp-Phe-NH₂ |
| 93 | Ac-Phe-Orn-Hyp-cha-Trp-Phe-NH₂ |
| 94 | Ac-Phe-Orn-Pro-cha-Trp-1Ni—NH₂ |
| 95 | Ac-Phe-Orn-Aze-cha-Bta-Phe-NH—Me |
| 96 | CH₃—SO₂-Phe-Orn-Aze-cha-Bta-Phe-NH₂ |
| 99 | Ac-Phe-Orn-Aze-cha-Pff-Phe-NH₂ |
| 100 | Ac-Phe-Orn-Aze-cha-Mcf-Phe-NH₂ |
| 101 | Ac-Phe-Orn(Ac)-Aze-cha-Bta-Phe-NH₂ |
| 102 | Ac-Ebw-Orn-Pro-cha-Trp-Phe-NH₂ |
| 103 | Ac-Phe-Trp-Pro-cha-Trp-Phe-NH₂ |
| 104 | Ac-Phe-Arg-Pro-cha-Trp-Phe-NH₂ |
| 105 | Ac-Phe-Orn-Pip-cha-Trp-Phe-NH₂ |
| 106 | 3PP-Orn-Aze-cha-Bta-Phe-NH₂ |
| 107 | Ac-Phe-Orn-Tic-cha-Trp-Phe-NH₂ |
| 108 | Ac-Phe-Orn-Ser-cha-Trp-Phe-NH₂ |
| 109 | Ac-Phe-Orn-Pro-chg-Trp-Phe-NH₂ |
| 110 | Ac-Phe-Orn-Pro-hch-Trp-Phe-NH₂ |
| 111 | Ac-Phe-Orn-Pro-cha-Trp-Phg-NH₂ |
| 112 | Ac-Phe-Bta-Aze-cha-Bta-Phe-NH₂ |
| 113 | Ac-Phe-Trp-Pro-cha-Bta-Phe-NH₂ |
| 115 | Ac-Phe-Orn-Pip-cha-Trp-Phe-OH |
| 116 | Ac-Phe-Orn-Tic-cha-Trp-Phe-OH |
| 117 | Ac-Phe-Orn-Ser-cha-Trp-Phe-OH |
| 118 | Ac-Phe-Orn-Pro-chg-Trp-Phe-OH |
| 119 | Ac-Phe-Eec-Pro-cha-Bta-Phe-NH₂ |
| 120 | Ac-Phe-Nle-Pro-cha-Bta-Phe-NH₂ |
| 121 | Ac-Phe-Har-Pro-cha-Bta-Phe-NH₂ |
| 122 | Ac-Phe-Arg-Pro-cha-Bta-Phe-NH₂ |
| 123 | Ac-Phe-Cys(Acm)-Pro-cha-Bta-Phe-NH₂ |
| 124 | Ac-Phe-Mpa-Pro-cha-Bta-Phe-NH₂ |
| 125 | Ac-Eby-Orn-Pro-cha-Bta-Phe-NH₂ |
| 126 | Ac-Phg-Orn-Pro-cha-Bta-Phe-NH₂ |
| 127 | Ac-Phe-Paf-Pro-cha-Bta-Phe-NH₂ |
| 128 | H₂N—CO-Phe-Orn-Pro-cha-Bta-Phe-NH₂ |
| 129 | Me-O—CO-Phe-Orn-Pro-cha-Bta-Phe-NH₂ |
| 130 | (—CO—CH₂—NH—CO—)-Phe-Orn-Pro-cha-Bta-Phe-NH₂ |
| 132 | Ac-Phe-Orn-Pro-hch-Trp-Phe-OH |
| 133 | (—CO—CH₂—CH₂—CO—)-Phe-Orn-Pro-cha-Bta-Phe-NH₂ |
| 134 | ^{t}Bu-CO-Phe-Orn-Pro-cha-Bta-Phe-NH₂ |
| 135 | Ac-Lys-Phe-Orn-Aze-cha-Bta-Phe-NH₂ |
| 136 | Ac-Gly-Phe-Orn-Aze-cha-Bta-Phe-NH₂ |
| 137 | Ac-Arg-Phe-Orn-Aze-cha-Bta-Phe-NH₂ |
| 138 | Ac-His-Phe-Orn-Aze-cha-Bta-Phe-NH₂ |
| 139 | Ac-Ser-Phe-Orn-Aze-cha-Bta-Phe-NH₂ |
| 140 | Ac-Guf-Phe-Orn-Aze-cha-Bta-Phe-NH₂ |
| 141 | Ac-Dab-Phe-Orn-Aze-cha-Bta-Phe-NH₂ |
| 142 | FH₂C—CO-Phe-Orn-Pro-cha-Bta-Phe-NH₂ |
| 143 | Ac-Phe-Orn(Et₂)-Pro-cha-Trp-Phe-NH₂ |
| 144 | Ac-Phe-[Orn-Hyp-cha-Trp-Nle] |
| 145 | 3PP-[Orn-Hyp-cha-Trp-Nle] |
| 146 | Ac-Phe-[Orn-Pro-cha-Trp-Tyr] |
| 147 | Ac-Phe-[Orn-Pro-omf-Trp-Nle] |
| 149 | Ac-Phe-Orn-Pro-hle-Bta-Phe-NH₂ |
| 150 | Ac-Phe-Arg(CH₂—CH₂)-Pro-cha-Bta-Phe-NH₂ |
| 151 | Ac-Ala-Phe-Orn-Aze-cha-Bta-Phe-NH2 |
| 152 | Ac-Arg-Phe-Orn-Aze-cha-Bta-Phe-NH2 |
| 153 | Ac-Cit-Phe-Orn-Aze-cha-Bta-Phe-NH2 |
| 154 | Ac-Gly-Phe-Orn-Aze-cha-Bta-Phe-NH2 |
| 155 | Ac-Gly-Phe-Orn-Aze-chg-Bta-Phe-NH2 |
| 156 | Ac-Gly-Phe-Orn-Aze-hch-Bta-Phe-NH2 |
| 157 | Ac-Gly-Thi-Orn-Aze-cha-Bta-Phe-NH2 |
| 158 | Ac-His-Phe-Orn-Aze-cha-Bta-Phe-NH2 |
| 159 | Ac-Hyp-Phe-Orn-Aze-cha-Bta-Phe-NH2 |
| 160 | Ac-Lys-Phe-Orn-Aze-cha-Bta-Phe-NH2 |
| 161 | Ac-Mff-Orn-Pro-cha-Bta-Phe-NH2 |
| 162 | Ac-Mff-Orn-Pro-hle-Bta-Phe-NH2 |
| 163 | Ac-Mff-Orn-Pro-hle-Mcf-Mff-NH2 |
| 164 | Ac-Mmy-Orn-Pro-hle-Pff-Phe-NH2 |
| 165 | Ac-NMF-Orn-Pro-cha-Bta-Phe-NH2 |
| 166 | Ac-Off-Orn-Pro-cha-Bta-Phe-NH2 |
| 167 | Ac-Off-Orn-Pro-hle-Bta-Phe-NH2 |
| 168 | Ac-Orn-Phe-Orn-Aze-cha-Bta-Phe-NH2 |
| 169 | Ac-Pff-Orn-Pro-cha-Bta-Phe-NH2 |
| 170 | Ac-Pff-Orn-Pro-hle-Bta-Phe-NH2 |
| 171 | Ac-Pff-Orn-Pro-hle-Mcf-Pff-NH2 |
| 172 | Ac-Phe-[Cys-Pro-cha-Bta-Phe-Cys]-NH2 |
| 173 | Ac-Phe-[Orn-Asn-cha-Trp-Nle] |
| 174 | Ac-Phe-[Orn-Aze-cha-Trp-Nle] |
| 175 | Ac-Phe-[Orn-Chy-cha-Trp-Nle] |
| 176 | Ac-Phe-[Orn-HyA-cha-Trp-Phe] |
| 177 | Ac-Phe-[Orn-Hyp-hle-Bta-Phe] |
| 178 | Ac-Phe-[Orn-Hyp-hle-Mcf-Phe] |
| 179 | Ac-Phe-[Orn-Hyp-hle-Pff-Nle] |
| 180 | Ac-Phe-[Orn-Hyp-hle-Pff-Phe] |
| 181 | Ac-Phe-[Orn-Hyp-hle-Trp-Phe] |
| 182 | Ac-Phe-[Orn-Hyp-Mmf-Trp-Nle] |
| 183 | Ac-Phe-[Orn-Hyp-Mmf-Trp-Phe] |
| 184 | Ac-Phe-[Orn-NMD-cha-Trp-Nle] |
| 185 | Ac-Phe-[Orn-Pip-hle-Bta-Phe] |
| 186 | Ac-Phe-[Orn-Pro-cha-Pff-Nle] |
| 187 | Ac-Phe-[Orn-Pro-cha-Pff-Phe] |
| 188 | Ac-Phe-[Orn-Pro-cha-Trp-1Ni] |
| 189 | Ac-Phe-[Orn-Pro-cha-Trp-Cha] |
| 190 | Ac-Phe-[Orn-Pro-cha-Trp-Chg] |
| 192 | Ac-Phe-[Orn-Pro-cha-Trp-Ecr] |
| 193 | Ac-Phe-[Orn-Pro-cha-Trp-Leu] |
| 194 | Ac-Phe-[Orn-Pro-cha-Trp-nle] |
| 195 | Ac-Phe-[Orn-Pro-cha-Trp-Phe] |
| 196 | Ac-Phe-[Orn-Pro-hle-Bta-Nle] |
| 197 | Ac-Phe-[Orn-Pro-hle-Bta-Phe] |
| 198 | Ac-Phe-[Orn-Pro-hle-Pff-Phe] |
| 199 | Ac-Phe-[Orn-Pro-hle-Trp-Nle] |
| 200 | Ac-Phe-[Orn-Ser-cha-Trp-Nle] |
| 201 | Ac-Phe-[Orn-Ser-cha-Trp-Nle] |
| 202 | Ac-Phe-[Orn-Ser-hle-Trp-Nle] |
| 203 | Ac-Phe-[Orn-Thr-cha-Trp-Nle] |
| 204 | Ac-Phe-[Orn-Tic-cha-Trp-Nle] |
| 205 | Ac-Phe-[Orn-Tic-cha-Trp-Nle] |
| 206 | Ac-Phe-Ala-Pro-cha-Bta-Phe-NH2 |
| 207 | Ac-Phe-Arg-Pro-hle-Bta-Phe-NH2 |
| 208 | Ac-Phe-Arg-Pro-hle-Mcf-Phe-NH2 |
| 209 | Ac-Phe-Cit-Hyp-hle-Bta-Phe-NH2 |
| 210 | Ac-Phe-Cit-Pro-cha-Bta-Phe-NH2 |
| 211 | Ac-Phe-Cit-Pro-hle-Bta-Phe-NH2 |
| 212 | Ac-Phe-Cit-Ser-hle-Bta-Phe-NH2 |
| 213 | Ac-Phe-Dab-Aze-cha-Bta-Phe-NH2 |
| 214 | Ac-Phe-Dab-Aze-hle-Bta-Phe-NH2 |
| 215 | Ac-Phe-Dab-Pro-cha-Bta-Phe-NH2 |
| 216 | Ac-Phe-Dap-Pro-cha-Bta-Phe-NH2 |
| 217 | Ac-Phe-Ech-Pro-cha-Bta-Phe-NH2 |
| 218 | Ac-Phe-Eep-Pro-cha-Bta-Phe-NH2 |
| 219 | Ac-Phe-Fcn-Aze-cha-Bta-Phe-NH2 |
| 220 | Ac-Phe-Fcn-Pro-cha-Bta-Phe-NH2 |
| 221 | Ac-Phe-Fco-Pro-cha-Bta-Phe-NH2 |
| 222 | Ac-Phe-Fco-Pro-cha-Bta-Phe-NH2 |
| 223 | Ac-Phe-Fcp-Aze-cha-Bta-Phe-NH2 |
| 224 | Ac-Phe-Ffa-Aze-cha-Bta-Phe-NH2 |
| 225 | Ac-Phe-Ffa-Pro-cha-Bta-Phe-NH2 |
| 226 | Ac-Phe-Ffa-Pro-hle-Bta-Phe-NH2 |
| 227 | Ac-Phe-G23-Pro-cha-Bta-Phe-NH2 |
| 228 | Ac-Phe-Guf-Pro-cha-Bta-Phe-NH2 |
| 229 | Ac-Phe-Har-Aze-cha-Bta-Phe-NH2 |
| 230 | Ac-Phe-His-Pro-cha-Bta-Phe-NH2 |
| 231 | Ac-Phe-L22-Pro-cha-Bta-Phe-NH2 |
| 232 | Ac-Phe-OrA-Pro-cha-Bta-Phe-NH2 |
| 233 | Ac-Phe-OrE-Pro-cha-Bta-Phe-NH2 |
| 234 | Ac-Phe-Orn-Aze-hle-Bta-Phe-NH2 |
| 235 | Ac-Phe-Orn-Chy-cha-Bta-Phe-NH2 |
| 236 | Ac-Phe-Orn-Chy-hle-Pff-Phe-NH2 |
| 237 | Ac-Phe-Orn-G24-cha-Bta-Phe-NH2 |
| 238 | Ac-Phe-Orn-G25-cha-Bta-Phe-NH2 |
| 239 | Ac-Phe-Orn-G26-cha-Bta-Phe-NH2 |
| 240 | Ac-Phe-Orn-G27-cha-Bta-Phe-NH2 |
| 241 | Ac-Phe-Orn-G30-cha-Bta-Phe-NH2 |
| 242 | Ac-Phe-Orn-G31-cha-Bta-Phe-NH2 |
| 243 | Ac-Phe-Orn-Hse-cha-Bta-Phe-NH2 |
| 244 | Ac-Phe-Orn-Hyp-hle-Bta-Phe-NH2 |
| 245 | Ac-Phe-Orn-Hyp-hle-Pff-Phe-NH2 |
| 246 | Ac-Phe-Orn-NMA-cha-Bta-Phe-NH2 |
| 247 | Ac-Phe-Orn-NMS-cha-Bta-Phe-NH2 |
| 248 | Ac-Phe-Orn-Pro-cha-1Ni-Phe-NH2 |
| 249 | Ac-Phe-Orn-Pro-cha-Bta-1Ni—NH2 |
| 250 | Ac-Phe-Orn-Pro-cha-Bta-Bhf-NH2 |
| 251 | Ac-Phe-Orn-Pro-cha-Bta-Dff-NH2 |
| 252 | Ac-Phe-Orn-Pro-cha-Bta-Eaa-NH2 |
| 253 | Ac-Phe-Orn-Pro-cha-Bta-L19 |
| 254 | Ac-Phe-Orn-Pro-cha-Bta-Mcf-NH2 |
| 255 | Ac-Phe-Orn-Pro-cha-Bta-Mff-NH2 |
| 256 | Ac-Phe-Orn-Pro-cha-Bta-NH—CH(CH2OH)—CH2-Ph |
| 257 | Ac-Phe-Orn-Pro-Cha-Bta-NH-NBn-CO—NH2 |
| 258 | Ac-Phe-Orn-Pro-cha-Bta-Opa-NH2 |
| 259 | Ac-Phe-Orn-Pro-cha-Bta-Pcf-NH2 |
| 260 | Ac-Phe-Orn-Pro-cha-Bta-Pmf-NH2 |
| 261 | Ac-Phe-Orn-Pro-cha-Bta-Thi-NH2 |
| 262 | Ac-Phe-Orn-Pro-cha-Otf-Phe-NH2 |
| 263 | Ac-Phe-Orn-Pro-ctb-Bta-Phe-NH2 |
| 264 | Ac-Phe-Orn-Pro-ctb-Eaa-Phe-NH2 |
| 265 | Ac-Phe-Orn-Pro-ctb-Mcf-Phe-NH2 |
| 266 | Ac-Phe-Orn-Pro-ctb-Pff-Phe-NH2 |
| 267 | Ac-Phe-Orn-Pro-hch-Trp-Phe-OH |
| 268 | Ac-Phe-Orn-Pro-hle-1Ni-Phe-NH2 |
| 269 | Ac-Phe-Orn-Pro-hle-6FW-Phe-NH2 |
| 270 | Ac-Phe-Orn-Pro-hle-Bta-1Ni—NH2 |
| 271 | Ac-Phe-Orn-Pro-hle-Bta-2Ni—NH2 |
| 272 | Ac-Phe-Orn-Pro-hle-Bta-5Ff-NH2 |
| 273 | Ac-Phe-Orn-Pro-hle-Bta-Aic-NH2 |
| 274 | Ac-Phe-Orn-Pro-hle-Bta-Cha-NH2 |
| 275 | Ac-Phe-Orn-Pro-hle-Bta-Chg-NH2 |
| 276 | Ac-Phe-Orn-Pro-hle-Bta-Eaa-NH2 |
| 277 | Ac-Phe-Orn-Pro-hle-Bta-Egy-NH2 |
| 278 | Ac-Phe-Orn-Pro-hle-Bta-Pcf-NH2 |
| 279 | Ac-Phe-Orn-Pro-hle-Bta-Pff-NH2 |
| 280 | Ac-Phe-Orn-Pro-hle-Bta-Phe-NH2 |
| 281 | Ac-Phe-Orn-Pro-hle-Bta-phe-OH |
| 282 | Ac-Phe-Orn-Pro-hle-Bta-Tyr-NH2 |
| 283 | Ac-Phe-Orn-Pro-hle-Dff-Phe-NH2 |
| 284 | Ac-Phe-Orn-Pro-hle-Eaa-Phe-NH2 |
| 285 | Ac-Phe-Orn-Pro-hle-Egc-Phe-NH2 |
| 286 | Ac-Phe-Orn-Pro-hle-Egy-Phe-NH2 |
| 287 | Ac-Phe-Orn-Pro-hle-Egz-Phe-NH2 |
| 288 | Ac-Phe-Orn-Pro-hle-Mcf-2Ni—NH2 |
| 289 | Ac-Phe-Orn-Pro-hle-Mcf-Cha-NH2 |
| 290 | Ac-Phe-Orn-Pro-hle-Mcf-Pff-NH2 |
| 291 | Ac-Phe-Orn-Pro-hle-Mcf-Phe-NH2 |
| 292 | Ac-Phe-Orn-Pro-hle-Mff-Phe-NH2 |
| 293 | Ac-Phe-Orn-Pro-hle-Mmy-Phe-NH2 |
| 294 | Ac-Phe-Orn-Pro-hle-Ocf-Phe-NH2 |
| 295 | Ac-Phe-Orn-Pro-hle-Off-Phe-NH2 |
| 296 | Ac-Phe-Orn-Pro-hle-Otf-Phe-NH2 |
| 297 | Ac-Phe-Orn-Pro-hle-Pff-2Ni—NH2 |
| 298 | Ac-Phe-Orn-Pro-hle-Pff-Cha-NH2 |
| 299 | Ac-Phe-Orn-Pro-hle-Pff-Eaa-NH2 |
| 300 | Ac-Phe-Orn-Pro-hle-Pff-Mmy-NH2 |
| 301 | Ac-Phe-Orn-Pro-hle-Pff-Pff-NH2 |
| 302 | Ac-Phe-Orn-Pro-hle-Pff-Phe-NH2 |
| 304 | Ac-Phe-Orn-Pro-hle-Phe-Phe-NH2 |
| 305 | Ac-Phe-Orn-Pro-hle-Tff-Phe-NH2 |
| 306 | Ac-Phe-Orn-Pro-hle-Trp-Phe-NH2 |
| 307 | Ac-Phe-Orn-Pro-ile-Trp-Phe-NH2 |
| 308 | Ac-Phe-Orn-Pro-omf-Bta-Phe-NH2 |
| 309 | Ac-Phe-Orn-Ser-cha-Bta-Phe-NH2 |
| 310 | Ac-Ser-Phe-Orn-Aze-cha-Bta-Phe-NH2 |
| 311 | Ac-Thi-[Orn-Pro-hle-Bta-Phe] |
| 312 | Ac-Thi-Orn-Pro-cha-Bta-Phe-NH2 |
| 313 | Ac-Thi-Orn-Pro-cha-Bta-Thi-NH2 |
| 314 | Ac-Thr-Phe-Orn-Aze-cha-Bta-Phe-NH2 |
| 315 | Bzl-[Orn-Pro-cha-Bta-Nle] |
| 316 | CH3CH2CO-Phe-Orn-Pro-cha-Bta-Phe-NH2 |
| 317 | Def-[Orn-Ser-hle-Trp-Nle] |
| 318 | Eby-Phe-[Orn-Hyp-cha-Trp-Phe] |
| 319 | Eth-Phe-[Orn-Pro-hle-Pff-Nle] |
| 320 | FAc-Phe-Fib-Aze-cha-Bta-Phe-NH2 |
| 321 | FAc-Phe-Orn-Aze-cha-Bta-Phe-NH2 |
| 322 | FAc-Phe-Orn-Pro-cha-Bta-Phe-NH2 |
| 323 | Fai-Phe-[Orn-Hyp-cha-Trp-Phe] |
| 324 | Faz-Orn-Pro-cha-Bta-Phe-NH2 |
| 325 | Fbi-Phe-[Orn-Pro-cha-Trp-Nle] |
| 326 | Fbn-Phe-[Orn-Hyp-cha-Trp-Phe] |
| 327 | Fbn-Phe-[Orn-Pro-cha-Trp-Nle] |
| 328 | Fbn-Phe-[Orn-Pro-cha-Trp-Nle] |
| 329 | Fbn-Phe-Cit-Pro-hle-Bta-Phe-NH2 |
| 330 | Fbo-Phe-[Orn-Pro-cha-Trp-Nle] |
| 331 | Fbp-[Orn-Pro-cha-Trp-Nle] |
| 332 | Fci-[Phe-Orn-Hyp-cha-Trp-Phe] |
| 333 | Fck-[Phe-Orn-Pro-cha-Trp-Nle] |
| 334 | Fck-Phe-[Orn-Pro-cha-Trp-Nle] |
| 335 | Fha-Phe-[Orn-Hyp-cha-Trp-Phe] |
| 336 | Fhb-[Phe-Orn-Hyp-cha-Trp-Phe] |
| 337 | Fhi-Phe-[Orn-Hyp-cha-Trp-Phe] |
| 338 | Fhu-Phe-[Orn-Pro-hle-Pff-Nle] |
| 339 | Fhu-Phe-Orn-Pro-cha-Bta-Phe-NH2 |
| 340 | Fid-Phe-Orn-Pro-cha-Bta-Phe-NH2 |
| 341 | H-Amf-[Orn-Aze-hle-Pff-Nle] |
| 342 | H-Bal-Phe-[Orn-Hyp-hle-Trp-Nle] |
| 343 | H-Bal-Phe-[Orn-Pro-hle-Pff-Nle] |
| 344 | H-Eby-[Orn-Hyp-hle-Trp-Nle] |
| 345 | H-Gly-Phe-Orn-Pro-cha-Bta-Phe-NH2 |
| 346 | H-Nip-Phe-Cit-Pro-hle-Bta-Phe-NH2 |
| 347 | Hoo-Phe-[Orn-Hyp-hle-Pff-Nle] |
| 348 | Hoo-Phe-Cit-Pro-hle-Pff-Phe-NH2 |
| 349 | Hoo-Phe-Orn-Hyp-hle-Pff-Phe-NH2 |
| 350 | Hoo-Phe-Orn-Pro-hle-Bta-Phe-NH2 |
| 351 | Hoo-Phe-Orn-Pro-hle-Mcf-Phe-NH2 |
| 352 | Hoo-Phe-Orn-Pro-hle-Pff-Phe-NH2 |
| 353 | H-Phe-[Lys-Hyp-hle-Pff-Nle] |
| 354 | H-Phe-[Orn-Hym-hle-Mcf-Nle] |
| 355 | H-Phe-[Orn-Hym-hle-Pff-Phe] |
| 356 | H-Phe-[Orn-Hyp-cha-Trp-Nle] |
| 357 | H-Phe-[Orn-Hyp-cha-Trp-Phe] |
| 358 | H-Phe-[Orn-Hyp-ctb-Pff-Nle] |
| 359 | H-Phe-[Orn-Hyp-ctb-Trp-Nle] |
| 360 | H-Phe-[Orn-Hyp-ctb-Trp-Phe] |
| 361 | H-Phe-[Orn-Hyp-hle-Mcf-Leu] |
| 362 | H-Phe-[Orn-Hyp-hle-Pff-Chg] |
| 363 | H-Phe-[Orn-Hyp-hle-Pff-Hle] |
| 364 | H-Phe-[Orn-Hyp-hle-Pff-Leu] |
| 365 | H-Phe-[Orn-Hyp-hle-Pff-Nle] |
| 366 | H-Phe-[Orn-Hyp-hle-Pff-Phe] |
| 367 | H-Phe-[Orn-Hyp-hle-Trp-Hle] |
| 368 | H-Phe-[Orn-Hyp-hle-Trp-Leu] |
| 369 | H-Phe-[Orn-Hyp-hle-Trp-Nle] |
| 370 | H-Phe-[Orn-Hyp-hle-Trp-Nva] |
| 371 | H-Phe-[Orn-Hyp-hle-Trp-Phe] |
| 372 | H-Phe-[Orn-NMS-cha-Trp-Nle] |
| 373 | H-Phe-[Orn-NMS-hle-Pff-Phe] |
| 374 | H-Phe-[Orn-Pro-cha-Pff-Nle] |
| 375 | H-Phe-[Orn-Pro-cha-Pff-Phe] |
| 376 | H-Phe-[Orn-Pro-cha-Trp-Nle] |
| 377 | H-Phe-[Orn-Pro-hle-Mcf-Phe] |
| 378 | H-Phe-[Orn-Pro-hle-Ocf-Phe] |
| 379 | H-Phe-[Orn-Pro-hle-Pff-Nle] |
| 380 | H-Phe-[Orn-Pro-hle-Pff-Phe] |
| 381 | H-Phe-[Orn-Pro-hle-Trp-Nle] |
| 382 | H-Phe-[Orn-Ser-cha-Trp-Nle] |
| 383 | H-Phe-[Orn-Ser-cha-Trp-Phe] |
| 384 | H-Phe-[Orn-Ser-hle-Eaa-Nle] |
| 385 | H-Phe-[Orn-Ser-hle-Mcf-Leu] |
| 386 | H-Phe-[Orn-Ser-hle-Ocf-Nle] |
| 387 | H-Phe-[Orn-Ser-hle-Pff-Leu] |
| 388 | H-Phe-[Orn-Ser-hle-Pff-Nle] |
| 389 | H-Phe-[Orn-Ser-hle-Pff-Phe] |
| 390 | H-Phe-[Orn-Ser-hle-Trp-Nle] |
| 391 | H-Phe-Cit-Pro-hle-Bta-Phe-NH2 |
| 392 | Ohf-[Orn-Hyp-hle-Trp-Nle] |
| 393 | Tmg-Phe-[Orn-Hyp-cha-Trp-Phe] |

In an eleventh aspect of the invention the problem is solved by a pharmaceutical composition comprising at least one compound according to any of the aspects of the present invention and a pharmaceutically acceptable carrier.

In a twelfth aspect of the invention the problem is solved by the use of at least one of the compounds of any of the first to the tenth aspect of the present invention for the manufacture of a medicament.

In an embodiment of the twelfth aspect the medicament is used or useful for the prevention and/or treatment of a condition associated with complement activation and/or a condition where the inhibition of the complement system leads to a relief of the symptoms.

In an embodiment of the twelfth aspect the medicament is used or useful for the prevention and/or treatment of a condition where the inhibition of the C5a receptor alone or in combination with other therapeutics leads to a relief of the symptoms.

In an embodiment of the twelfth aspect the condition and/or the symptoms to be treated are selected from the group comprising autoimmune diseases, acute inflammatory diseases, trauma, local inflammations, shock and burn.

In an embodiment of the twelfth aspect the condition is selected from the group comprising sarcoidosis, septic shock, haemorrhagic shock, systemic inflammatory response syndrome (SIRS), multiple organ failure (MOF), asthma, vasculitis, myocarditis, dermatomyositis, inflammatory bowel disease (IBD), pemphigus, glomerulonephritis, acute respiratory insufficiency, stroke, myocardial infarction, reperfusion injury, neurocognitive dysfunction, burn, inflammatory diseases of the eye, local manifestations of systemic diseases, inflammatory diseases of the vessels, and acute injuries of the central nervous system.

In a preferred embodiment of the twelfth aspect the inflammatory disease of the eye is selected from the group comprising uveitis, age-related macular degeneration, diabetic retinopathy, diabetic macular edema, ocular pemphigoid, keratoconjunctivitis, Stevens-Johnson syndrome, and Graves ophthalmopathy.

In an alternative preferred embodiment of the twelfth aspect the condition is a local manifestation of a systemic disease, whereby the systemic disease is selected from the group comprising rheumatoid arthritis, SLE, type I diabetes, and type II diabetes.

In a more preferred embodiment of the twelfth aspect the manifestations are selected from the group comprising manifestations at the eye, at or in the brain, at the vessels, at the heart, at the lung, at the kidneys, at the liver, at the gastrointestinal tract, at the spleen, at the skin, at the skeletal system, in the lymphatic system, and in the blood.

In an embodiment of the twelfth aspect the autoimmune disease is selected from the group comprising alopecia greata, cold agglutinin immunohemolytic anemia, warm antibody immunohemolytic anemia, pernicious anemia (Biermer's Disease, Addison's anemia), antiphospholipid antibody syndrome (APS), arteriitis temporalis, atherosclerosis, autoimmune adrenalitis (Addison's disease), chronic fatigue syndrome (CFIDS), chronic-inflammatory polyneuropathy, Churg-Strauss syndrome, Cogan's syndrome, ulcerative colitis, CREST syndrome, diabetes mellitus type I, dermatitis herpetiformis, dermatomyositis, fibromyalgia, chronic autoimmune gastritis, Goodpasture syndrome (anti-GBM antibody related glomerulonephritis), Guillain-Barré syndrome (GBS; Polyradiculoneuropathy), Hashimoto thyroiditis, autoimmune hepatitis, idiopathic pulmonary fibrosis, autoimmune thrombocytopenice purpura (Werlhof's Disease), autoimmune infertility, autoimmune inner ear deafness (AIED), juvenile rheumatoid arthritis, autoimmune cardiomyopathy, Lambert-Eaton syndrome, Lichen sclerosus, Lupus erythematosus, Lyme arthritis, collagenosis, Graves' disease, Behcet disease, Crohn's disease, rheumatoid spondylitis, Méniére's disease, Reiter's disease, multiple sclerosis (MS, encephalomyelitis), myasthenia gravis, sympathetic ophtalmy, scaring pemphigoid, bullous pemphigoid, pemphigus vulgaris, polyarteritis nodosa, polychondritis, polyglandular autoimmune (PGA) syndrome, polymyalgia rheumatica, polymyositis, primary biliary cirrhosis, psoriasis, rheumatic fever, rheumatoid arthritis, sarcoidosis (Besnier-Boeck-Schaumann's disease), Sjörgen syndrome, scleroderma, sprue, stiff-man syndrome, systemic lupus erythematosus, Takayasu arteritis, transiente gluten intolerance, autoimmune uveitis, vasculites and vitiligo

In a preferred embodiment of the twelfth aspect the inflammatory diseases of the vessels are selected from the group comprising vasculitis, vascular leakage, and atherosclerosis.

In a more preferred embodiment of the twelfth aspect the vasculitis is selected from the group comprising primary and secondary vasculites.

In an embodiment of the twelfth aspect the primary vasculitis is selected from the group comprising Wegener's disease, Churg Strauss syndrome and microscopic polyangitis.

In an embodiment of the twelfth aspect the secondary vasculitis is selected from the group comprising vasculites induced through drug use and vasculitis induced by other diseases.

In an embodiment of the twelfth aspect the disease is selected from the group comprising AIDS, hepatitis B, hepatitis C and cytomegalovirus infection.

In an embodiment of the twelfth aspect the medicament is used to influence the adaptive and the innate immune system or useful for such a purpose.

In an embodiment of the twelfth aspect the influence is a strengthening, preferably of the immune system.

In an embodiment of the twelfth aspect the medicament is for the prevention and/or support of surgical manipulations.

In an embodiment of the twelfth aspect the medicament is used or useful for the prevention, support and/or post-operative treatment of a surgical manipulation, whereby the surgical manipulation is selected from the group comprising CABG, PACT, PTA, MidCAB, OPCAB, thrombolysis, organ transplantation, and vessel clamping.

In an embodiment of the twelfth aspect the medicament is used or useful for thrombolytic treatment.

In an embodiment of the twelfth aspect the medicament is used or useful in the settings of dialysis before, during, and/or after the treatment, preferably dialysis treatment.

In an embodiment the medicament is used or useful for the prevention of organ damage of a transplanted organ or of an organ that will be transplanted.

In an embodiment the medicament is used or useful for the prevention or treatment of transplant rejection.

In a still further aspect the present invention is related to a method for the treatment of patients, whereby the method comprises the administration of one or several of the compounds according to the present invention. The treatment can be a treatment in the narrower sense, however, may also include a preventive treatment. In an embodiment of the method the method is the treatment of a CPB (cardiopulmonary bypass) patients, which are to be protected against neurocognitive dysfunction by a preventive administration of the inhibitors according to the invention.

The patient to be treated is preferably a mammal, more preferably a domestic farming animal, sports animal and pet, and most preferably a human being. In a preferred embodiment the patient is a patient in need of such treatment. In a further preferred embodiment the patient is suffering from one of the above mentioned diseases for the treatment of which the compounds according to the present invention may be used.

The present invention provides for the first time such antagonists for the C5a receptor that overcome the inherent pharmacological disadvantages of the antagonistic peptides of the prior art which contain a positive charge.

The invention is based on the surprising finding that in contrast to the technical teaching of the prior art, also antagonists for the C5a receptor can be obtained which, under physiological conditions, especially at a pH of 7.4, do not have a positive net charge and/or whose C-terminal amino acid does not possess a positive charge under physiological conditions.

According to the understanding of the inventors the positive charge in peptides can be very disadvantageous from a pharmacological point of view. Positive charges can, e.g., lead to histamine release and cause lower membrane permeability (see example 15). Therefore, it is particularly desired to develop a peptidic antagonist that does not possess a positive net charge (in the following also referred to as compound).

Additionally, the avoidance of a C-terminal positive charge can have further positive effects: For example, receptor specificity or important in vivo parameters like pharmacokinetics, plasma protein binding or mutagenicity can be positively influenced.

The compounds which are disclosed in the present invention were tested in a primary assay for their IC₅₀ values in a functional assay system. Preferably, all compounds, peptides and peptidomimetics are regarded to have noteworthy inhibitory activity in the sense of the present invention, that have an IC₅₀ value of less than 200 nM in a functional assay system as described in example 1.

In particular the compounds of the invention are C5a receptor antagonists. Even more preferably they are peptides or peptidomimetics. Furthermore, the invention is based on the surprising finding that the compounds which are used in accordance with the present invention as C5a receptors posses an uncharged C-terminal amino acid, amino acid derivative or amino acid analog.

Particularly preferred compounds and antagonists according to the present invention are the following cyclic compounds.

| Nr. | Compound |
|---|---|
| 1 | Ac-Phe-[Orn-Pro-cha-Trp-Phe] |
| 2 | Ac-Phe-[Orn-Hyp-cha-Trp-Phe] |
| 3 | HOCH2(CHOH)4—C═N—O—CH2—CO-Phe-[Orn-Pro-cha-Trp- |
| | Nle] |
| 4 | X-Phe-[Orn-Pro-cha-Trp-Nle]; X = 2-Acetamido-1-Methyl- |
| | Glucuronyl |
| 5 | Ac-Phe-[Orn-Hyp(COCH2OCH2CH2OCH2CH2OCH3)- |
| | cha-Trp-Nle] |
| 6 | Ac-Phe-[Orn-Hyp(CONH—CH2CH(OH)—CH2OH)-cha-Trp- |
| | Nle] |
| 20 | Ac-Phe-[Orn-Pro-cha-Trp-Ecr] |
| 28 | Ac-Phe-[Orn-Pro-cha-Trp-Nle] |
| 29 | Ac-Phe-[Orn-Pro-cha-Trp-Met] |
| 31 | Ac-Phe-[Orn-Pro-cha-Trp-Nva] |
| 32 | Ac-Phe-[Orn-Pro-cha-Trp-Hle] |
| 33 | Ac-Phe-[Orn-Pro-cha-Trp-Eaf] |
| 34 | Ac-Phe-[Orn-Pro-cha-Trp-Ebd] |
| 35 | Ac-Phe-[Orn-Pro-cha-Trp-Eag] |
| 36 | Ac-Phe-[Orn-Pro-cha-Trp-pmf] |
| 37 | Ac-Phe-[Orn-Pro-cha-Trp-2Ni] |
| 38 | Ac-Phe-[Orn-Pro-cha-Trp-Thi] |
| 41 | Ph-CH2—CH2—CO-[Orn-Pro-cha-Trp-Nle] |
| 42 | H-Phe-[Orn-Pro-cha-Trp-Nle] |
| 43 | Ac-Lys-Phe-[Orn-Pro-cha-Trp-Nle] |
| 44 | H-Phe-[Orn-Ser-cha-Trp-Nle] |
| 56 | Ph-CH2-[Orn-Pro-cha-Trp-Nle] |
| 57 | Ph-CH2-[Orn-Pro-cha-Trp-Phe] |
| 58 | Ac-Phe-[Orn-Pro-cha-Trp-1Ni] |
| 59 | Ph-CH(OH)—CH2—CO-[Orn-Pro-cha-Trp-Nle] |
| 144 | Ac-Phe-[Orn-Hyp-cha-Trp-Nle] |
| 145 | 3PP-[Orn-Hyp-cha-Trp-Nle] |
| 146 | Ac-Phe-[Orn-Pro-cha-Trp-Tyr] |
| 147 | Ac-Phe-[Orn-Pro-omf-Trp-Nle] |
| 172 | Ac-Phe-[Cys-Pro-cha-Bta-Phe-Cys]-NH2 |
| 173 | Ac-Phe-[Orn-Asn-cha-Trp-Nle] |
| 174 | Ac-Phe-[Orn-Aze-cha-Trp-Nle] |
| 175 | Ac-Phe-[Orn-Chy-cha-Trp-Nle] |
| 176 | Ac-Phe-[Orn-HyA-cha-Trp-Phe] |
| 177 | Ac-Phe-[Orn-Hyp-hle-Bta-Phe] |
| 178 | Ac-Phe-[Orn-Hyp-hle-Mcf-Phe] |
| 179 | Ac-Phe-[Orn-Hyp-hle-Pff-Nle] |
| 180 | Ac-Phe-[Orn-Hyp-hle-Pff-Phe] |
| 181 | Ac-Phe-[Orn-Hyp-hle-Trp-Phe] |
| 182 | Ac-Phe-[Orn-Hyp-Mmf-Trp-Nle] |
| 183 | Ac-Phe-[Orn-Hyp-Mmf-Trp-Phe] |
| 184 | Ac-Phe-[Orn-NMD-cha-Trp-Nle] |
| 185 | Ac-Phe-[Orn-Pip-hle-Bta-Phe] |
| 186 | Ac-Phe-[Orn-Pro-cha-Pff-Nle] |
| 187 | Ac-Phe-[Orn-Pro-cha-Pff-Phe] |
| 188 | Ac-Phe-[Orn-Pro-cha-Trp-1Ni] |
| 189 | Ac-Phe-[Orn-Pro-cha-Trp-Cha] |
| 190 | Ac-Phe-[Orn-Pro-cha-Trp-Chg] |
| 192 | Ac-Phe-[Orn-Pro-cha-Trp-Ecr] |
| 193 | Ac-Phe-[Orn-Pro-cha-Trp-Leu] |
| 194 | Ac-Phe-[Orn-Pro-cha-Trp-nle] |
| 195 | Ac-Phe-[Orn-Pro-cha-Trp-Phe] |
| 196 | Ac-Phe-[Orn-Pro-hle-Bta-Nle] |
| 197 | Ac-Phe-[Orn-Pro-hle-Bta-Phe] |
| 198 | Ac-Phe-[Orn-Pro-hle-Pff-Phe] |
| 199 | Ac-Phe-[Orn-Pro-hle-Trp-Nle] |
| 200 | Ac-Phe-[Orn-Ser-cha-Trp-Nle] |
| 201 | Ac-Phe-[Orn-Ser-cha-Trp-Nle] |
| 202 | Ac-Phe-[Orn-Ser-hle-Trp-Nle] |
| 203 | Ac-Phe-[Orn-Thr-cha-Trp-Nle] |
| 204 | Ac-Phe-[Orn-Tic-cha-Trp-Nle] |
| 205 | Ac-Phe-[Orn-Tic-cha-Trp-Nle] |
| 311 | Ac-Thi-[Orn-Pro-hle-Bta-Phe] |
| 315 | Bzl-[Orn-Pro-cha-Bta-Nle] |
| 317 | Def-[Orn-Ser-hle-Trp-Nle] |
| 318 | Eby-Phe-[Orn-Hyp-cha-Trp-Phe] |
| 319 | Eth-Phe-[Orn-Pro-hle-Pff-Nle] |
| 323 | Fai-Phe-[Orn-Hyp-cha-Trp-Phe] |
| 325 | Fbi-Phe-[Orn-Pro-cha-Trp-Nle] |
| 326 | Fbn-Phe-[Orn-Hyp-cha-Trp-Phe] |
| 327 | Fbn-Phe-[Orn-Pro-cha-Trp-Nle] |
| 328 | Fbn-Phe-[Orn-Pro-cha-Trp-Nle] |
| 330 | Fbo-Phe-[Orn-Pro-cha-Trp-Nle] |
| 331 | Fbp-[Orn-Pro-cha-Trp-Nle] |
| 332 | Fci-[Phe-Orn-Hyp-cha-Trp-Phe] |
| 333 | Fck-[Phe-Orn-Pro-cha-Trp-Nle] |
| 334 | Fck-Phe-[Orn-Pro-cha-Trp-Nle] |
| 335 | Fha-Phe-[Orn-Hyp-cha-Trp-Phe] |
| 336 | Fhb-[Phe-Orn-Hyp-cha-Trp-Phe] |
| 337 | Fhi-Phe-[Orn-Hyp-cha-Trp-Phe] |
| 338 | Fhu-Phe-[Orn-Pro-hle-Pff-Nle] |
| 341 | H-Amf-[Orn-Aze-hle-Pff-Nle] |
| 342 | H-Bal-Phe-[Orn-Hyp-hle-Trp-Nle] |
| 343 | H-Bal-Phe-[Orn-Pro-hle-Pff-Nle] |
| 344 | H-Eby-[Orn-Hyp-hle-Trp-Nle] |
| 347 | Hoo-Phe-[Orn-Hyp-hle-Pff-Nle] |
| 353 | H-Phe-[Lys-Hyp-hle-Pff-Nle] |
| 354 | H-Phe-[Orn-Hym-hle-Mcf-Nle] |
| 355 | H-Phe-[Orn-Hym-hle-Pff-Phe] |
| 356 | H-Phe-[Orn-Hyp-cha-Trp-Nle] |
| 357 | H-Phe-[Orn-Hyp-cha-Trp-Phe] |
| 358 | H-Phe-[Orn-Hyp-ctb-Pff-Nle] |
| 359 | H-Phe-[Orn-Hyp-ctb-Trp-Nle] |
| 360 | H-Phe-[Orn-Hyp-ctb-Trp-Phe] |
| 361 | H-Phe-[Orn-Hyp-hle-Mcf-Leu] |
| 362 | H-Phe-[Orn-Hyp-hle-Pff-Chg] |
| 363 | H-Phe-[Orn-Hyp-hle-Pff-Hle] |
| 364 | H-Phe-[Orn-Hyp-hle-Pff-Leu] |
| 365 | H-Phe-[Orn-Hyp-hle-Pff-Nle] |
| 366 | H-Phe-[Orn-Hyp-hle-Pff-Phe] |
| 367 | H-Phe-[Orn-Hyp-hle-Trp-Hle] |
| 368 | H-Phe-[Orn-Hyp-hle-Trp-Leu] |
| 369 | H-Phe-[Orn-Hyp-hle-Trp-Nle] |
| 370 | H-Phe-[Orn-Hyp-hle-Trp-Nva] |
| 371 | H-Phe-[Orn-Hyp-hle-Trp-Phe] |
| 372 | H-Phe-[Orn-NMS-cha-Trp-Nle] |
| 373 | H-Phe-[Orn-NMS-hle-Pff-Phe] |
| 374 | H-Phe-[Orn-Pro-cha-Pff-Nle] |
| 375 | H-Phe-[Orn-Pro-cha-Pff-Phe] |
| 376 | H-Phe-[Orn-Pro-cha-Trp-Nle] |
| 377 | H-Phe-[Orn-Pro-hle-Mcf-Phe] |
| 378 | H-Phe-[Orn-Pro-hle-Ocf-Phe] |
| 379 | H-Phe-[Orn-Pro-hle-Pff-Nle] |
| 380 | H-Phe-[Orn-Pro-hle-Pff-Phe] |
| 381 | H-Phe-[Orn-Pro-hle-Trp-Nle] |
| 382 | H-Phe-[Orn-Ser-cha-Trp-Nle] |
| 383 | H-Phe-[Orn-Ser-cha-Trp-Phe] |
| 384 | H-Phe-[Orn-Ser-hle-Eaa-Nle] |
| 385 | H-Phe-[Orn-Ser-hle-Mcf-Leu] |
| 386 | H-Phe-[Orn-Ser-hle-Ocf-Nle] |
| 387 | H-Phe-[Orn-Ser-hle-Pff-Leu] |
| 388 | H-Phe-[Orn-Ser-hle-Pff-Nle] |
| 389 | H-Phe-[Orn-Ser-hle-Pff-Phe] |
| 390 | H-Phe-[Orn-Ser-hle-Trp-Nle] |
| 392 | Ohf-[Orn-Hyp-hle-Trp-Nle] |
| 393 | Tmg-Phe-[Orn-Hyp-cha-Trp-Phe] |

In connection with the present invention, however, it was also surprisingly found that linear, thus structurally flexible peptides can be as potent inhibitors as structurally fixed cyclic peptides. The reason for this may be the substitution of the C-terminal charged arginine by hydrophobic amino acids, amino acid derivatives or amino acid analogs. Examples for such linear peptidic inhibitors according to the invention are in particular the compounds shown in the following table:

| | |
|---|---|
| 51 | Ac-Phe-Orn-Pro-cha-Trp-Phe-NH2 |
| 52 | Ac-Phe-Orn-Aze-cha-Bta-Phe-NH2 |
| 53 | Ac-Phe-Orn-Pro-cha-Bta-2Ni—NH2 |
| 54 | Ac-Phe-Orn-Pro-cha-Bta-Cha-NH2 |
| 55 | Ac-Phe-Orn-Pip-cha-Trp-Phe-NH2 |
| 61 | Ac-Phe-Orn-Pro-cha-Trp-Phe-NH2 |
| 62 | Ac-Phe-Orn-Pro-cha-Bta-Phe-NH2 |
| 64 | Ac-Phe-Orn-Pro-cha-Trp-2Ni—NH2 |
| 65 | Ac-Phe-Orn-Pro-cha-Trp-Cha-NH2 |
| 66 | Ac-Thi-Orn-Aze-cha-Bta-Phe-NH2 |
| 67 | Ac-Thi-Orn-Pip-cha-Bta-Phe-NH2 |
| 68 | Ac-Phe-Orn-Pro-cha-Trp-Eap-NH2 |
| 69 | Me2-Phe-Orn-Pro-cha-Trp-Phe-NH2 |
| 70 | Ph2-CH—CH2—CO-Orn-Pro-cha-Trp-Phe-NH2 |
| 71 | Ac-Ebw-Orn-Pro-cha-Trp-Phe-NH2 |
| 72 | Ac-Phe-Orn-Pro-cha-Trp-NH—CH2—CH2-Ph |
| 73 | Ac-Phe-Orn-Aze-cha-Bta-NH—CH2—CH2-Ph |
| 74 | H-Phe-Orn-Pro-cha-Trp-Phe-NH2 |
| 75 | H-Me-Phe-Orn-Pro-cha-Trp-Phe-NH2 |
| 76 | Bu-NH—CO-Phe-Orn-Pro-cha-Trp-Phe-NH2 |
| 77 | Ac-Thi-Orn-Pro-cha-Trp-Phe-NH2 |
| 78 | Ac-Ebw-Orn-Pro-cha-Trp-Phe-NH2 |
| 79 | Ac-Phe-Orn-Ala-cha-Trp-Phe-NH2 |
| 80 | Ac-Phe-Orn-Pro-cha-Trp-Thi-NH2 |
| 81 | Ac-Phe-Orn-Aze-cha-Pcf-Phe-NH2 |
| 82 | Ac-Phe-Orn(Ac)-Pro-cha-Trp-Phe-NH2 |
| 83 | Ac-Phe-Orn-Aze-cha-Trp-Phe-NH2 |
| 84 | Ac-Phe-Trp-Pro-cha-Trp-Phe-NH2 |
| 85 | Ph-NH—CO-Phe-Orn-Pro-cha-Trp-Phe-NH2 |
| 86 | Bu-O—CO-Phe-Orn-Pro-cha-Trp-Phe-NH2 |
| 87 | Ac-Phe-Lys-Pro-cha-Trp-Phe-NH2 |
| 88 | Ac-Phe-Arg-Pro-cha-Trp-Phe-NH2 |
| 89 | Ac-Phe-Gln-Pro-cha-Trp-Phe-NH2 |
| 90 | Ac-Phe-Ser-Pro-cha-Trp-Phe-NH2 |
| 91 | Ac-Phe-Glu-Pro-cha-Trp-Phe-NH2 |
| 92 | Ac-Phe-Orn-Pip-cha-Trp-Phe-NH2 |
| 93 | Ac-Phe-Orn-Hyp-cha-Trp-Phe-NH2 |
| 94 | Ac-Phe-Orn-Pro-cha-Trp-1Ni—NH2 |
| 95 | Ac-Phe-Orn-Aze-cha-Bta-Phe-NH-Me |
| 96 | CH3—SO2-Phe-Orn-Aze-cha-Bta-Phe-NH2 |
| 99 | Ac-Phe-Orn-Aze-cha-Pff-Phe-NH2 |
| 100 | Ac-Phe-Orn-Aze-cha-Mcf-Phe-NH2 |
| 101 | Ac-Phe-Orn(Ac)-Aze-cha-Bta-Phe-NH2 |
| 102 | Ac-Ebw-Orn-Pro-cha-Trp-Phe-NH2 |
| 103 | Ac-Phe-Trp-Pro-cha-Trp-Phe-NH2 |
| 104 | Ac-Phe-Arg-Pro-cha-Trp-Phe-NH2 |
| 105 | Ac-Phe-Orn-Pip-cha-Trp-Phe-NH2 |
| 106 | 3PP-Orn-Aze-cha-Bta-Phe-NH2 |
| 107 | Ac-Phe-Orn-Tic-cha-Trp-Phe-NH2 |
| 108 | Ac-Phe-Orn-Ser-cha-Trp-Phe-NH2 |
| 109 | Ac-Phe-Orn-Pro-chg-Trp-Phe-NH2 |
| 110 | Ac-Phe-Orn-Pro-hch-Trp-Phe-NH2 |
| 111 | Ac-Phe-Orn-Pro-cha-Trp-Phg-NH2 |
| 112 | Ac-Phe-Bta-Aze-cha-Bta-Phe-NH2 |
| 113 | Ac-Phe-Trp-Pro-cha-Bta-Phe-NH2 |
| 115 | Ac-Phe-Orn-Pip-cha-Trp-Phe-OH |
| 116 | Ac-Phe-Orn-Tic-cha-Trp-Phe-OH |
| 117 | Ac-Phe-Orn-Ser-cha-Trp-Phe-OH |
| 118 | Ac-Phe-Orn-Pro-chg-Trp-Phe-OH |
| 119 | Ac-Phe-Eec-Pro-cha-Bta-Phe-NH2 |
| 120 | Ac-Phe-Nle-Pro-cha-Bta-Phe-NH2 |
| 121 | Ac-Phe-Har-Pro-cha-Bta-Phe-NH2 |
| 122 | Ac-Phe-Arg-Pro-cha-Bta-Phe-NH2 |
| 123 | Ac-Phe-Cys(Acm)-Pro-cha-Bta-Phe-NH2 |
| 124 | Ac-Phe-Mpa-Pro-cha-Bta-Phe-NH2 |
| 125 | Ac-Eby-Orn-Pro-cha-Bta-Phe-NH2 |
| 126 | Ac-Phg-Orn-Pro-cha-Bta-Phe-NH2 |
| 127 | Ac-Phe-Paf-Pro-cha-Bta-Phe-NH2 |
| 128 | H2N—CO-Phe-Orn-Pro-cha-Bta-Phe-NH2 |
| 129 | Me-O—CO-Phe-Orn-Pro-cha-Bta-Phe-NH2 |
| 130 | (—CO—CH2—NH—CO—)-Phe-Orn-Pro-cha-Bta-Phe-NH2 |
| 132 | Ac-Phe-Orn-Pro-hch-Trp-Phe-OH |
| 133 | (—CO—CH2—CH2—CO—)-Phe-Orn-Pro-cha-Bta-Phe-NH2 |
| 134 | tBu-CO-Phe-Orn-Pro-cha-Bta-Phe-NH2 |
| 135 | Ac-Lys-Phe-Orn-Aze-cha-Bta-Phe-NH2 |
| 136 | Ac-Gly-Phe-Orn-Aze-cha-Bta-Phe-NH2 |
| 137 | Ac-Arg-Phe-Orn-Aze-cha-Bta-Phe-NH2 |
| 138 | Ac-His-Phe-Orn-Aze-cha-Bta-Phe-NH2 |
| 139 | Ac-Ser-Phe-Orn-Aze-cha-Bta-Phe-NH2 |
| 140 | Ac-Guf-Phe-Orn-Aze-cha-Bta-Phe-NH2 |
| 141 | Ac-Dab-Phe-Orn-Aze-cha-Bta-Phe-NH2 |
| 142 | FH2C—CO-Phe-Orn-Pro-cha-Bta-Phe-NH2 |
| 143 | Ac-Phe-Orn(Et2)-Pro-cha-Trp-Phe-NH2 |
| 148 | Ac-Phe-N(nBu)-CH2—CO-Pro-cha-Trp-Phe-NH2 |
| 149 | Ac-Phe-Orn-Pro-hle-Bta-Phe-NH2 |
| 150 | Ac-Phe-Arg(CH2—CH2)-Pro-cha-Bta-Phe-NH2 |
| 151 | Ac-Ala-Phe-Orn-Aze-cha-Bta-Phe-NH2 |
| 152 | Ac-Arg-Phe-Orn-Aze-cha-Bta-Phe-NH2 |
| 153 | Ac-Cit-Phe-Orn-Aze-cha-Bta-Phe-NH2 |
| 154 | Ac-Gly-Phe-Orn-Aze-cha-Bta-Phe-NH2 |
| 155 | Ac-Gly-Phe-Orn-Aze-chg-Bta-Phe-NH2 |
| 156 | Ac-Gly-Phe-Orn-Aze-hch-Bta-Phe-NH2 |
| 157 | Ac-Gly-Thi-Orn-Aze-cha-Bta-Phe-NH2 |
| 158 | Ac-His-Phe-Orn-Aze-cha-Bta-Phe-NH2 |
| 159 | Ac-Hyp-Phe-Orn-Aze-cha-Bta-Phe-NH2 |
| 160 | Ac-Lys-Phe-Orn-Aze-cha-Bta-Phe-NH2 |
| 161 | Ac-Mff-Orn-Pro-cha-Bta-Phe-NH2 |
| 162 | Ac-Mff-Orn-Pro-hle-Bta-Phe-NH2 |
| 163 | Ac-Mff-Orn-Pro-hle-Mcf-Mff-NH2 |
| 164 | Ac-Mmy-Orn-Pro-hle-Pff-Phe-NH2 |
| 165 | Ac-NMF-Orn-Pro-cha-Bta-Phe-NH2 |
| 166 | Ac-Off-Orn-Pro-cha-Bta-Phe-NH2 |
| 167 | Ac-Off-Orn-Pro-hle-Bta-Phe-NH2 |
| 168 | Ac-Orn-Phe-Orn-Aze-cha-Bta-Phe-NH2 |
| 169 | Ac-Pff-Orn-Pro-cha-Bta-Phe-NH2 |
| 170 | Ac-Pff-Orn-Pro-hle-Bta-Phe-NH2 |
| 171 | Ac-Pff-Orn-Pro-hle-Mcf-Pff-NH2 |
| 206 | Ac-Phe-Ala-Pro-cha-Bta-Phe-NH2 |
| 207 | Ac-Phe-Arg-Pro-hle-Bta-Phe-NH2 |
| 208 | Ac-Phe-Arg-Pro-hle-Mcf-Phe-NH2 |
| 209 | Ac-Phe-Cit-Hyp-hle-Bta-Phe-NH2 |
| 210 | Ac-Phe-Cit-Pro-cha-Bta-Phe-NH2 |
| 211 | Ac-Phe-Cit-Pro-hle-Bta-Phe-NH2 |
| 212 | Ac-Phe-Cit-Ser-hle-Bta-Phe-NH2 |
| 213 | Ac-Phe-Dab-Aze-cha-Bta-Phe-NH2 |
| 214 | Ac-Phe-Dab-Aze-hle-Bta-Phe-NH2 |
| 215 | Ac-Phe-Dab-Pro-cha-Bta-Phe-NH2 |
| 216 | Ac-Phe-Dap-Pro-cha-Bta-Phe-NH2 |
| 217 | Ac-Phe-Ech-Pro-cha-Bta-Phe-NH2 |
| 218 | Ac-Phe-Eep-Pro-cha-Bta-Phe-NH2 |
| 219 | Ac-Phe-Fcn-Aze-cha-Bta-Phe-NH2 |
| 220 | Ac-Phe-Fcn-Pro-cha-Bta-Phe-NH2 |
| 221 | Ac-Phe-Fco-Pro-cha-Bta-Phe-NH2 |
| 222 | Ac-Phe-Fco-Pro-cha-Bta-Phe-NH2 |
| 223 | Ac-Phe-Fcp-Aze-cha-Bta-Phe-NH2 |
| 224 | Ac-Phe-Ffa-Aze-cha-Bta-Phe-NH2 |
| 225 | Ac-Phe-Ffa-Pro-cha-Bta-Phe-NH2 |
| 226 | Ac-Phe-Ffa-Pro-hle-Bta-Phe-NH2 |
| 227 | Ac-Phe-G23-Pro-cha-Bta-Phe-NH2 |
| 228 | Ac-Phe-Guf-Pro-cha-Bta-Phe-NH2 |
| 229 | Ac-Phe-Har-Aze-cha-Bta-Phe-NH2 |
| 230 | Ac-Phe-His-Pro-cha-Bta-Phe-NH2 |
| 231 | Ac-Phe-L22-Pro-cha-Bta-Phe-NH2 |
| 232 | Ac-Phe-OrA-Pro-cha-Bta-Phe-NH2 |
| 233 | Ac-Phe-OrE-Pro-cha-Bta-Phe-NH2 |
| 234 | Ac-Phe-Orn-Aze-hle-Bta-Phe-NH2 |
| 235 | Ac-Phe-Orn-Chy-cha-Bta-Phe-NH2 |
| 236 | Ac-Phe-Orn-Chy-hle-Pff-Phe-NH2 |
| 237 | Ac-Phe-Orn-G24-cha-Bta-Phe-NH2 |
| 238 | Ac-Phe-Orn-G25-cha-Bta-Phe-NH2 |
| 239 | Ac-Phe-Orn-G26-cha-Bta-Phe-NH2 |
| 240 | Ac-Phe-Orn-G27-cha-Bta-Phe-NH2 |
| 241 | Ac-Phe-Orn-G30-cha-Bta-Phe-NH2 |
| 242 | Ac-Phe-Orn-G31-cha-Bta-Phe-NH2 |
| 243 | Ac-Phe-Orn-Hse-cha-Bta-Phe-NH2 |
| 244 | Ac-Phe-Orn-Hyp-hle-Bta-Phe-NH2 |
| 245 | Ac-Phe-Orn-Hyp-hle-Pff-Phe-NH2 |
| 246 | Ac-Phe-Orn-NMA-cha-Bta-Phe-NH2 |
| 247 | Ac-Phe-Orn-NMS-cha-Bta-Phe-NH2 |
| 248 | Ac-Phe-Orn-Pro-cha-1Ni-Phe-NH2 |
| 249 | Ac-Phe-Orn-Pro-cha-Bta-1Ni—NH2 |
| 250 | Ac-Phe-Orn-Pro-cha-Bta-Bhf-NH2 |
| 251 | Ac-Phe-Orn-Pro-cha-Bta-Dff-NH2 |
| 252 | Ac-Phe-Orn-Pro-cha-Bta-Eaa-NH2 |
| 253 | Ac-Phe-Orn-Pro-cha-Bta-L19 |
| 254 | Ac-Phe-Orn-Pro-cha-Bta-Mcf-NH2 |
| 255 | Ac-Phe-Orn-Pro-cha-Bta-Mff-NH2 |
| 256 | Ac-Phe-Orn-Pro-cha-Bta-NH—CH(CH2OH)—CH2-Ph |
| 257 | Ac-Phe-Orn-Pro-Cha-Bta-NH-NBn-CO—NH2 |
| 258 | Ac-Phe-Orn-Pro-cha-Bta-Opa-NH2 |
| 259 | Ac-Phe-Orn-Pro-cha-Bta-Pcf-NH2 |
| 260 | Ac-Phe-Orn-Pro-cha-Bta-Pmf-NH2 |
| 261 | Ac-Phe-Orn-Pro-cha-Bta-Thi-NH2 |
| 262 | Ac-Phe-Orn-Pro-cha-Otf-Phe-NH2 |
| 263 | Ac-Phe-Orn-Pro-ctb-Bta-Phe-NH2 |
| 264 | Ac-Phe-Orn-Pro-ctb-Eaa-Phe-NH2 |
| 265 | Ac-Phe-Orn-Pro-ctb-Mcf-Phe-NH2 |
| 266 | Ac-Phe-Orn-Pro-ctb-Pff-Phe-NH2 |
| 267 | Ac-Phe-Orn-Pro-hch-Trp-Phe-OH |
| 268 | Ac-Phe-Orn-Pro-hle-1Ni-Phe-NH2 |
| 269 | Ac-Phe-Orn-Pro-hle-6FW-Phe-NH2 |
| 270 | Ac-Phe-Orn-Pro-hle-Bta-1Ni—NH2 |
| 271 | Ac-Phe-Orn-Pro-hle-Bta-2Ni—NH2 |
| 272 | Ac-Phe-Orn-Pro-hle-Bta-5Ff-NH2 |
| 273 | Ac-Phe-Orn-Pro-hle-Bta-Aic-NH2 |
| 274 | Ac-Phe-Orn-Pro-hle-Bta-Cha-NH2 |
| 275 | Ac-Phe-Orn-Pro-hle-Bta-Chg-NH2 |
| 276 | Ac-Phe-Orn-Pro-hle-Bta-Eaa-NH2 |
| 277 | Ac-Phe-Orn-Pro-hle-Bta-Egy-NH2 |
| 278 | Ac-Phe-Orn-Pro-hle-Bta-Pcf-NH2 |
| 279 | Ac-Phe-Orn-Pro-hle-Bta-Pff-NH2 |
| 280 | Ac-Phe-Orn-Pro-hle-Bta-Phe-NH2 |
| 281 | Ac-Phe-Orn-Pro-hle-Bta-phe-OH |
| 282 | Ac-Phe-Orn-Pro-hle-Bta-Tyr-NH2 |
| 283 | Ac-Phe-Orn-Pro-hle-Dff-Phe-NH2 |
| 284 | Ac-Phe-Orn-Pro-hle-Eaa-Phe-NH2 |
| 285 | Ac-Phe-Orn-Pro-hle-Egc-Phe-NH2 |
| 286 | Ac-Phe-Orn-Pro-hle-Egy-Phe-NH2 |
| 287 | Ac-Phe-Orn-Pro-hle-Egz-Phe-NH2 |
| 288 | Ac-Phe-Orn-Pro-hle-Mcf-2Ni—NH2 |
| 289 | Ac-Phe-Orn-Pro-hle-Mcf-Cha-NH2 |
| 290 | Ac-Phe-Orn-Pro-hle-Mcf-Pff-NH2 |
| 291 | Ac-Phe-Orn-Pro-hle-Mcf-Phe-NH2 |
| 292 | Ac-Phe-Orn-Pro-hle-Mff-Phe-NH2 |
| 293 | Ac-Phe-Orn-Pro-hle-Mmy-Phe-NH2 |
| 294 | Ac-Phe-Orn-Pro-hle-Ocf-Phe-NH2 |
| 295 | Ac-Phe-Orn-Pro-hle-Off-Phe-NH2 |
| 296 | Ac-Phe-Orn-Pro-hle-Otf-Phe-NH2 |
| 297 | Ac-Phe-Orn-Pro-hle-Pff-2Ni—NH2 |
| 298 | Ac-Phe-Orn-Pro-hle-Pff-Cha-NH2 |
| 299 | Ac-Phe-Orn-Pro-hle-Pff-Eaa-NH2 |
| 300 | Ac-Phe-Orn-Pro-hle-Pff-Mmy-NH2 |
| 301 | Ac-Phe-Orn-Pro-hle-Pff-Pff-NH2 |
| 302 | Ac-Phe-Orn-Pro-hle-Pff-Phe-NH2 |
| 304 | Ac-Phe-Orn-Pro-hle-Phe-Phe-NH2 |
| 305 | Ac-Phe-Orn-Pro-hle-Tff-Phe-NH2 |
| 306 | Ac-Phe-Orn-Pro-hle-Trp-Phe-NH2 |
| 307 | Ac-Phe-Orn-Pro-ile-Trp-Phe-NH2 |
| 308 | Ac-Phe-Orn-Pro-omf-Bta-Phe-NH2 |
| 309 | Ac-Phe-Orn-Ser-cha-Bta-Phe-NH2 |
| 310 | Ac-Ser-Phe-Orn-Aze-cha-Bta-Phe-NH2 |
| 312 | Ac-Thi-Orn-Pro-cha-Bta-Phe-NH2 |
| 313 | Ac-Thi-Orn-Pro-cha-Bta-Thi-NH2 |
| 314 | Ac-Thr-Phe-Orn-Aze-cha-Bta-Phe-NH2 |
| 316 | CH3CH2CO-Phe-Orn-Pro-cha-Bta-Phe-NH2 |
| 320 | FAc-Phe-Fib-Aze-cha-Bta-Phe-NH2 |
| 321 | FAc-Phe-Orn-Aze-cha-Bta-Phe-NH2 |
| 322 | FAc-Phe-Orn-Pro-cha-Bta-Phe-NH2 |
| 324 | Faz-Orn-Pro-cha-Bta-Phe-NH2 |
| 329 | Fbn-Phe-Cit-Pro-hle-Bta-Phe-NH2 |
| 339 | Fhu-Phe-Orn-Pro-cha-Bta-Phe-NH2 |
| 340 | Fid-Phe-Orn-Pro-cha-Bta-Phe-NH2 |
| 345 | H-Gly-Phe-Orn-Pro-cha-Bta-Phe-NH2 |
| 346 | H-Nip-Phe-Cit-Pro-hle-Bta-Phe-NH2 |
| 348 | Hoo-Phe-Cit-Pro-hle-Pff-Phe-NH2 |
| 349 | Hoo-Phe-Orn-Hyp-hle-Pff-Phe-NH2 |
| 350 | Hoo-Phe-Orn-Pro-hle-Bta-Phe-NH2 |
| 351 | Hoo-Phe-Orn-Pro-hle-Mcf-Phe-NH2 |
| 352 | Hoo-Phe-Orn-Pro-hle-Pff-Phe-NH2 |
| 391 | H-Phe-Cit-Pro-hle-Bta-Phe-NH2 |

The linear peptides known from the prior art such as Finch et al. 1999 Journal of Medicinical Chemistry 42: 1965-1974; Wong et al. 1999 IDrugs 2: 686-693, U.S. Pat. No. 4,692,511, U.S. Pat. No. 5,663,148, WO 90/09162, WO 92/11858, WO 92/12168, WO 92/21361, WO 94/07518, WO 94/07815, WO 95/25957, WO 96/06629, WO 99/00406, and WO 99/13899 are in general significantly worse antagonists of C5a compared to cyclic peptides which are described in WO 99/00406 (e.g. Ac-Phe-[Lys-Pro-cha-Trp-arg], Ac-Phe-[Orn-Pro-cha-Trp-arg], Ac-Phe-[Orn-Pro-cha-Trp-Arg], Ac-Phe-[Lys-Pro-cha-Trp-Arg]). The in terms of antagonistic activity most active linear peptide described in WO 99/00406 has the sequence Me-Phe-Lys-Pro-cha-Trp-arg and an IC₅₀ of 0.085 μM (measured with the cellular myeloperoxidase release assay with human PMNs). In contrast thereto, the comparable cyclic peptide Ac-Phe-[Lys-Pro-cha-Trp-arg] (also from WO 99/00406) has an IC₅₀ of 0.012 μM. In WO 99/00406 it is mentioned that the decreased structural flexibility of the cyclic peptide leads to the decrease, i.e. an improvement of the IC₅₀. This is reflected in the development of cyclic—meaning least flexible—inhibitors like Ac-Phe-[Lys-Pro-cha-Trp-arg] and Ac-Phe-[Orn-Pro-cha-Trp-Arg].

Thus, the inventors intentionally departed from the understanding of the prior art regarding at least one aspect of the present invention and accordingly provide a new class of compounds which can be used as C5aR antagonists.

The present invention describes for the first time peptidic and peptidomimetic C5aR antagonists having IC50s of <200 nM, which do not have a positive net charge under physiological pH values (pH 7.4) and/or which C-terminal amino acid does not carry a positive charge. The IC₅₀ value is determined using a functional assay (Köhl 1997 The Anaphylatoxins. In: Dodds, A. W., Sim, R. B. (Eds.), Complement: A Practical Approach. Oxford, pp. 135-163). The compounds according to the present invention can therefore be used as C5aR antagonists, especially under physiological conditions.

The compounds according to this invention do underline the finding that a suitable hydrophobic substitution of an aliphatic, aromatic or heteroaromatic kind can replace the C-terminal arginine of C5aR binding peptides.

Another feature of the compounds according to this invention, especially of the peptides and peptidomimetics, is the absence of agonistic activity in a cellular assay up to a concentration of at least 1430 nM. Example 12 shows by way of example results from measurements with selected peptides according to the present invention using a method for determinating C5aR agonistic activities. Obviously, the compounds according to the present invention do not show any agonistics activity up to the highest concentration used. Within the present invention the following compounds in accordance with the present invention are examples for peptides in accordance with the present invention which are pure antagonists: HOCH₂(CHOH)₄—C═N—O—CH₂—CO-Phe-[Orn-Pro-cha-Trp-Nle], Ph-CH₂—CH₂—CO—[Orn-Pro-cha-Trp-Nle], Ac-Phe-[Orn-Hyp-cha-Trp-Phe], H-Phe-[Orn-Pro-cha-Trp-Phe], Ac-Phe-[Orn-Pro-cha-Trp-Phe], Ac-Lys-Phe-[Orn-Pro-cha-Trp-Nle], H-Phe-[Orn-Pro-cha-Trp-Nle], H-Phe-[Orn-Ser-cha-Trp-Nle], Ac-Phe-[Orn-Pro-cha-Trp-Eaf], Ac-Phe-Orn-Pro-cha-Trp-Phe-NH₂, Ac-Phe-Orn-Pro-cha-Bta-Phe-NH₂, Ac-Ebw-Orn-Pro-cha-Trp-Phe-NH₂, Ac-Phe-Orn-cha-cha-Bta-Phe-NH₂, Ac-Phe-Arg-Pro-cha-Trp-Phe-NH₂, Ac-Phe-Orn-Pip-cha-Trp-Phe-NH₂, Ac-Phe-Orn-Aze-cha-Trp-Phe-NH₂, Ac-Phe-Trp-Pro-cha-Trp-Phe-NH₂, Ac-Thi-Orn-Pip-cha-Bta-Phe-NH₂ Ac-Phe-Orn-Pro-hle-Bta-Phe-NH₂, Ac-Phe-Arg(CH₂—CH₂)-Pro-cha-Bta-Phe-NH₂.

For a detailed analysis of the C5aR antagonism and the development of a pharmacophore model of the compound Ac-Phe-[Orn-Pro-cha-Trp-Arg] the amino acids Phe, Trp and Arg were replaced by L-alanine, Pro was replaced by NMe-alanine and cha was replaced by D-alanine (single substitutions). The resulting peptides were analysed with a functional assay with regard to their C5aR antagonistic activity (example 11). From this approach it is apparent that the substitution of the amino acid side chains of Trp, cha, and Phe by methyl groups results in a pronounced loss of activity (IC₅₀ values >30 μM). In contrast to that the activity of the antagonist Ac-Phe-[Orn-Pro-cha-Trp-Arg] is comparable to the activity of the molecule having Pro replaced by NMeAla (IC₅₀=20 nM compared to 25 nM). The substitution of Ala for Arg also leads to a significant loss in activity (IC₅₀=20 nM to IC₅₀=5.6 μM) which is nevertheless less pronounced than for the substitution of Trp and Phe.

Additional substitutions at the peptide Ac-Phe-[Orn-Pro-cha-Trp-Arg] and similar compounds lead to a number of peptides and peptidomimetics, respectively, which, surprisingly, have noteworthy activities (example 11). Especially the following peptides show noteworthy inhibitory activity: Ac-Phe-[Orn-Pro-cha-Trp-Phe], Ac-Phe-[Orn-Hyp-cha-Trp-Phe], Ac-Phe-[Orn-Pro-cha-Trp-Paf], Ac-Phe-[Orn-Pro-cha-Trp-Ecr], Ac-Phe-[Orn-Pro-cha-Trp-Ppa], Ac-Phe-[Orn-Pro-cha-Trp-Nle], Ac-Phe-[Orn-Pro-cha-Trp-Met], Ac-Phe-[Orn-Pro-cha-Trp-Nva], Ac-Phe-[Orn-Pro-cha-Trp-Hle], Ac-Phe-[Orn-Pro-cha-Trp-Eaf], Ac-Phe-[Orn-Pro-cha-Trp-Ebd], Ac-Phe-[Orn-Pro-cha-Trp-Eag], Ac-Phe-[Orn-Pro-cha-Trp-Pmf], Ac-Phe-[Orn-Pro-cha-Trp-2Ni], Ac-Phe-[Orn-Pro-cha-Trp-Thi], H-Phe-[Orn-Pro-cha-Trp-Nle], Ac-Phe-[Orn-Pro-cha-Trp-Nle], Ac-Lys-Phe-[Orn-Pro-cha-Trp-Nle], Ac-Phe-[Orn-Ser-cha-Trp-Phe], HOCH₂(CHOH)₄—C═N—O—CH₂—CO-Phe-[Orn-Pro-cha-Trp-Nle], Ac-Phe-[Orn-Hyp(COCH₂OCH₂CH₂OCH₂CH₂OCH₃)-cha-Trp-Phe], Ac-Phe-[Orn-Hyp(CONHCH₂COH(OH)CH₂OH)-cha-Trp-Phe], Phenylpropionyl-[Orn-Pro-cha-Trp-Nle], Ac-Phe-Orn-Pro-hle-Bta-Phe-NH₂, Ac-Phe-Arg(CH₂—CH₂)-Pro-cha-Bta-Phe-NH₂.

The oral absorption of peptides is influenced by a variety of factors like size, charge, and hydrophobicity. Nevertheless, the oral availability of a peptide cannot be predicted a priori. In general, peptides are regarded to have poor oral availability (Burton et al. 1996 Journal of Pharmaceutical Sciences 85: 1337-1340). A model for the estimation of the oral absorption is the measurement of the AB permeability through a monolayer of gut epithelial cells (e.g. CaCo2 or TC-7) (example 15, Lennernäs 1997 Journal of Pharmacy and Pharmacology 49: 627-38). The compounds according to the invention which can be used as C5aR antagonists, show a significantly increased AB permeability due to the hydrophobic substitution of the C-terminal arginine. For example, the antagonist Ac-Phe-[Orn-Hyp-cha-Trp-Phe] has a surprisingly high permeability of 14.3×10⁻⁶ cm/s compared to the bad permeability of 0.52×10⁻⁶ of the charged antagonist Ac-Phe-[Orn-Pro-cha-Trp-Arg]. The high permeability is in terms of figures within a range close to the one of compounds which are orally well available. An example for an orally well available compound is Propanolol which shows an AB permeability of 31.1×10⁻⁶ cm/s in this test by Lennemäs.

It is also within the present invention that, in an embodiment, the compounds according to the present invention have introduced groups at X1 and/or X4 which increase and preferably improve water solubility. Especially useful for improving water solubility is the introduction of groups which are able to have strong interactions with water and which are strongly solvatized. Frequently used groups are: hydroxy, keto, carboxamido, ether, urea, carbamate, amino, substituted amino, guanidino, pyridyl, carboxyl. The disclosed groups can explicitly be introduced at all positions at X1 and/or X4, and both one and several of the water solubility increasing groups can be introduced. Examples for the introduction of several groups are the attachments of carbohydrate residues and ethylene glycols.

Therefore, the present invention especially also includes peptidic and peptidomimetic C5aR antagonists, especially according to the present invention, the solubility of which is increased by additional modifications. Such modifications are known to the one skilled in the art and include, for example, the introduction of the previously mentioned solubility increasing groups. This is an efficient method and, respectively, leads to highly active antagonists as will be demonstrated by the following examples.

In accordance with example 13, compound 1 shows a solubility of 8% in aqueous HEPES buffer (pH 7.4). In contrast thereto, compound 40 has a solubility of 94% in HEPES buffer. Compound 2 which has an additional OH group compared to compound 1, shows a solubility of 13%. By adding more complex hydrophilic groups as shown for compound 4, the solubility is increased from 22% (compound 28) to 84% (compound 4). This is true although compound 4 is not charged. Thus it is ensured that the peptide and peptidomimetics according to the present invention, despite their hydrophobic character, can be converted into a well water-soluble form.

In the following some terms are set forth the meaning of which is to be used for embodiments of the present invention, in particular those which are set forth herein in more detail. Although these terms are occasionally referred to as definitions, the meaning of the various terms is not necessarily limited thereto.

The term “comprises” means, in preferred embodiments, that the respective structural element is included, but the structure is not limited to it.

The term “substituted” means, in preferred embodiments, that one or several hydrogen atoms of a group or a compound is/are replaced by a different atom, group of atoms, molecule or group of molecules. In connection therewith, such an atom, group of atoms, molecules and group of molecules itself/themselves is/are referred to as substituents or substitutions. A prerequisite for any substitution is that the normal valence of the atom is not exceeded, and that the substitution results in a stable compound. By the substitution of two hydrogen atoms a carbonyl group (C═O) can be generated. Carbonyl groups are preferably not present in aromatic moieties.

Substituents or substitutions can preferably be selected individually or in any combination from the group comprising hydroxyl, alkoxyl, mercapto, alkyl, alkenyl, alkynyl, alkoxy, alkylthio, alkylsulfinyl, cycloalkyl, heterocyclyl, aryl, arylalkyl, arylalkoxy, heteroaryl, aryloxy, halogen, trifluoromethyl, difluoromethyl, cyano, nitro, azido, amino, aminoalkyl, carboxamido, —C(O)H, acyl, oxyacyl, carboxyl, carbamate, trialkylsilyl, sulphonyl, sulfone amide and sulfuryl. Each substituent itself can be substituted further by one or several further substituents. This applies particularly to allyl, cycloalkyl, heterocyclyl, aryl, heteroaryl and aryloxy. Furthermore any definitions set forth herein may apply also to substituents.

The term “alkyl” refers, in an embodiment of the present invention, to a saturated aliphatic radical comprising from one to ten carbon atoms or a mono- or polyunsaturated aliphatic hydrocarbon radical comprising from two to twelve carbon atoms and at least one double and/or triple bond. The term “alkyl” includes both branched and unbranched alkyl groups. Unbranched alkyl groups having from one to eight carbon atoms, are preferred. Unbranched alkyl groups having from one to six carbon atoms and branched alkyl groups having from three to six carbon atoms are particularly preferred. It should be understood that the term “alkyl” comprises any analogs which can be put together from combination terms of the prefix “alk” or “alkyl”.

For example, the term “alkoxy” or “alkylthio” refers to an alkyl group which is linked by an oxygen or sulfur atom. “Alkanoyl” refers to an alkyl group which is linked by or to a carbonyl group (C═O).

The term “cycloalkyl” refers, in an embodiment of the present invention, to the cyclic derivatives of an alkyl group as defined above, which is optionally unsaturated and/or substituted. Saturated cycloalkyl groups are preferred, particularly those having from three to eight carbon atoms. Particularly preferred are cycloalkyl groups having three to six carbon atoms.

The term “aryl” refers, in an embodiment of the present invention, to an aromatic group having from 6 to 14 carbon atoms, whereby “substituted aryl” refers to aryl groups bearing one or more substituents.

Each of the above defined groups “alkyl”, “cycloalkyl”, and “aryl” comprise the respective halogenated derivatives, whereby the halogenated derivatives may comprise one or several halogen atoms. The halogenated derivatives comprise any halogen radical as defined in the following.

The term “halo” refers, in a preferred embodiment of the present invention, to a halogen radical selected from fluoro, chloro, bromo, and iodo. Preferred halo groups are fluoro, chloro and bromo.

The term “heteroaryl” refers, in an embodiment of the present invention, to a stable 5- to 8-membered, preferably 5- or 6-membered monocyclic or 8- to 11-membered bicyclic aromatic heterocyclic radical, whereby each heterocycle may consist of both carbon atoms and from 1 to 4 heteroatoms selected from the group comprising nitrogen, oxygen, and sulfur. The heterocycle may be linked by any atom of the cycle creating a stable structure. Within the present invention preferred heteroaryl radicals are, for example, furanyl, thienyl, pyrrolyl, oxazolyl, thiazolyl, imidazolyl, pyrazolyl, isoxazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzofuranyl, benzothienyl, indazolyl, benzimidazolyl, benzthiazolyl, benzoxazolyl, purinyl, quinolizinyl, quinolinyl, isoquinolinyl, cinnolinyl, phthalazinyl, quinazolinyl, quinoxalinyl, naphthridinyl, pteridinyl, carbazolyl, acridinyl, phenazinyl, phenothiazinyl and phenoxazinyl.

The term “heterocyclyl” refers, in an embodiment of the present invention, to a stable 5- to 8-membered, preferably 5- or 6-membered monocyclic or 8- to 11-membered bicyclic heterocyclic radical which may be either saturated or unsaturated, but is not aromatic. Each heterocycle comprises both carbon atoms and from 1 to 4 heteroatoms selected from the group comprising nitrogen, oxygen and sulfur. The heterocycle may be linked by any atom of the cycle, which results in a stable structure. Preferred heterocyclic radicals within the present invention include, for example, pyrrolinyl, pyrrolidinyl, pyrazolinyl, pyrazolidinyl, piperidinyl, morpholinyl, thiomorpholinyl, pyranyl, thiopyranyl, piperazinyl, indolinyl, azetidinyl, tetrahydropyranyl, tetrahydrothiopyranyl, tetrahydrofuranyl, hexahydropyrimidinyl, hexahydropyridazinyl, 2,5-dioxo-hexahydro-pyrimidin-4-yl, 2,6-dioxo-piperidin-4-yl, 2-oxo-hexahydro-pyrimidin-4-yl, 2,6-dioxo-hexahydro-pyrimidin-4-yl, 3,6-dioxo-piperazin-2-yl, 1,4,5,6-tetrahydropyrimidin-2-ylamine, dihydro-oxazolyl, 1,2-thiazinanyl-1,1-dioxide, 1,2,6-thiadiazinanyl-1,1-dioxide, isothiazolidinyl-1,1-dioxide and imidazolidinyl-2,4-dione.

When the terms “heterocyclyl”, “heteroaryl” and “aryl” are used together with other expressions and terms, the above definitions are further applicable. For example, “aroyl” refers to a phenyl or naphthyl group linked to a carbonyl group (C═O).

Each aryl or heteroaryl compound also includes its partially or fully hydrogenated derivatives. For example, quinolinyl may also include decahydroquinolinyl and tetrahydroquinolinyl naphthyl may also include the hydrogenated derivatives such as tetrahydronaphthyl.

Within the present invention the terms “nitrogen” or “N” and “sulfur” or “S” include any oxidized derivative of nitrogen like nitrones, N-oxides or of sulfur like sulfoxides, sulfones and the quaternized forms of any basic nitrogen like HCl or TFA salts.

Radicals can be any of mono-, di-, tri-, and tetra-radicals. Because of this it is possible that the meaning of various terms slightly changes. For example, a di-radical described as “propyl”, inevitably means “propyplene” (e.g. —(CH₂)₃—).

Any wording which specifies the limits of a range such as, e.g., “from 1 to 5” means any integer from 1 to 5, i.e. 1, 2, 3, 4 and 5. In other words, any range that is defined by two integers comprises both the two integers defining said limits of the definition and any integer comprised in said range.

The present invention also comprises all isotopes of atoms of the described compounds. Isotopes are atoms having the same atomic number but different mass numbers. For example, tritium and deuterium are isotopes of hydrogen. Examples for carbon isotopes are ¹¹C, ¹³ C and ¹⁴ C.

The term “energetically accessible conformer” means any conformer of a compound that falls within about a 20 kcal/mol window above the lowest energy conformation. In connection therewith, e.g., a Monte Carlo or systematic conformational search using MM2, MM3, or MMFF force fields as implemented in molecular modeling software such as MacroModel® v 7.0, Schrödinger Inc. Portland, Oreg., USA (http://www.schrodinger.com) or the like, can be used.

Amino acids are well-known to the ones skilled in the art and defined by the fact that a molecule contains both an amino and a carboxy group. Both natural and unnatural amino acids can be meant. Examples are α-, β-, and ω-amino acids, whereby preferably α-amino acids, more preferably E-L-amino-acids are used. In case an amino acid is not specified in more detail (e.g. “tryptophane”), both the L- and the D-form are meant.

A natural amino acid is an L-amino acid selected from the group comprising glycine, leucine, isoleucine, valine, alanine, phenylalanine, tyrosine, tryptophane, aspartic acid, asparagine, glutamic acid, glutamine, cysteine, methionine, arginine, lysine, proline, serine, threonine and histidine.

An unnatural amino acid is a non proteinogenic amino acid, which includes, but is not limited to, D-amino acids, N-alkyl-amino acids, homo amino acids, α,α-disubstituted amino acids and dehydro amino acids.

Amino acid derivatives are compounds which result from amino acids by modifying the N and/or C-terminus. Non-limiting examples are the conversion of the carboxy group to salts, esters, acylhydrazides, hydroxamic acids or amides, and the conversion of the amino group to amides, ureas, thioureas, thioamides, sulfonamides, phosphoric acid amides, boric acid amides or alkyl amines. Parts or moieties of compounds, which result from modifications of amino acids at the C and/or N-termini, can also be referred to as amino acid units. Furthermore, the amino acids can also be derivatized at their side chains. If a derivatized amino acid is an amino acid the side chain of which is derivatized one or several times, this kind of derivatization is usually specifically indicated herein. A preferred derivatisation of the side chain may be made in particular where the side chain bears a functional group. A preferred functional group is, for example, an amino group, a carboxyl group, a thiol group or an alcohol group.

Amino acid analogues are compounds, which result from amino acids by replacing the amino and/or carboxy group by other groups which can mimic them. Non-limiting examples are the incorporation of thioamides, ureas, thioureas, acylhydrazides, esters, alkyl amines, sulfonamides, phosphoric acid amides, ketones, alcohols, boronic acid amides, benzodiazepines and other aromatic or non-aromatic heterocycles (for a review see M. A. Estiarte, D. H. Rich in Burgers Medicinal Chemistry, 6th edition, volume 1, part 4, John Wiley & Sons, New York, 2002).

Aromatic amino acids are amino acids which contain aryl or heteroaryl groups. Non-limiting examples are phenylalanine, 2-fluoro-phenylalanine, 3-fluoro-phenylalanine, 4-fluoro-phenylalanine, 2-chloro-phenylalanine, 3-chloro-phenylalanine, 4-chloro-phenylalanine, tyrosine, histidine, tryptophane, homo-phenylalanine, homo-tyrosine, homo-histidine, homo-tryptophane, 1-naphthylalanine, 2-naphthylalanine, 2-thienylalanine, 3-thienylalanine, benzothienylalanine, furylalanine, thiazolylalanine, pyridylalanine, tetrahydroisochinoline-2-carboxylic acid, 2-aminoindane-2-carboxylic acid, biphenylalanine, 3,3-diphenylalanine and corresponding D- and β-amino acids.

Hydrophobic amino acids are amino acids, which contain hydrophobic alkyl-, cycloalkyl-, heterocyclyl, aryl or heteroaryl groups. Non-limiting examples are leucine, isoleucine, valine, phenylalanine, tyrosine, histidine, cysteine, cysteine(iPr), cysteine(tBu), methionine, proline, tryptophane, norleucine, norvaline, homoleucine, cyclohexyl alanine, cyclopentyl alanine, 1-naphthylalanine, 2-naphthylalanine, 2-thienylalanine, 3-thienylalanine, benzothienylalanine, allyl glycine, propargylglycine, 2-methyl-phenylalanine, 3-methyl-phenylalanine, 4-methyl-phenylalanine, homocyclohexylalanine, cyclohexyl glycine, n-cyclohexylglycine, octahydroindol-2-carboxylic acid and corresponding D- and β-amino acids.

The biological binding characteristics of an amino acid unit are those binding characteristics shown by the respective amino acid during the interaction with a biological molecule. Biological molecules are especially molecules exerting a biological function. Non-limiting examples of such biological molecules are protein- or peptide-based receptors.

Groups or units or moieties which mimic or imitate the biological binding characteristics of an amino acid, are defined as groups, which can establish with a receptor or interacting partner, preferably a biological receptor or a biological interaction partner, an interaction identical or similar to the amino acid itself. For the selection of such groups it is preferred to take into consideration those which are the most wide-spread ones in terms of most preferred interactions of the respective amino acids with biological receptors. For example, the oxygen atom of a carbonyl group of an amino acid can function as hydrogen bond acceptor, whereas the NH proton can establish interactions as hydrogen bond donor. Amino acids can additionally interact with receptors via their side chains. Phenylalanine and tryptophane can establish both hydrophobic interactions via the methylene side chain or the aromatic groups and πr-πr-interactions via the aromatic groups. Additionally, the indole group of the tryptophane can serve as a hydrogen bond donor via its NH group. Cyclohexyl alanine and norleucine can, in principle, establish hydrophobic interactions with biological receptors via their alkyl and/or cycloalkyl side chains. Not only the complete side chain of an amino acid, but also parts of the side chain can establish important interactions.

If a group, a unit or a moiety, which is to mimic or imitate the biological binding characteristics of an amino acid or shall exhibit this characteristic, is capable of establishing at least one of the above-mentioned interactions of the respective amino acid, then this group or unit can mimic its biological binding characteristics.

As used herein in connection with the definition of the groups, the term “and respective derivatives thereof” refers to the fact that all derivatives of the individual compounds, groups of compounds, parts or moieties of molecules, radicals or chemical groups as recited in the respective group, can each be present as derivatives.

As used herein the term “individually and independently” refers to the fact that the two or more substituents mentioned can be designed as described in the respective paragraph. The wording “individually and independently” shall only avoid unnecessary repetitions and discloses that any of the mentioned substituents can exhibit the described arrangement, whereby the arrangement for each substituent is made individually or is individually present and is not affected by the selection of one or several of the other substituents.

It is generally within the scope of the present invention that the substituents described for the individual compounds according to the invention, in particular for the generic structures, are also applicable to all of the generic formulas with the corresponding substituents, if not indicated to the contrary.

Spacers as used herein, are in preferred embodiments organic radicals having a molecular weight of approximately 1-300, which allow a covalent linkage between different chemical groups if not indicated to the contrary for the individual case. Examples are simple groups like

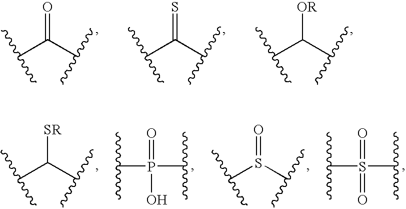

or more complex units like

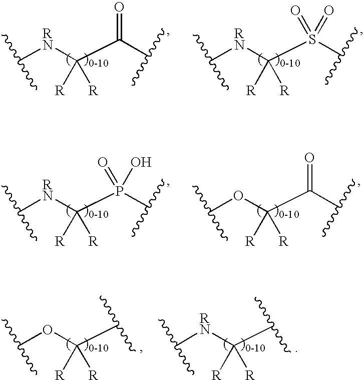

wherein R is, for each substitution, individually and independently a residue with a molecular weight of approximately 1-300. Preferably, R is a radical selected from the group comprising H, alkyl, substituted alkyl, cycloalkyl, substituted cycloalkyl, cycloalkylalkyl, substituted cycloalkylalkyl, heterocyclyl, substituted heterocyclyl, heterocyclylalkyl, substituted heterocyclylalkyl, aryl, substituted aryl, arylalkyl, substituted arylalkyl, heteroaryl, substituted heteroaryl, heteroaryl alkyl, substituted heteroarylalkyl, acyl, substituted acyl, alkoxyalkyl, substituted alkoxyalkyl, aryloxyalkyl, substituted aryloxyalkyl, sulfhydrylalkyl, substituted sulfhydrylalkyl, hydroxyalkyl, substituted hydroxyalkyl, carboxyalkyl, substituted carboxyalkyl, carboxamidoalkyl, substituted carboxamidoalkyl, carboxyhydrazinoalkyl, ureidoalkyl, aminoalkyl, substituted aminoalkyl, guanidinoalkyl and substituted guanidinoalkyl.

Spacers are preferably selected from the group comprising

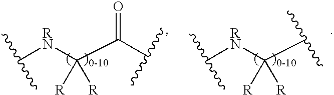

wherein R is preferably a radical selected from the group comprising H, alkyl, substituted alkyl, arylalkyl, substituted arylalkyl, heteroarylalkyl and substituted heteroarylalkyl.

Peptides carrying a positive net charge can cause a histamine release (Jasani et al. 1979 Biochemical Journal 181: 623-632). In particular subcutaneous administration and/or implantation of subcutaneous depots is not possible with such compounds. In case of orally administered drugs absorption of the drugs is particularly important. The absorption of charged molecules is usually inferior to the one of uncharged molecules under otherwise identical conditions (Veber et al. 2002 Journal of Medicinal Chemistry 45: 2615-2623). Due to the missing net charge of the compounds according to the present invention they are also suitable for use as oral drugs.

The compounds according to the present invention can be used for the manufacture of a medicament, in particular for the manufacture of a medicament for the prevention and/or treatment of immuno inflammatory and/or additionally for acute inflammatory diseases and/or local inflammations. In particular the following diseases belong to the group of immuno inflammatory diseases: Autoimmune diseases, acute inflammatory diseases, trauma, local inflammations, septic shock and hemorrhagic shock. In preferred embodiments these diseases are selected from the group consisting of septic shock, asthma, inflammatory diseases of the intestine (IBD: inflammatory bowel disease), pemphigus, glomerulonephritis, acute respiratory insufficiency, cerebral apoplexy, cardiac infarction, reperfusion injury, neurocognitive dysfunctions, burns, inflammatory diseases of the eye such as, e.g., uveitis, age related macular degeneration, diabetic retinopathy, local manifestations of systemic diseases such as rheumatoid arthritis, SLE, diabetes of the eye, the brain, the vasculature, the heart, the lung, the kidneys, the liver, the gastrointestinal tract, the spleen, the skin or other organ systems, inflammatory diseases of the vasculature e.g. vasculitis, arteriosclerosis, and acute injuries of the central nervous system. All these diseases and/or clinical characteristics are mainly derived from the group of immuno inflammatory and inflammatory diseases, respectively, whereby the inflammatory response of these diseases may be either the cause or a secondary reaction thereof.

Other diseases which can be treated with compounds according the present invention are autoimmune diseases, trauma, shock and burn injuries.

The compounds according to the present invention are also suitable for the treatment of auto-immune diseases since the C5a receptor is not only found on cells of the innate immune system but also on, e.g., T-cells. Additionally, it was shown that antigene presenting cells (APCs) which have the C5a receptor and which are stimulated by it release a modified set of cytokines. This leads, e.g., to a divergent differentiation of T cells into Th1 or Th2 cells. With this new aspect of influencing lymphocytes and APC with C5a inhibition—affecting lymphocyte mediated immune responses becomes possible. Such an approach allows for the treatment of a broad spectrum of autoimmune diseases which are difficult to treat otherwise. The group of autoimmune diseases comprises, e.g., the following diseases: Alopecia greata, cold agglutinin immunohemolytic anemia, warm antibody immunohemolytic anemia, pernicious anemia (Biermer's disease, Addison's anemia), antiphospholipid antibody syndrome (APS), arteriitis temporalis, atherosclerosis, autoimmune adrenalitis Addison's disease, chronic fatigue syndrome (CFIDS), chronic-inflammatory polyneuropathy, Churg-Strauss syndrome, Cogan's syndrome, ulcerative colitis, CREST syndrome, diabetes mellitus type I, dermatitis herpetiformis, dermatomyositis, fibromyalgia, chronic autoimmune gastritis, Goodpasture syndrome (anti-GBM antibody related glomerulonephritis), Guillain-Barré syndrome (GBS; Polyradiculoneuropathy), Hashimoto thyroiditis, autoimmune hepatitis, idiopathic pulmonary fibrosis, autoimmune thrombocytopenice purpura (Werlhof's disease), autoimmune infertility, autoimmune inner ear deafness (AIED), juvenile rheumatoid arthritis, autoimmune cardiomyopathy, Lambert-Eaton syndrome, Lichen sclerosis, Lupus erythematosus, Lyme arthritis, collagenosis, Graves' disease, Behcet disease, Crohn's disease, rheumatoid spondyliti, Méniére's disease, Reiter's disease, multiple sclerosis (MS, encephalomyelitis), myasthenia gravis, sympathetic ophtalmy, scaring pemphigoid, bullous pemphigoid, pemphigus vulgaris, polyarteritis nodosa, polychondritis, polyglandular autoimmune (PGA) syndrome, polymyalgia rheumatica, polymyositis, primary biliary cirrhosis, psoriasis, rheumatic fever, rheumatoid arthritis, sarcoidosis (Besnier-Boeck-Schaumann's disease), Sjörgen syndrome, scleroderma, sprue, stiff-man syndrome, systemic lupus erythematosus, Takayasu arteritis, transient gluten intolerance, autoimmune uveitis, vasculitis and vitiligo.

Vasculites are a group of different diseases, which can be treated or prevented with compounds according to the present invention. Vasculites can be seen as a special form of autoimmune diseases. In more detail: vasculites are different inflammatory diseases of the vessels. Primary and secondary vasculites are sub-groups of the vasculites. Primary vasculites are triggered by autoantibodies found in patients. One form of vasculites which can be treated with the compounds according to the present invention, are such vasculites which are triggered by cytoplasmatic anti-neutrophile antibodies (ANCA). This group comprises, e.g., Wegener's disease, Churg-Strauss syndrome und microscopic polyangiitis. Secondary vasculites are, e.g., drug-induced vasculites and vasculites which are induced through diseases like AIDS, hepatitis B or C, or cytomegalovirus infection.

In the course of the primary disease forms, leukoplastic vasculitis and/or tissue infiltration with eosinophils can occur (Churg-Strauss syndrome). The diseases are characterized by, e.g., a deposit of immune complexes and an activation of the complement system. Additionally, the autoantibodies against the neutrophils activate them, leading to the production and release of reactive oxygen. This leads additionally to a damage of, e.g., endothelial cells. Neutrophils and other leukocytes carry the C5a receptor and can be activated by binding of C5a. C5a is released during complement activation and is a strong proinflammatory agent.

Without therapy Wegner's disease can be fatal. Mostly patients die because af acute lung or renal failure. The current treatment thereof includes unspecific immune suppression with drugs like cyclophosphamide, glucocorticoids, methotrexate, mycophenolate mofetil, azathioprine or leflunomide. These therapies are associated with numerous side effects like increased infections, and decrease in white blood cell counts. Therefore a more targeted therapy for this indication is needed and can be provided by treatment with compounds according to the present invention.

For those skilled in the art it is easy to understand, that different diseases are summarized under certain terms or generic terms. These summaries are no limitation and each disease can be viewed on its own and can be treated or prevented with the compounds according to the present invention. Those skilled in the art will understand that the terms in paranthesis are synonyms or a special form of a certain disease.

The present invention is also related to formulations, in particular pharmaceutical formulations, which comprise at least one of the compounds according to the invention. Frequently pharmaceutically active compounds are combined with other pharmaceutically acceptable ingredients, in order to ensure an improved efficacy like improved transport, shelf-life, release behavior over time and the like. A variety of such appropriate formulations are known to the one skilled in the art. Ingredients of such formulations are, among others, inert diluents, calcium carbonate, sodium carbonate, lactose, calcium phosphate, sodium phosphate, starch, alginate, gelatine, magnesium stearate and talcum. Certain ingredients can be added, in order to allow for a retarded release of the pharmaceutically active compounds. Respective examples are glycerol monostearate and glycerol distearate. For oral application in particular hard gelatine capsules are used, whereby the pharmaceutically active ingredient is admixed with calcium carbonate, calcium phosphate or kaolin. For soft gelatine capsules the pharmaceutically active compounds are admixed with, e.g., oils (peanut oil, liquid paraffin, olive oil). For the application in aqueous solutions the pharmaceutically active ingredients can be admixed in particular with the following components: carboxymethylcellulose, methylcellulose, hydropropylmethylcellulose, sodium alginate, polyvinylpyrrolidone, lecithin, polymer products of alkylene oxides and fatty acids as for example polyoxyethylenestearate, heptadecaethyleneoxycetanol, polyoxyethylenesorbitol monooleate and polyoxyethylenesorbitane monooleate. For the purpose of preservation different additives may be used. Respective examples are ethyl or n-propyl-p-hydroxybenzoate.

Certain formulations are used in order to allow for particular routes of administration. Examples for routes of administration of compounds according to the present invention are oral, subcutaneous, intravenous, topical, intramuscular, rectal and inhalativ administration. The compounds according to the present invention can be present as pharmaceutical acceptable salts.

The invention is now further illustrated by reference to the following figures and examples from which further advantages, features and embodiments may be taken.

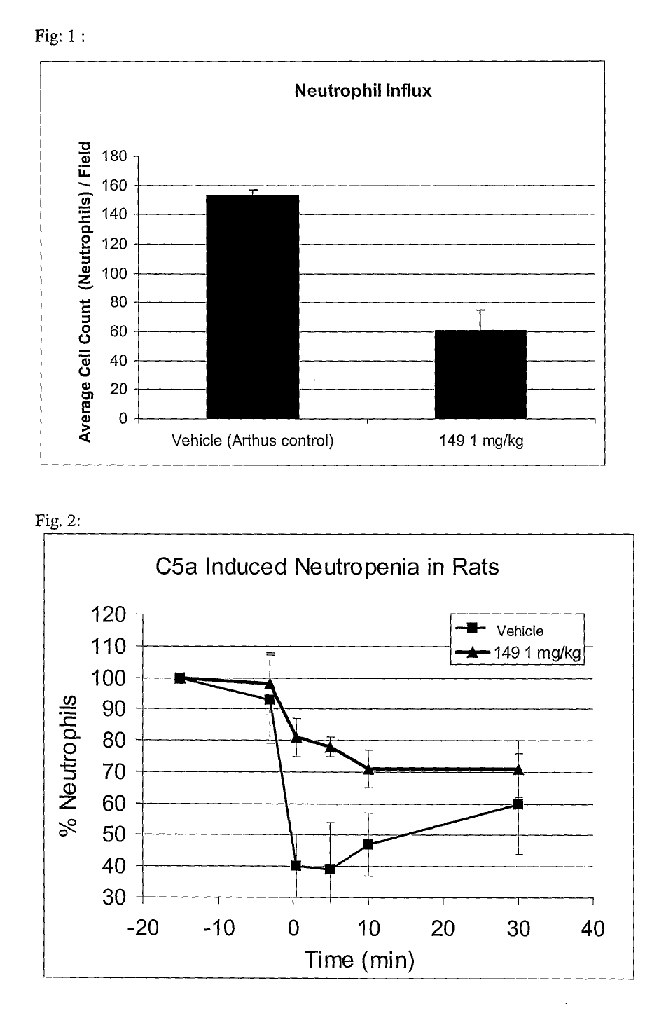

FIG. 1 shows a histogram indicating the influx of neutrophils in connection with immune complex mediated peritonitis expressed as average number of the polymorphonuclear cells/field, upon administration of compound 149 compared to the administration of the vehicle alone.

FIG. 2 shows a histogram indicating C5a-induced neutropenia in rats expressed as percentage of neutrophils over time, upon administration of compound 149 and of the vehicle alone, respectively.

### EXAMPLES

### Example 1

### Material and Methods

The materials and methods as well as general methods are further illustrated by the following examples:

### Solvents:

Solvents were used in the specified quality without further purification.

Acetonitrile (Gradient grade, J. T. Baker); dichloromethane (for synthesis, Merck Eurolab); diethylether (for synthesis, Merck Eurolab); N,N-dimethylformamide (LAB, Merck Eurolab); dioxane (for synthesis, Aldrich); methanol (for synthesis, Merck Eurolab).

Water: Milli-Q Plus, Millipore, demineralized.

### Reagents:

The used reagents were purchased from Advanced ChemTech (Bamberg, Germany), Sigma-Aldrich-Fluka (Deisenhofen, Germany), Bachem (Heidelberg, Germany), J. T. Baker (Phillipsburg, USA), Lancaster (Müihlheim/Main, Germany), Merck Eurolab (Darmstadt, Germany), Neosystem (Strassburg, France), Novabiochem (Bad Soden, Germany, from 2003 on Merck Biosciences, Darmstadt, Germany) und Acros (Geel, Belgium, distributor Fisher Scientific GmbH, Schwerte, Germany), Peptech (Cambridge, Mass., USA), Synthetech (Albany, Oreg., USA), Pharmacore (High Point, N.C., USA), Anaspec (San Jose, Calif., USA) and used in the specified quality without further purification.

Commercially available unnatural amino acids or carboxylic acids for N-terminal modification were prepared according to standard protocols. For example Fmoc-cis-Hyp-OH was prepared by the reaction of h-cis-Hyp-OH with Fmoc OSu [Paquet et al. 1982 Canadian Journal of Chemistry 60: 976-980A]. Fmoc-Phe(4-STrt-amidino)-OH was synthesized according to methods described in literature [Pearson et al. 1996 Journal of Medicinal Chemistry 39:1372-1382]. Side chain modified cysteine derivatives were prepared by alkylation of Fmoc cystein-OH with alkyl halides.

If not stated differently, concentrations are given as percent by volume.

RP-HPLC-MS analyses:

For analytic chromatography a Hewlett Packard 1100-system (degasser G1322A, quaternary pump G1311A, automatic sample changer G1313A, column heater G 1316A, variable UV detector G1314A) together with an ESI-MS (Finnigan LCQ ion trap mass spectrometer) was used. The system was controlled by “navigator ver. 1,1 sp1” software (Finnigan). Helium was used as impact gas in the ion trap. For chromatographic separation a RP-18-column (Vydac 218 TP5215, 2.1×150 mm, 5 μm, C18, 300 A with a pre column (Merck)) at 30° C. and a flow of 0.3 ml/min using a linear gradient for all chromatograms (5-95% B for 25 min, linear, A: 0.05% TFA in water and B: 0.05% TFA in CH₃CN) was used. UV detection was at λ=220 nm. Retention times (Rₜ) are indicated in the decimal system (e.g. 1.9 min=1 min 54 s) and are referring to detection in the mass spectrometer. The dead time between injection and UV detection (HPLC) was 1.65 min, and between UV detection and mass detection 0.21 min. The accuracy of the mass spectrometer was approx. ±0.2 amu.

Analyses by means of HPLC/MS was performed by injection of 5 μl, using a linear gradient from 95:5 to 5:95 in 9.5 min (A: 0.05% TFA in water and B: 0.05% TFA in acetonitrile), RP columns were from the company Phenomenex, (Typ Luna C-18, 3 μm, 50×2.00 mm, flow 0.3 ml, HPLC at room temperature); Mass spectrometer: ThermoFinnigan Advantage and/or LCQ Classic (both iontrap), ESI ionization, helium served as impact gas in the ion trap. Excalibur vers. 1.3 and/or. 1.2 was used as software. Retention times (Rₜ) are indicated in the decimal system (e.g. 1.9 min=1 min 54 s).

### Preparative HPLC:

Preparative HPLC separations were performed using Vydac R18-RP columns with the following gradient solvents: 0.05% TFA in H₂O and B: 0.05% TFA in CH₃CN

TABLE 1
Abbreviations:

| | |
|---|---|
| AAV | general procedure |
| Ac | acetyl |
| Acm | acetamidomethyl |
| Ac | acetyl |
| d | doublet |
| DCM | dichloromethane |
| DIC | diisopropylcarbodiimide |
| DIPEA | N,N-diisopropylethylamine |
| DMF | N,N-dimethylformamide |
| DMEM | Dulbecco's modified eagle medium |
| DMSO | dimethylsulfoxide |
| eq. | equivalent(s) |
| Fig. | figure |
| Fmoc | 9-fluorenylmethyloxycarbonyl |
| h | hour(s) |
| HATU | O-(7-azabenzotriazol-1-yl)-1,1,3,3-tetramethyluronium- |
| | hexafluorophosphate |
| HBTU | O-(benzotriazol-1-yl)-1,1,3,3-tetramethyluronium- |
| | hexafluorophosphate |
| HEPES | N-2-2-hydroxyethyl-1-piperazine-N′-2-ethanesulfonic acid |
| HOBt | 1-hydroxybenzotriazole |
| HPLC | high-pressure liquid chromatography |
| m | multiplet |
| Me | methyl |
| min | minute(s) |
| ml | milliliter |
| NMI | N-methylimidazole |
| NMP | N-methylpyrrolidone |
| NMR | nuclear magnetic resonance |
| Ph | phenyl |
| s | singlet |
| ^{t}Bu | tert-butyl |
| THF | tetrahydrofuran |
| TFA | trifluoroacetic acid |

TABLE 2
For proteinogenic amino acids the 3-letter codes were used:

| 3-letter codes | Amino acids | 3-letter codes | Amino acids |
|---|---|---|---|
| Ala | Alanine | Met | Methionine |
| Cys | Cysteine | Asn | Asparagine |
| Asp | Aspartic acid | Pro | Proline |
| Glu | Glutamic acid | Gln | Glutamine |
| Phe | Phenylalanine | Arg | Arginine |
| Gly | Glycine | Ser | Serine |
| His | Histidine | Thr | Threonine |
| Ile | Isoleucine | Val | Valine |
| Lys | Lysine | Trp | Tryptophane |
| Leu | Leucine | Tyr | Tyrosine |

For non-proteinogenic amino acids a 3-letter code was used where the first letter indicates the stereochemistry of the C-alpha-atom. A capital first letter stands for the L-form, a lower case first letter stands for the D-form of the correspondent amino acid.

TABLE 3

| | |
|---|---|
| 1Ni | 1-naphthylalanine |
| 2Ni | 2-naphthylalanine |
| 3PP | 3-phenylpropionyl |
| 5Ff | pentafluorophenylalanine |
| 6FW | 6-fluoro-DL-tryptophane |
| Aic | 2-aminoindan-2-carboxylic acid |
| Amf | alpha-methyl-phenylalanine |
| Aoa | aminooxyacetic acid |
| Aoc | 1-aza-bicyclo-[3.3.0]-octan-2-carboxylic acid |
| Aze | azetidin-2-carboxylic acid |
| Bal | beta-alanine |
| Bhf | beta-homophenylalanine |
| Bta | benzothienylalanine |
| Bzl | benzyl |
| Cha | beta-cyclohexylalanine |
| Chg | cyclohexylglycine |
| Chy | cis-hydroxyproline |
| Cit | citrulline |
| Ctb | Cys(tBu) |
| Dab | 2,4-diaminobutyric acid |
| Dap | 2,3-diaminopropionic acid |
| Def | N,N-diethyl-phenylalanine |
| Dff | Phe(3,4-F) |
| Eaa | Phe(3,4-Cl) |
| Eaf | allylglycine |
| Eag | 2-propargylglycine |
| Eap | Phe(4-tBu) |
| Eay | (2S,4S)-4-phenyl-pyrrolidine-2-carboxylic acid |
| Ebd | Cys(Et) |
| Ebo | Cys(4-picolyl) |
| Ebu | Cys(3-picolyl) |
| Ebw | 3,3-diphenylalanine |
| Eby | (S)-3-amino-3-phenylpropanic acid |
| Ecf | Cys(O-3-picolyl) |
| Ecg | Cys(2-picolyl) |
| Ecp | His(tau-4-Methoxybenzyl) |
| Ecr | His(tau-methyl) |
| Edn | Cys(CH₂—CH₂-4-Pyridyl) |
| Eec | Cys(1-methylene-1H-benzotriazole) |
| Eep | Cys(O2-Acm); 3-(acetylamino-methanesulfonyl)-2- |
| | amino-propionic acid |
| Eew | Arg(NO₂) |
| Egc | DL-Trp(5-Me) |
| Egy | Phe(2,4-Cl) |
| Egz | Phe(3-NO₂) |
| Eth | ethyl |
| FAc | F—CH₂—CO— |
| Fai | —CONH₂ |
| Faz | 3-phenylpropionyl |
| Fbi | 2-(4-pyridyl)acetyl |
| Fbn | nicotinoyl |
| Fbo | morpholine-4-carbonyl |
| Fbp | N,N-dimethyl-phenylalanine |
| Fci | piperidine-3-carbonyl |
| Fck | HO—CH₂—(CHOH)₄—C═N—O—CH₂—CO— |
| Fcn | norArg(CH₂CH₂); 2-amino-4-(4,5-dihydro-1H-imidazol- |
| | 2-ylamino)-butyric acid |
| Fco | bisnorArg(CH₂CH₂); 2-amino-3-(4,5-dihydro-1H-imidazol- |
| | 2-ylamino)-propionic acid |
| Fcp | 2-amino-5-[bis-(4,5-dihydro-1H-imidazol-2-yl)-amino]- |
| | pentanoic acid |
| Ffa | Arg(CH₂CH₂); 2-amino-5-(4,5-dihydro-1H-imidazol- |
| | 2-ylamino)-pentanoic acid |
| Fha | 2-morpholine-4-yl-acetyl |
| Fhb | N-(2,3-dihydroxy-propyl)-formamidyl |
| Fhi | 2-[2-(2-methoxy-ethoxy)-ethoxy]-acetyl |
| Fhu | —C(NH)-NH₂ |
| Fib | Arg(4xMe), [(4-amino-4-carboxy-butylamino)-dimethylamino- |
| | methylene]-dimethyl-ammonium |
| Fid | methoxyoxalyl |
| G23 | Orn(SO₂Me) |
| G24 | N-(n-propyl)-glycine |
| G25 | N—(CH₂CH₂OCH₃)-glycine |
| G26 | N—(CH₂furyl)-glycine |
| G27 | N—(CH₂pyridyl)-glycine |
| G30 | N—(CH₂CH₂CH₂(2-oxo-pyrrolidin-1-yl))-glycine |
| G31 | N—(CH₂CH₂(3,4-dimethoxyphenyl))-glycine |
| Guf | Phe(4-guanidine) |
| Har | homo-arginine |
| Hch | homo-cyclohexylalanine |
| Hci | homo-citrulline |
| Hle | homo-leucine |
| Hoo | hydroorotic acid; (S)-2,6-dioxo-hexahydro-pyrimidine-4- |
| | carbonyl |
| Hse | homo-serine |
| HyA | Hyp(Ac) |
| Hym | Hyp(Me) |
| Hyp | trans-hydroxyproline |
| L19 | 1-(methoxymethyl)-2-phenyl-ethylamino |
| L22 | norArg |
| Mcf | Phe(3-Cl) |
| Mff | Phe(3-F) |
| Mmf | Phe(3-Me) |
| Mmy | Phe(3-OMe) |
| Mpa | 3-(3-pyridyl)-alanine |
| Nip | nipecotic acid |
| Nle | norleucine |
| NMA | N—Me-alanine |
| NMD | N—Me-asparagine |
| NMF | N—Me-phenylalanine |
| NMS | N—Me-serine |
| Nva | Norvaline |
| Ocf | Phe(2-Cl) |
| Off | Phe(2-F) |
| Ohf | (S)-2-hdroxy-3-phenyl-propionyl |
| Oic | octahydroindole-2-carboxylic acid |
| Omf | Phe(2-Me) |
| Opa | 3-(2-pyridyl)-alanine |
| OrA | Orn(Ac) |
| OrE | Orn(Et₂); 2-amino-5-diethylamino-pentanoic acid |
| Orn | ornithine |
| Otf | Phe(2-CF3) |
| Paf | Phe(4-NH₂) |
| Pcf | Phe(4-Cl) |
| Pff | Phe(4-F) |
| Phg | phenylglycine |
| Pip | pipecolinic acid |
| Pmf | Phe(4-Me) |
| Ppa | 3-(4-pyridyl)-alanine |
| Tff | Phe(3,4,5-F) |
| Thi | 2-thienylalanine |
| Tic | 1,2,3,4-tetrahydroisochinolin-3-carboxylic acid |
| Tiq | tetrahydroisochinoline-1-carbonxylic acid |
| Tmg | ═C(NMe₂)—NMe₂ |
| XX1 | 2-amino-3-(4-piperidinyl)propionic acid |
| XX2 | 4-guanidyl-piperidinyl-alanine |

The activity of the compounds was categorized based upon the following conventions:

| | |
|---|---|
| IC₅₀ < 5 nM: | A |
| 5 nM < IC₅₀ ≦ 10 nM: | B |
| 10 nM < IC₅₀ ≦ 20 nM: | C |
| 20 nM < IC₅₀ ≦ 50 nM: | D |
| 50 nM < IC₅₀ ≦ 200 nM: | E |
| 200 nM < IC₅₀ ≦ 2000 nM: | F |
| 2000 nM < IC₅₀ | G |

General procedure (AAV) 1: Synthesis of linear peptides

Linear peptides were synthesized using the Fmoc-tBu-strategy in batch-mode. The synthesis was done either manually in polypropylene syringes or via an automatic synthesizer (Syro from Multisyntech, Witten or Sophas from Zinsser, Frankfurt).

For the preparation of peptides carrying a C-terminal carboxylic acid, the C-terminal amino acid was either attached to a tritylchloride resin (approx. 200 mg resin; loading of reactive groups approx. 1.5 mmol/g; coupling with 0.8 eq. Fmoc-amino acid and 3.0 eq. DIPEA in CH₂Cl₂ for 2 h; loading of the first amino acid approx. 0.2-0.4 mmol/g) or to Wang resin (200-500 mg resin; loading of reactive groups approx. 0.6 mmol/g; coupling with 4 eq. Fmoc-amino acid, 4 eq. DIC and 3 eq. NMI in DMF for 3 h; loading of the first amino acid approx. 0.2-0.6 mmol/g).

For the preparation of peptides carrying a C-terminal carboxylic amide, the first amino acid was attached to the resin via Fmoc deprotection of the Fmoc-Rink amide resin (ca. 200 mg resin; Fmoc deprotection with 20% piperidine in DMF for 20 min) and subsequent coupling of the Fmoc amino acid (reaction with 5 eq. Fmoc amino acid; 5 eq. HBTU and 15 eq. DIPEA in DMF for 30-60 min repeated once or several times).

After the coupling of the first amino acid, the synthesis of the peptide was done via a repeated sequence of steps, as necessary, consisting of Fmoc deprotection and coupling of the corresponding Fmoc amino acid or carboxylic acids. For the Fmoc deprotection the resin was reacted with 20% piperidine in DMF for 20 min. The coupling of the amino acids was carried out via reaction, repeated once or more times, with 5 eq. of the amino acid, 5 eq. HBTU and 15 eq. DIPEA in DMF for 30-60 min. For the introduction of the N-terminal acetyl group, the N-terminal free peptide, bound to the resin, was incubated with a solution of 10% acetic acid anhydride and 20% DIPEA in DMF for 20 min.

For the cleavage of the peptide from the resin and cleavage of the side chain protecting groups, a mixture of 95% TFA, 2.5% H₂O, 2.5% TIPS or a similar solution was added. Finally, TFA was evaporated using a rotary evaporator or the peptide was precipitated by adding methyl-butyl-ether at 0° C. and isolated by centrifugation or decantation of the remaining solution. For transformation of the eventually formed TFA-salts in the correspondent HCl-salts, the peptide was dissolved in 2 N HCl and MeCN and lyophilized.

Peptides with C-terminal carboxylic amides were purified directly via HPLC. Peptides carrying C-terminal carboxylic acids as raw product were cyclized following AAV2.

General procedure (AAV) 2: Cyclization of peptides carrying a C-terminal carboxylic acid

For the cyclization approx. 80 mg of the linear peptide, synthesized following AAV1, were solubilized in 5 ml DMF and 5 ml CH₂Cl₂. Subsequently, the pH was adjusted to approx. 8 by adding N-ethylmorpholine followed by 1 eq. HOBt together with 10 eq. DIC. After 2-16 h of stirring at room temperature the solvent was evaporated and the raw product purified via HPLC.

General procedure (AAV) 3: Reductive alkylation of resin-bound peptides having a free N-terminus

Linear peptides, synthesized following AAV1, with a free N-terminus were incubated, before cleavage from the resin, with 10 eq. of the correspondent aldehyde in 5% acetic acid and 5% trimethylorthoformiate in THF. After approx. 4 h the resulting imine was reduced overnight with 5 eq. sodium cyanoborhydride.

After cleavage from the resin of the peptide synthesized following AAV1, the raw product was cyclized following AAV2. Frequently an undesired byproduct is observed due to cyclization via the secondary N-terminal amine. This side product can be removed by HPLC easily.

### Example 2

### Synthesis of Ac-Phe-[Orn-Pro-cha-Trp-Phe](1)

After linear peptide synthesis, following AAV1, cyclization, following AAV2, and subsequent purification via HPLC, 50.9 mg of the desired product Ac-Phe-[Orn-Pro-cha-Trp-Phe] were obtained as white solid.

MS (ESI): m/z=888.3 [(M+H)⁺].

### Example 3

### Synthesis of Ac-Phe-[Orn-Hyp-cha-Trp-Phe](2)

The linear peptide Ac-Phe-Orn-Hyp-cha-Trp-Phe-OH was obtained following AAV1 and cyclized following AAV2. Due to the higher nucleophilicity of amines compared to alcohols, no byproduct was observed, together with the desired cyclized product, through coupling of the free Hyp-OH group with the C-terminal carboxylic acid. Purification of the obtained raw product via HPLC yielded 26.9 mg of the desired white solid Ac-Phe-[Orn-Hyp-cha-Trp-Phe] (2).

MS (ESI): m/z=903.5 [(M+H)⁺].

### Example 4

### Synthesis of Ph-CH₂-[Orn-Pro-cha-Trp-Nle](56)

The resin-bound peptide H-Orn-Pro-cha-Trp-Nle-trityl-resin was obtained following AAV1 and subsequently it was incubated with benzaldehyde under reductive alkylation conditions. The cyclization following AAV2, and subsequent purification via HPLC yielded 0.9 mg of the desired product 56 as white solid.

MS (ESI): m/z=753.4 [(M+H)⁺].

### Example 5

### Synthesis of HOCH₂(CHOH)₄—C═N—O—CH₂—CO-Phe-[Orn-Pro-cha-Trp-Nle] (3)

The linear peptide H-Aoa-Phe-Orn-Pro-cha-Trp-Nle-OH was obtained following AAV1. It was dissolved in 24 ml MeCN/sodium acetate buffer (0.2 M, pH=4) 1:1 and incubated with 58 mg (10 eq.) D-glucose. After stirring for 5 days, 2.4 ml acetone were added for quenching the unconverted aminooxyacetic acid-peptide, and after 5 min the solvent was evaporated. The obtained raw product was purified via HPLC and subsequently cyclized following AAV2. The purification via HPLC of the raw product yielded 1.9 mg of the desired white solid 3.

MS (ESI): m/z=1046.5 [(M+H)⁺].

### Example 6

### Synthesis of 2-acetamido-1-methyl-glucuronyl-Phe-[Orn-Pro-cha-Trp-Nle] (4)

The resin-bound peptide H-Phe-Orn-Pro-cha-Trp-Nle-trityl-resin, which was obtained following AAV1, was incubated with 39.8 mg (2.0 eq.) 2-acetamido-1-methyl-glucuronic acid (Schämrann et al. 2003 European Journal of Organic Chemistry: 351-358), 60.8 mg (2.0 eq.) HATU and 105.7 μl (10 eq.) 2,4,6-collidine in 1.6 ml DMF. After stirring for 1.5 h the resin was washed with DMF (5×), MeOH (5×) und CH₂Cl₂ (3×) and the peptide was cleaved from the resin using 95% TFA, 2.5% H₂O and 2.5% TIPS. Cyclization following AAV2, and HPLC purification yielded 29.0 mg of the desired product 4 as white solid.

MS (ESI): m/z=1043.0 [(M+H)⁺].

### Example 7

### Synthesis of Ac-Phe-[Orn-Hyp(COCH₂OCH₂CH₂OCH₂CH₂OCH₃)-cha-Trp-Nle](5)

The linear peptide Ac-Phe-Orn-Hyp-cha-Trp-Nle-OH which was obtained following AAV1, was cyclized following AAV2 and the resulting cyclic peptide Ac-Phe-[Orn-Hyp-cha-Trp-Nle] was purified via HPLC. 35.4 μl (40 eq.) 2-(2-(2-methoxyethoxy)ethoxy)acetic acid were incubated for 15 min at 40° C. with 50.3 μl (120 eq.) thionyl chloride. After evaporation of the solvent, 78.8 ml (80 eq.) DIPEA, 1 ml CH₂Cl₂ and 5.0 mg of the compound Ac-Phe-[Orn-Hyp-cha-Trp-Nle] were added. Stirring was continued for 3 days at room temperature and purification was done via HPLC yielding 1.6 mg of the desired white solid 5.

MS (ESI): m/z=1029.6 [(M+H)⁺].

### Example 8

### Synthesis of Ac-Phe-[Orn-Hyp(CONH—CH₂CH(OH)—CH₂OH)-cha-Trp-Nle] (6)

The linear peptide Ac-Phe-Orn-Hyp-cha-Trp-Nle-OH was synthesized following AAV1, cyclized following AAV2, and the resulting cyclic peptide Ac-Phe-[Orn-Hyp-cha-Trp-Nle] was purified via HPLC. Subsequently, 5.0 mg of the peptide were incubated with 26.1 mg 4-isocyanatomethyl-2,2-dimethyl-[1,3]dioxolane and 1.88 μl (2.0 eq.) DIPEA in 0.3 ml MeCN. After stirring for 3 days at 40° C., the solvent was evaporated and the resulting raw product was purified via HPLC. 0.22 mg of the desired white solid 6 were obtained.

MS (ESI): m/z=986.5 [(M+H)⁺].

### Example 9

### Synthesis of Ac-Phe-[Orn-Pro-cha-Trp-Arg(CH₂CH₂)](7)

The linear peptide Ac-Phe-Orn-Pro-cha-Trp-Orn-OH was synthesized following AAV1, cyclized following AAV2, and the resulting cyclic peptide Ac-Phe-[Orn-Pro-cha-Trp-Orn] was purified via HPLC. Subsequently, 2.6 mg of the peptide were incubated with 22.6 mg (30 eq.) 2-(methylmercapto)-2-imidazoline-hydroiodide and 29.7 μl (60 eq.) DIPEA in 260 μl MeOH. After stirring for 2 days at 50° C., the solvent was evaporated and the resulting raw product was purified via HPLC. 0.86 mg of the desired white solid 7 were obtained.

MS (ESI): m/z=922.8 [(M+H)⁺].

### Example 10

### Synthesis of Ph-CH₂—CH₂—CO—[Orn-Pro-cha-Trp-Nle](41)

The peptide Ph-CH₂—CH₂—CO-Orn-Pro-cha-Trp-Nle-OH carrying 3-phenylpropionic acid as N-terminal carboxylic acid was synthesized following AAV1. The linear peptide was cyclized following AAV2 and the raw product was purified via HPLC. 3.13 mg of the desired white solid 41 were obtained.

MS (ESI): m/z=796.5 [(M+H)⁺].

### Example 11

### Determination of the IC₅₀ value in an enzyme release assay

The assay procedure was described by Kohl (Kohl 1997 The Anaphylatoxins. In: Dodds, A. W., Sim, R. B. (Eds.), Complement: A Practical Approach. Oxford, pp. 135-163). Basophilic leukemia cells from rats (RBL), which express the human C5aR(CD88), were cultivated in DMEM with 10% fetal bovine serum, 100 U/ml penicillin, 100 μg/ml streptomycin and 2 mM glutamine (all components of the media from Biochrome, Berlin) to confluence at 37° C. and 10% CO₂. The following specifications refer to single cell culture flask having a surface of 75 cm². Medium was decanted from cells. Cells were washed with 10 ml PBS (Dulbecco's PBS, Biochrome) and subsequently overlayed with 3 ml Cell Dissociation Solution (CDS, Sigma). Cells were incubated for 1 min at room temperature. Subsequently, CDS was removed and the cells were further incubated for 10-15 min at 37° C. for dissociation. In the assay, 20 μl of the solution containing the compound to be tested were used. This solution should not contain more than 2.8% DMSO. For the dilution process, the compounds were diluted in ⅓ or ½ steps. To 20 μl compound solution 75 μl of the RBL-cells were added, which were treated as follows: after dissociation cells were detached and warmed at 37° C. in 10 ml HAG-CM (20 mM HEPES; 125 mM NaCl, 5 mM KCl, 1 mM CaCl₂, 1 mM MgCl₂, 0,5 mM Glucose, 0,25% BSA. HEPES-preparation: 2.3 g/l HEPES-salt +2.66 g/l HEPES acid). Cells were counted and centrifuged (200×g, 10 min). The cell pellet was resuspended in preheated HAG-CM (i.e. Hepes-buffer NaCl-glucose-solution with calcium and magnesium), and the cell density was adjusted to 2×10⁶ cells/ml. The cells were incubated at 37° C. for 5 min. Per ml cell suspension 27 μl of a cytochalasin B-solution were added (100 μg/ml in DMSO, Sigma). The cells were incubated for further 3 min at 37° C. 75 μl of the cell suspension were added to 20 μl compound solution leading to a volume of 95 μl per well. After incubation of the cells for 10 min at 37° C. 10 μl hrC5a (10.5 nM in HAG-CM, Sigma) per well were added. The cells were incubated for additional 5 min at 37° C. The mtp was then transferred to ice and centrifuged for 3 min at 4° C. with 1200×g. 75 μl of the supernatant were added to 100 μl substrate-solution (2.7 mg/ml p-nitrophenyl-N-acetyl-β-D-glucosaminide (Sigma) in 42.5 mM Na-acetate pH 4.5). The mtp was incubated for further 60 min at 37° C. 75 μl 0.4 M glycine pH 10.4 were added per well. The mtp was transferred into a reader and the absorption at 405 nm was measured. IC₅₀-values were obtained following the equation; y=((A-D)/(1+(x/C)^{B}))+D.

The results of the IC50-value determination are shown in table 4.

TABLE 4
Data for antagonistic activity of selected compounds acording to the present invention.

| | | (M + H)⁺ in | activity |
|---|---|---|---|
| No. | Compound | MS [amu] | (group) |
| 1 | Ac-Phe-[Orn-Pro-cha-Trp-Phe] | 888.3 | D |
| 2 | Ac-Phe-[Orn-Hyp-cha-Trp-Phe] | 903.5 | D |
| 3 | HOCH₂(CHOH)₄—C═N—O—CH₂—CO-Phe-[Orn-Pro-cha-Trp- | 1046.5 | E |
| | Nle] | | |
| 4 | X-Phe-[Orn-Pro-cha-Trp-Nle]; X = 2-acetamido-1-methyl- | 1043.0 | D |
| | glucuronyl | | |
| 5 | Ac-Phe-[Orn-Hyp(COCH₂OCH₂CH₂OCH₂CH₂OCH₃)-cha- | 1029.6 | E |
| | Trp-Nle] | | |
| 6 | Ac-Phe-[Orn-Hyp(CONH—CH₂CH(OH)—CH₂OH)-cha-Trp- | 986.5 | E |
| | Nle] | | |
| 7 | Ac-Phe-[Orn-Pro-cha-Trp-Arg(CH₂CH₂)] | 922.8 | F |
| 8 | Ac-Phe-[Orn-Pro-cha-Trp-Har] | 910.7 | F |
| 9 | Ac-Phe-[Orn-Pro-cha-Trp-Guf] | 944.6 | F |
| 10 | Ac-Phe-[Orn-Pro-cha-Trp-Cit] | 897.5 | F |
| 11 | Ac-Phe-[Orn-Pro-cha-Trp-Eew] | 941.5 | F |
| 12 | Ac-Phe-[Orn-Pro-cha-Trp-arg] | 896.7 | F |
| 13 | Ac-Phe-[Orn-Pro-cha-Trp-Hci] | 911.6 | F |
| 14 | Ac-Phe-[Orn-Pro-cha-Trp-Paf] | 902.7 | D |
| 15 | Ac-Phe-[Orn-Pro-cha-Trp-Ebo] | 934.6 | F |
| 16 | Ac-Phe-[Orn-Pro-cha-Trp-Ecf] | 950.6 | F |
| 17 | Ac-Phe-[Orn-Pro-cha-Trp-Ebu] | 934.7 | F |
| 18 | Ac-Phe-[Orn-Pro-cha-Trp-Ecg] | 934.6 | F |
| 19 | Ac-Phe-[Orn-Pro-cha-Trp-Edn] | 948.6 | F |
| 20 | Ac-Phe-[Orn-Pro-cha-Trp-Ecr] | 891.7 | E |
| 21 | Ac-Phe-[Orn-Pro-cha-Trp-Phe(4-Amidin)] | 929.7 | F |
| 22 | Ac-Phe-[Orn-Pro-cha-Trp-Lys] | 868.6 | G |
| 23 | Ac-Phe-[Orn-Pro-cha-Trp-Ppa] | 888.6 | E |
| 24 | Ac-Phe-[Orn-Pro-cha-Trp-Arg(Me2)] | 924.7 | E |
| 25 | Ac-Phe-[Orn-Pro-cha-Trp-Dab] | 840.4 | E |
| 26 | Ac-Phe-[Orn-Pro-cha-Trp-Ecp] | 997.7 | F |
| 27 | Ac-Phe-[Orn-Pro-cha-Trp-XX1] | 894.6 | G |
| 28 | Ac-Phe-[Orn-Pro-cha-Trp-Nle] | 852.6 | D |
| 29 | Ac-Phe-[Orn-Pro-cha-Trp-Met] | 871.6 | E |
| 30 | Ac-Phe-[Orn-Pro-cha-Trp-XX2] | 936.5 | G |
| 31 | Ac-Phe-[Orn-Pro-cha-Trp-Nva] | 839.5 | C |
| 32 | Ac-Phe-[Orn-Pro-cha-Trp-Hle] | 867.5 | D |
| 33 | Ac-Phe-[Orn-Pro-cha-Trp-Eaf] | 837.5 | B |
| 34 | Ac-Phe-[Orn-Pro-cha-Trp-Ebd] | 871.5 | D |
| 35 | Ac-Phe-[Orn-Pro-cha-Trp-Eag] | 835.5 | B |
| 36 | Ac-Phe-[Orn-Pro-cha-Trp-Pmf] | 901.6 | D |
| 37 | Ac-Phe-[Orn-Pro-cha-Trp-2Ni] | 937.5 | E |
| 38 | Ac-Phe-[Orn-Pro-cha-Trp-Thi] | 893.5 | D |
| 39 | Ac-Phe-[Orn-Pro-cha-Trp-Ala] | 811.7 | G |
| 40 | Ac-Phe-[Orn-Pro-cha-Trp-Arg] | 896.6 | C |
| 41 | Ph-CH2—CH₂—CO-[Orn-Pro-cha-Trp-Nle] | 796.5 | C |
| 42 | H-Phe-[Orn-Pro-cha-Trp-Nle] | 811.5 | C |
| 43 | Ac-Lys-Phe-[Orn-Pro-cha-Trp-Nle] | 1015.7 | D |
| 44 | H-Phe-[Orn-Ser-cha-Trp-Nle] | 843.5 | D |
| 45 | Ac-Ala-[Orn-Pro-cha-Trp-Arg] | 820.6 | G |
| 46 | Ac-Phe-[Orn-NMeAla-cha-Trp-Arg] | 884.8 | D |
| 47 | Ac-Phe-[Orn-Pro-ala-Trp-Arg] | 814.8 | G |
| 48 | Ac-Phe-[Orn-Pro-cha-Ala-Arg] | 781.8 | G |
| 49 | Ac-Phe-[Orn-Pro-cha-Trp-Ala] | 811.7 | G |
| 56 | Ph-CH2-[Orn-Pro-cha-Trp-Nle] | 753.4 | D |
| 57 | Ph-CH2-[Orn-Pro-cha-Trp-Phe] | 787.5 | D |
| 58 | Ac-Phe-[Orn-Pro-cha-Trp-1Ni] | 937.7 | D |
| 59 | Ph-CH(OH)—CH₂—CO-[Orn-Pro-cha-Trp-Nle] | 812.4 | D |
| 144 | Ac-Phe-[Orn-Hyp-cha-Trp-Nle] | 868.6 | C |
| 145 | 3PP-[Orn-Hyp-cha-Trp-Nle] | 811.6 | D |
| 146 | Ac-Phe-[Orn-Pro-cha-Trp-Tyr] | 902.7 | D |
| 147 | Ac-Phe-[Orn-Pro-omf-Trp-Nle] | 860.6 | C |
| 172 | Ac-Phe-[Cys-Pro-cha-Bta-Phe-Cys]-NH₂ | 1011.6 | E |
| 173 | Ac-Phe-[Orn-Asn-cha-Trp-Nle] | 871 | E |
| 174 | Ac-Phe-[Orn-Aze-cha-Trp-Nle] | 839.5 | E |
| 175 | Ac-Phe-[Orn-Chy-cha-Trp-Nle] | 869.5 | E |
| 176 | Ac-Phe-[Orn-HyA-cha-Trp-Phe] | 945.6 | E |
| 177 | Ac-Phe-[Orn-Hyp-hle-Bta-Phe] | 894.7 | E |
| 178 | Ac-Phe-[Orn-Hyp-hle-Mcf-Phe] | 874.2 | E |
| 179 | Ac-Phe-[Orn-Hyp-hle-Pff-Nle] | 823.1 | E |
| 180 | Ac-Phe-[Orn-Hyp-hle-Pff-Phe] | 857 | E |
| 181 | Ac-Phe-[Orn-Hyp-hle-Trp-Phe] | 877.9 | D |
| 182 | Ac-Phe-[Orn-Hyp-Mmf-Trp-Nle] | 877.5 | E |
| 183 | Ac-Phe-[Orn-Hyp-Mmf-Trp-Phe] | 911.8 | E |
| 184 | Ac-Phe-[Orn-NMD-cha-Trp-Nle] | 885.5 | E |
| 185 | Ac-Phe-[Orn-Pip-hle-Bta-Phe] | 892.7 | E |
| 186 | Ac-Phe-[Orn-Pro-cha-Pff-Nle] | 833.3 | E |
| 187 | Ac-Phe-[Orn-Pro-cha-Pff-Phe] | 867.4 | E |
| 188 | Ac-Phe-[Orn-Pro-cha-Trp-1Ni] | 937.7 | E |
| 189 | Ac-Phe-[Orn-Pro-cha-Trp-Cha] | 893.6 | E |
| 190 | Ac-Phe-[Orn-Pro-cha-Trp-Chg] | 879.7 | E |
| 191 | Ac-Phe-[Orn-Pro-cha-Trp-Cit] | 897.5 | F |
| 192 | Ac-Phe-[Orn-Pro-cha-Trp-Ecr] | 891.7 | D |
| 193 | Ac-Phe-[Orn-Pro-cha-Trp-Leu] | 853.5 | E |
| 194 | Ac-Phe-[Orn-Pro-cha-Trp-nle] | 853.5 | E |
| 195 | Ac-Phe-[Orn-Pro-cha-Trp-Phe] | 887.7 | D |
| 196 | Ac-Phe-[Orn-Pro-hle-Bta-Nle] | 844.7 | E |
| 197 | Ac-Phe-[Orn-Pro-hle-Bta-Phe] | 879.5 | E |
| 198 | Ac-Phe-[Orn-Pro-hle-Pff-Phe] | 840.9 | E |
| 199 | Ac-Phe-[Orn-Pro-hle-Trp-Nle] | 828.1 | D |
| 200 | Ac-Phe-[Orn-Ser-cha-Trp-Nle] | 843.5 | E |
| 201 | Ac-Phe-[Orn-Ser-cha-Trp-Nle] | 843.5 | E |
| 202 | Ac-Phe-[Orn-Ser-hle-Trp-Nle] | 817.5 | E |
| 203 | Ac-Phe-[Orn-Thr-cha-Trp-Nle] | 858.2 | E |
| 204 | Ac-Phe-[Orn-Tic-cha-Trp-Nle] | 915.5 | E |
| 205 | Ac-Phe-[Orn-Tic-cha-Trp-Nle] | 915.5 | E |
| 311 | Ac-Thi-[Orn-Pro-hle-Bta-Phe] | 884.8 | E |
| 315 | Bzl-[Orn-Pro-cha-Bta-Nle] | 771.8 | E |
| 317 | Def-[Orn-Ser-hle-Trp-Nle] | 831.9 | E |
| 318 | Eby-Phe-[Orn-Hyp-cha-Trp-Phe] | 1008.9 | E |
| 319 | Eth-Phe-[Orn-Pro-hle-Pff-Nle] | 792.4 | E |
| 323 | Fai-Phe-[Orn-Hyp-cha-Trp-Phe] | 904.4 | E |
| 325 | Fbi-Phe-[Orn-Pro-cha-Trp-Nle] | 930.5 | E |
| 326 | Fbn-Phe-[Orn-Hyp-cha-Trp-Phe] | 966.8 | E |
| 327 | Fbn-Phe-[Orn-Pro-cha-Trp-Nle] | 916.5 | E |
| 328 | Fbn-Phe-[Orn-Pro-cha-Trp-Nle] | 916.5 | C |
| 330 | Fbo-Phe-[Orn-Pro-cha-Trp-Nle] | 924.5 | E |
| 331 | Fbp-[Orn-Pro-cha-Trp-Nle] | 839.4 | E |
| 332 | Fci-[Phe-Orn-Hyp-cha-Trp-Phe] | 973.1 | E |
| 333 | Fck-[Phe-Orn-Pro-cha-Trp-Nle] | 1046.4 | E |
| 334 | Fck-Phe-[Orn-Pro-cha-Trp-Nle] | 1047.1 | E |
| 335 | Fha-Phe-[Orn-Hyp-cha-Trp-Phe] | 988.9 | E |
| 336 | Fhb-[Phe-Orn-Hyp-cha-Trp-Phe] | 979.1 | E |
| 337 | Fhi-Phe-[Orn-Hyp-cha-Trp-Phe] | 1022 | E |
| 338 | Fhu-Phe-[Orn-Pro-hle-Pff-Nle] | 807 | E |
| 341 | H-Amf-[Orn-Aze-hle-Pff-Nle] | 750.9 | E |
| 342 | H-Bal-Phe-[Orn-Hyp-hle-Trp-Nle] | 872.5 | E |
| 343 | H-Bal-Phe-[Orn-Pro-hle-Pff-Nle] | 836 | E |
| 344 | H-Eby-[Orn-Hyp-hle-Trp-Nle] | 801.9 | E |
| 347 | Hoo-Phe-[Orn-Hyp-hle-Pff-Nle] | 921 | E |
| 353 | H-Phe-[Lys-Hyp-hle-Pff-Nle] | 795.2 | E |
| 354 | H-Phe-[Orn-Hym-hle-Mcf-Nle] | 811.4 | E |
| 355 | H-Phe-[Orn-Hym-hle-Pff-Phe] | 829.1 | E |
| 356 | H-Phe-[Orn-Hyp-cha-Trp-Nle] | 828.1 | D |
| 357 | H-Phe-[Orn-Hyp-cha-Trp-Phe] | 862.1 | D |
| 358 | H-Phe-[Orn-Hyp-ctb-Pff-Nle] | 813.2 | E |
| 359 | H-Phe-[Orn-Hyp-ctb-Trp-Nle] | 834.2 | D |
| 360 | H-Phe-[Orn-Hyp-ctb-Trp-Phe] | 868 | D |
| 361 | H-Phe-[Orn-Hyp-hle-Mcf-Leu] | 796.4 | E |
| 362 | H-Phe-[Orn-Hyp-hle-Pff-Chg] | 807 | E |
| 363 | H-Phe-[Orn-Hyp-hle-Pff-Hle] | 795.1 | E |
| 364 | H-Phe-[Orn-Hyp-hle-Pff-Leu] | 781.2 | E |
| 365 | H-Phe-[Orn-Hyp-hle-Pff-Nle] | 781.1 | E |
| 366 | H-Phe-[Orn-Hyp-hle-Pff-Phe] | 815 | E |
| 367 | H-Phe-[Orn-Hyp-hle-Trp-Hle] | 815.9 | E |
| 368 | H-Phe-[Orn-Hyp-hle-Trp-Leu] | 802.1 | D |
| 369 | H-Phe-[Orn-Hyp-hle-Trp-Nle] | 801.5 | D |
| 370 | H-Phe-[Orn-Hyp-hle-Trp-Nva] | 787.3 | E |
| 371 | H-Phe-[Orn-Hyp-hle-Trp-Phe] | 835.6 | D |
| 372 | H-Phe-[Orn-NMS-cha-Trp-Nle] | 816.1 | E |
| 373 | H-Phe-[Orn-NMS-hle-Pff-Phe] | 802.7 | E |
| 374 | H-Phe-[Orn-Pro-cha-Pff-Nle] | 790.7 | E |
| 375 | H-Phe-[Orn-Pro-cha-Pff-Phe] | 825.2 | E |
| 376 | H-Phe-[Orn-Pro-cha-Trp-Nle] | 811.5 | E |
| 377 | H-Phe-[Orn-Pro-hle-Mcf-Phe] | 815.3 | D |
| 378 | H-Phe-[Orn-Pro-hle-Ocf-Phe] | 815.3 | E |
| 379 | H-Phe-[Orn-Pro-hle-Pff-Nle] | 765.3 | E |
| 380 | H-Phe-[Orn-Pro-hle-Pff-Phe] | 799.2 | D |
| 381 | H-Phe-[Orn-Pro-hle-Trp-Nle] | 786.1 | D |
| 382 | H-Phe-[Orn-Ser-cha-Trp-Nle] | 802.1 | D |
| 383 | H-Phe-[Orn-Ser-cha-Trp-Phe] | 835.4 | D |
| 384 | H-Phe-[Orn-Ser-hle-Eaa-Nle] | 805.7 | E |
| 385 | H-Phe-[Orn-Ser-hle-Mcf-Leu] | 771.5 | E |
| 386 | H-Phe-[Orn-Ser-hle-Ocf-Nle] | 771.3 | E |
| 387 | H-Phe-[Orn-Ser-hle-Pff-Leu] | 755.2 | E |
| 388 | H-Phe-[Orn-Ser-hle-Pff-Nle] | 754.8 | D |
| 389 | H-Phe-[Orn-Ser-hle-Pff-Phe] | 788.7 | E |
| 390 | H-Phe-[Orn-Ser-hle-Trp-Nle] | 775.7 | D |
| 392 | Ohf-[Orn-Hyp-hle-Trp-Nle] | 802.4 | E |
| 393 | Tmg-Phe-[Orn-Hyp-cha-Trp-Phe] | 959.9 | E |
| 50 | Ac-Phe-Orn-Pro-cha-Trp-Arg-NH₂ | 913.3 | E |
| 51 | Ac-Phe-Orn-Pro-cha-Trp-Phe-NH₂ | 904.5 | D |
| 52 | Ac-Phe-Orn-Aze-cha-Bta-Phe-NH₂ | 907.5 | C |
| 53 | Ac-Phe-Orn-Pro-cha-Bta-2Ni—NH₂ | 954.4 | D |
| 54 | Ac-Phe-Orn-Pro-cha-Bta-Cha-NH₂ | 910.5 | E |
| 55 | Ac-Phe-Orn-Pip-cha-Trp-Phe-NH₂ | 941.3 | D |
| 60 | Ac-Phe-Lys-Ala-Cha-Ala-Leu-ala-Tyr-OH | 978.9 | F |
| 61 | Ac-Phe-Orn-Pro-cha-Trp-Phe-NH₂ | 904.9 | D |
| 62 | Ac-Phe-Orn-Pro-cha-Bta-Phe-NH₂ | 921.8 | D |
| 64 | Ac-Phe-Orn-Pro-cha-Trp-2Ni-NH₂ | 954.9 | D |
| 65 | Ac-Phe-Orn-Pro-cha-Trp-Cha-NH₂ | 911.1 | E |
| 66 | Ac-Thi-Orn-Aze-cha-Bta-Phe-NH₂ | 913.5 | C |
| 67 | Ac-Thi-Orn-Pip-cha-Bta-Phe-NH₂ | 941.3 | D |
| 68 | Ac-Phe-Orn-Pro-cha-Trp-Eap-NH₂ | 960.9 | F |
| 69 | Me2-Phe-Orn-Pro-cha-Trp-Phe-NH₂ | 890.8 | E |
| 70 | Ph2-CH—CH2—CO-Orn-Pro-cha-Trp-Phe-NH₂ | 923.7 | F |
| 71 | Ac-Ebw-Orn-Pro-cha-Trp-Phe-NH₂ | 980.8 | F |
| 72 | Ac-Phe-Orn-Pro-cha-Trp-NH—CH₂—CH₂-Ph | 861.8 | F |
| 73 | Ac-Phe-Orn-Aze-cha-Bta-NH—CH₂—CH₂-Ph | 864.7 | F |
| 74 | H-Phe-Orn-Pro-cha-Trp-Phe-NH₂ | 862.7 | E |
| 75 | H-Me-Phe-Orn-Pro-cha-Trp-Phe-NH₂ | 876.7 | E |
| 76 | Bu-NH—CO-Phe-Orn-Pro-cha-Trp-Phe-NH₂ | 961.8 | F |
| 77 | Ac-Thi-Orn-Pro-cha-Trp-Phe-NH₂ | 910.7 | E |
| 78 | Ac-Ebw-Orn-Pro-cha-Trp-Phe-NH₂ | 980.8 | E |
| 79 | Ac-Phe-Orn-Ala-cha-Trp-Phe-NH₂ | 878.7 | E |
| 80 | Ac-Phe-Orn-Pro-cha-Trp-Thi-NH₂ | 910.7 | E |
| 81 | Ac-Phe-Orn-Aze-cha-Pcf-Phe-NH₂ | 885.7 | F |
| 82 | Ac-Phe-Orn(Ac)-Pro-cha-Trp-Phe-NH₂ | 946.9 | E |
| 83 | Ac-Phe-Orn-Aze-cha-Trp-Phe-NH₂ | 890.9 | D |
| 84 | Ac-Phe-Trp-Pro-cha-Trp-Phe-NH₂ | 976.5 | E |
| 85 | Ph-NH—CO-Phe-Orn-Pro-cha-Trp-Phe-NH₂ | 981.7 | E |
| 86 | Bu-O—CO-Phe-Orn-Pro-cha-Trp-Phe-NH₂ | 963.2 | F |
| 87 | Ac-Phe-Lys-Pro-cha-Trp-Phe-NH₂ | 918.4 | E |
| 88 | Ac-Phe-Arg-Pro-cha-Trp-Phe-NH₂ | 946.4 | D |
| 89 | Ac-Phe-Gln-Pro-cha-Trp-Phe-NH₂ | 918.4 | F |
| 90 | Ac-Phe-Ser-Pro-cha-Trp-Phe-NH₂ | 877.3 | F |
| 91 | Ac-Phe-Glu-Pro-cha-Trp-Phe-NH₂ | 919.3 | F |
| 92 | Ac-Phe-Orn-Pip-cha-Trp-Phe-NH₂ | 919.8 | E |
| 93 | Ac-Phe-Orn-Hyp-cha-Trp-Phe-NH₂ | 920.3 | F |
| 94 | Ac-Phe-Orn-Pro-cha-Trp-1Ni-NH₂ | 934.5 | D |
| 95 | Ac-Phe-Orn-Aze-cha-Bta-Phe-NH—Me | 921.6 | F |
| 96 | CH3—SO2-Phe-Orn-Aze-cha-Bta-Phe-NH₂ | 943.9 | D |
| 99 | Ac-Phe-Orn-Aze-cha-Pff-Phe-NH₂ | 869.7 | E |
| 100 | Ac-Phe-Orn-Aze-cha-Mcf-Phe-NH₂ | 885.7 | E |
| 101 | Ac-Phe-Orn(Ac)-Aze-cha-Bta-Phe-NH₂ | 921.7 | D |
| 102 | Ac-Ebw-Orn-Pro-cha-Trp-Phe-NH₂ | 980.8 | E |
| 103 | Ac-Phe-Trp-Pro-cha-Trp-Phe-NH₂ | 876.5 | E |
| 104 | Ac-Phe-Arg-Pro-cha-Trp-Phe-NH₂ | 946.4 | E |
| 105 | Ac-Phe-Orn-Pip-cha-Trp-Phe-NH₂ | 919.8 | E |
| 106 | 3PP-Orn-Aze-cha-Bta-Phe-NH₂ | 850.8 | E |
| 107 | Ac-Phe-Orn-Tic-cha-Trp-Phe-NH₂ | 966.3 | E |
| 108 | Ac-Phe-Orn-Ser-cha-Trp-Phe-NH₂ | 894.5 | D |
| 109 | Ac-Phe-Orn-Pro-chg-Trp-Phe-NH₂ | 890.4 | E |
| 110 | Ac-Phe-Orn-Pro-hch-Trp-Phe-NH₂ | 918.5 | D |
| 111 | Ac-Phe-Orn-Pro-cha-Trp-Phg-NH₂ | 890.4 | F |
| 112 | Ac-Phe-Bta-Aze-cha-Bta-Phe-NH₂ | 996.6 | D |
| 113 | Ac-Phe-Trp-Pro-cha-Bta-Phe-NH₂ | 993.7 | E |
| 115 | Ac-Phe-Orn-Pip-cha-Trp-Phe-OH | 919.4 | F |
| 116 | Ac-Phe-Orn-Tic-cha-Trp-Phe-OH | 967.7 | F |
| 117 | Ac-Phe-Orn-Ser-cha-Trp-Phe-OH | 895.7 | F |
| 118 | Ac-Phe-Orn-Pro-chg-Trp-Phe-OH | 891.8 | F |
| 119 | Ac-Phe-Eec-Pro-cha-Bta-Phe-NH₂ | 1041.7 | E |
| 120 | Ac-Phe-Nle-Pro-cha-Bta-Phe-NH₂ | 920.5 | E |
| 121 | Ac-Phe-Har-Pro-cha-Bta-Phe-NH₂ | 978.0 | D |
| 122 | Ac-Phe-Arg-Pro-cha-Bta-Phe-NH₂ | 964.0 | D |
| 123 | Ac-Phe-Cys(Acm)-Pro-cha-Bta-Phe-NH₂ | 981.5 | F |
| 124 | Ac-Phe-Mpa-Pro-cha-Bta-Phe-NH₂ | 955.7 | E |
| 125 | Ac-Eby-Orn-Pro-cha-Bta-Phe-NH₂ | 921.7 | D |
| 126 | Ac-Phg-Orn-Pro-cha-Bta-Phe-NH₂ | 907.8 | E |
| 127 | Ac-Phe-Paf-Pro-cha-Bta-Phe-NH₂ | 969.6 | F |
| 128 | H2N—CO-Phe-Orn-Pro-cha-Bta-Phe-NH₂ | 922.8 | D |
| 129 | Me—O—CO-Phe-Orn-Pro-cha-Bta-Phe-NH₂ | 937.8 | E |
| 130 | (—CO—CH2—NH—CO—)-Phe-Orn-Pro-cha-Bta-Phe-NH₂ | 962.9 | E |
| 132 | Ac-Phe-Orn-Pro-hch-Trp-Phe-OH | 919.8 | E |
| 133 | (—CO—CH2—CH2—CO—)-Phe-Orn-Pro-cha-Bta-Phe-NH₂ | 961.9 | F |
| 134 | tBu-CO-Phe-Orn-Pro-cha-Bta-Phe-NH₂ | 963.9 | E |
| 135 | Ac-Lys-Phe-Orn-Aze-cha-Bta-Phe-NH₂ | 1036.0 | C |
| 136 | Ac-Gly-Phe-Orn-Aze-cha-Bta-Phe-NH₂ | 965.0 | D |
| 137 | Ac-Arg-Phe-Orn-Aze-cha-Bta-Phe-NH₂ | 1064.1 | D |
| 138 | Ac-His-Phe-Orn-Aze-cha-Bta-Phe-NH₂ | 1045.0 | E |
| 139 | Ac-Ser-Phe-Orn-Aze-cha-Bta-Phe-NH₂ | 995.0 | E |
| 140 | Ac-Guf-Phe-Orn-Aze-cha-Bta-Phe-NH₂ | 1112.1 | E |
| 141 | Ac-Dab-Phe-Orn-Aze-cha-Bta-Phe-NH₂ | 1008.0 | E |
| 142 | FH2C—CO-Phe-Orn-Pro-cha-Bta-Phe-NH₂ | 939.8 | D |
| 143 | Ac-Phe-Orn(Et2)-Pro-cha-Trp-Phe-NH₂ | 960.9 | E |
| 148 | Ac-Phe-N(nBu)-CH2—CO-Pro-cha-Trp-Phe-NH₂ | 920.8 | F |
| 149 | Ac-Phe-Orn-Pro-hle-Bta-Phe-NH₂ | 895.4 | C |
| 150 | Ac-Phe-Arg(CH2—CH2)-Pro-cha-Bta-Phe-NH₂ | 990.1 | B |
| 151 | Ac-Ala-Phe-Orn-Aze-cha-Bta-Phe-NH₂ | 978.8 | D |
| 152 | Ac-Arg-Phe-Orn-Aze-cha-Bta-Phe-NH₂ | 1063.8 | D |
| 153 | Ac-Cit-Phe-Orn-Aze-cha-Bta-Phe-NH₂ | 1064.7 | D |
| 154 | Ac-Gly-Phe-Orn-Aze-cha-Bta-Phe-NH₂ | 964.7 | C |
| 155 | Ac-Gly-Phe-Orn-Aze-chg-Bta-Phe-NH₂ | 950-3 | E |
| 156 | Ac-Gly-Phe-Orn-Aze-hch-Bta-Phe-NH₂ | 978.3 | E |
| 157 | Ac-Gly-Thi-Orn-Aze-cha-Bta-Phe-NH₂ | 971 | D |
| 158 | Ac-His-Phe-Orn-Aze-cha-Bta-Phe-NH₂ | 1044.3 | E |
| 159 | Ac-Hyp-Phe-Orn-Aze-cha-Bta-Phe-NH₂ | 1020.7 | D |
| 160 | Ac-Lys-Phe-Orn-Aze-cha-Bta-Phe-NH₂ | 1035.8 | D |
| 161 | Ac-Mff-Orn-Pro-cha-Bta-Phe-NH₂ | 939.5 | E |
| 162 | Ac-Mff-Orn-Pro-hle-Bta-Phe-NH₂ | 913.4 | E |
| 163 | Ac-Mff-Orn-Pro-hle-Mcf-Mff-NH₂ | 909.9 | E |
| 164 | Ac-Mmy-Orn-Pro-hle-Pff-Phe-NH₂ | 888 | E |
| 165 | Ac-NMF-Orn-Pro-cha-Bta-Phe-NH₂ | 935.5 | E |
| 166 | Ac-Off-Orn-Pro-cha-Bta-Phe-NH₂ | 940 | D |
| 167 | Ac-Off-Orn-Pro-hle-Bta-Phe-NH₂ | 913.4 | D |
| 168 | Ac-Orn-Phe-Orn-Aze-cha-Bta-Phe-NH₂ | 1043.8 | E |
| 169 | Ac-Pff-Orn-Pro-cha-Bta-Phe-NH₂ | 940 | D |
| 170 | Ac-Pff-Orn-Pro-hle-Bta-Phe-NH₂ | 913.4 | E |
| 171 | Ac-Pff-Orn-Pro-hle-Mcf-Pff-NH₂ | 909.6 | E |
| 206 | Ac-Phe-Ala-Pro-cha-Bta-Phe-NH₂ | 878.5 | E |
| 207 | Ac-Phe-Arg-Pro-hle-Bta-Phe-NH₂ | 937.7 | E |
| 208 | Ac-Phe-Arg-Pro-hle-Mcf-Phe-NH₂ | 915.9 | E |
| 209 | Ac-Phe-Cit-Hyp-hle-Bta-Phe-NH₂ | 954.7 | E |
| 210 | Ac-Phe-Cit-Pro-cha-Bta-Phe-NH₂ | 964.7 | E |
| 211 | Ac-Phe-Cit-Pro-hle-Bta-Phe-NH₂ | 939 | D |
| 212 | Ac-Phe-Cit-Ser-hle-Bta-Phe-NH₂ | 928.7 | E |
| 213 | Ac-Phe-Dab-Aze-cha-Bta-Phe-NH₂ | 894 | D |
| 214 | Ac-Phe-Dab-Aze-hle-Bta-Phe-NH₂ | 868.1 | D |
| 215 | Ac-Phe-Dab-Pro-cha-Bta-Phe-NH₂ | 907.9 | C |
| 216 | Ac-Phe-Dap-Pro-cha-Bta-Phe-NH₂ | 893.7 | E |
| 217 | Ac-Phe-Ech-Pro-cha-Bta-Phe-NH₂ | 1033.7 | E |
| 218 | Ac-Phe-Eep-Pro-cha-Bta-Phe-NH₂ | 1013.5 | E |
| 219 | Ac-Phe-Fcn-Aze-cha-Bta-Phe-NH₂ | 961.9 | C |
| 220 | Ac-Phe-Fcn-Pro-cha-Bta-Phe-NH₂ | 975.9 | C |
| 221 | Ac-Phe-Fco-Pro-cha-Bta-Phe-NH₂ | 935.8 | D |
| 222 | Ac-Phe-Fco-Pro-cha-Bta-Phe-NH₂ | 962 | E |
| 223 | Ac-Phe-Fcp-Aze-cha-Bta-Phe-NH₂ | 1444 | D |
| 224 | Ac-Phe-Ffa-Aze-cha-Bta-Phe-NH₂ | 976 | D |
| 225 | Ac-Phe-Ffa-Pro-cha-Bta-Phe-NH₂ | 990 | D |
| 226 | Ac-Phe-Ffa-Pro-hle-Bta-Phe-NH₂ | 964 | C |
| 227 | Ac-Phe-G23-Pro-cha-Bta-Phe-NH₂ | 1000.3 | E |
| 228 | Ac-Phe-Guf-Pro-cha-Bta-Phe-NH₂ | 1011.9 | D |
| 229 | Ac-Phe-Har-Aze-cha-Bta-Phe-NH₂ | 964.1 | C |
| 230 | Ac-Phe-His-Pro-cha-Bta-Phe-NH₂ | 944.3 | E |
| 231 | Ac-Phe-L22-Pro-cha-Bta-Phe-NH₂ | 949.8 | C |
| 232 | Ac-Phe-OrA-Pro-cha-Bta-Phe-NH₂ | 963.6 | E |
| 233 | Ac-Phe-OrE-Pro-cha-Bta-Phe-NH₂ | 977.8 | E |
| 234 | Ac-Phe-Orn-Aze-hle-Bta-Phe-NH₂ | 881.9 | D |
| 235 | Ac-Phe-Orn-Chy-cha-Bta-Phe-NH₂ | 937.4 | E |
| 236 | Ac-Phe-Orn-Chy-hle-Pff-Phe-NH₂ | 873.8 | E |
| 237 | Ac-Phe-Orn-G24-cha-Bta-Phe-NH₂ | 923.8 | E |
| 238 | Ac-Phe-Orn-G25-cha-Bta-Phe-NH₂ | 939.8 | E |
| 239 | Ac-Phe-Orn-G26-cha-Bta-Phe-NH₂ | 961.8 | E |
| 240 | Ac-Phe-Orn-G27-cha-Bta-Phe-NH₂ | 972.7 | E |
| 241 | Ac-Phe-Orn-G30-cha-Bta-Phe-NH₂ | 1006.8 | E |
| 242 | Ac-Phe-Orn-G31-cha-Bta-Phe-NH₂ | 1045.9 | E |
| 243 | Ac-Phe-Orn-Hse-cha-Bta-Phe-NH₂ | 925.9 | E |
| 244 | Ac-Phe-Orn-Hyp-hle-Bta-Phe-NH₂ | 911.7 | E |
| 245 | Ac-Phe-Orn-Hyp-hle-Pff-Phe-NH₂ | 874 | E |
| 246 | Ac-Phe-Orn-NMA-cha-Bta-Phe-NH₂ | 909.8 | E |
| 247 | Ac-Phe-Orn-NMS-cha-Bta-Phe-NH₂ | 925.8 | E |
| 248 | Ac-Phe-Orn-Pro-cha-1Ni-Phe-NH₂ | 916 | E |
| 249 | Ac-Phe-Orn-Pro-cha-Bta-1Ni—NH₂ | 971.9 | E |
| 250 | Ac-Phe-Orn-Pro-cha-Bta-Bhf-NH₂ | 935.9 | D |
| 251 | Ac-Phe-Orn-Pro-cha-Bta-Dff-NH₂ | 957.7 | D |
| 252 | Ac-Phe-Orn-Pro-cha-Bta-Eaa-NH₂ | 933.9 | E |
| 253 | Ac-Phe-Orn-Pro-cha-Bta-L19 | 979.1 | E |
| 254 | Ac-Phe-Orn-Pro-cha-Bta-Mcf-NH₂ | 955.9 | E |
| 255 | Ac-Phe-Orn-Pro-cha-Bta-Mff-NH₂ | 939.8 | C |
| 256 | Ac-Phe-Orn-Pro-cha-Bta-NH—CH(CH₂OH)—CH₂-Ph | 964.6 | E |
| 257 | Ac-Phe-Orn-Pro-Cha-Bta-NH-NBn-CO—NH₂ | 922.8 | E |
| 258 | Ac-Phe-Orn-Pro-cha-Bta-Opa-NH₂ | 922.9 | E |
| 259 | Ac-Phe-Orn-Pro-cha-Bta-Pcf-NH₂ | 956.1 | D |
| 260 | Ac-Phe-Orn-Pro-cha-Bta-Pmf-NH₂ | 935.8 | D |
| 261 | Ac-Phe-Orn-Pro-cha-Bta-Thi-NH₂ | 927.8 | C |
| 262 | Ac-Phe-Orn-Pro-cha-Otf-Phe-NH₂ | 933.9 | E |
| 263 | Ac-Phe-Orn-Pro-ctb-Bta-Phe-NH₂ | 927.4 | D |
| 264 | Ac-Phe-Orn-Pro-ctb-Eaa-Phe-NH₂ | 940.2 | D |
| 265 | Ac-Phe-Orn-Pro-ctb-Mcf-Phe-NH₂ | 906.3 | E |
| 266 | Ac-Phe-Orn-Pro-ctb-Pff-Phe-NH₂ | 890.1 | D |
| 267 | Ac-Phe-Orn-Pro-hch-Trp-Phe-OH | 919.8 | E |
| 268 | Ac-Phe-Orn-Pro-hle-1Ni-Phe-NH₂ | 889.7 | D |
| 269 | Ac-Phe-Orn-Pro-hle-6FW-Phe-NH₂ | 897 | E |
| 270 | Ac-Phe-Orn-Pro-hle-Bta-1Ni—NH₂ | 945.8 | E |
| 271 | Ac-Phe-Orn-Pro-hle-Bta-2Ni—NH₂ | 946 | E |
| 272 | Ac-Phe-Orn-Pro-hle-Bta-5Ff-NH₂ | 985.7 | E |
| 273 | Ac-Phe-Orn-Pro-hle-Bta-Aic-NH₂ | 908 | E |
| 274 | Ac-Phe-Orn-Pro-hle-Bta-Cha-NH₂ | 902 | E |
| 275 | Ac-Phe-Orn-Pro-hle-Bta-Chg-NH₂ | 888 | E |
| 276 | Ac-Phe-Orn-Pro-hle-Bta-Eaa-NH₂ | 964.4 | E |
| 277 | Ac-Phe-Orn-Pro-hle-Bta-Egy-NH₂ | 964.4 | E |
| 278 | Ac-Phe-Orn-Pro-hle-Bta-Pcf-NH₂ | 930.2 | E |
| 279 | Ac-Phe-Orn-Pro-hle-Bta-Pff-NH₂ | 913.7 | E |
| 280 | Ac-Phe-Orn-Pro-hle-Bta-Phe-NH₂ | 895.8 | D |
| 281 | Ac-Phe-Orn-Pro-hle-Bta-phe-OH | 897 | E |
| 282 | Ac-Phe-Orn-Pro-hle-Bta-Tyr-NH₂ | 911.5 | E |
| 283 | Ac-Phe-Orn-Pro-hle-Dff-Phe-NH₂ | 875.4 | E |
| 284 | Ac-Phe-Orn-Pro-hle-Eaa-Phe-NH₂ | 907.4 | E |
| 285 | Ac-Phe-Orn-Pro-hle-Egc-Phe-NH₂ | 892.8 | E |
| 286 | Ac-Phe-Orn-Pro-hle-Egy-Phe-NH₂ | 908.3 | E |
| 287 | Ac-Phe-Orn-Pro-hle-Egz-Phe-NH₂ | 885 | E |
| 288 | Ac-Phe-Orn-Pro-hle-Mcf-2Ni—NH₂ | 924.3 | E |
| 289 | Ac-Phe-Orn-Pro-hle-Mcf-Cha-NH₂ | 880.3 | D |
| 290 | Ac-Phe-Orn-Pro-hle-Mcf-Pff-NH₂ | 892.1 | E |
| 291 | Ac-Phe-Orn-Pro-hle-Mcf-Phe-NH₂ | 874.2 | E |
| 292 | Ac-Phe-Orn-Pro-hle-Mff-Phe-NH₂ | 857.9 | E |
| 293 | Ac-Phe-Orn-Pro-hle-Mmy-Phe-NH₂ | 870.1 | E |
| 294 | Ac-Phe-Orn-Pro-hle-Ocf-Phe-NH₂ | 874.1 | E |
| 295 | Ac-Phe-Orn-Pro-hle-Off-Phe-NH₂ | 857.9 | E |
| 296 | Ac-Phe-Orn-Pro-hle-Otf-Phe-NH₂ | 907.8 | E |
| 297 | Ac-Phe-Orn-Pro-hle-Pff-2Ni—NH₂ | 908.1 | E |
| 298 | Ac-Phe-Orn-Pro-hle-Pff-Cha-NH₂ | 864 | E |
| 299 | Ac-Phe-Orn-Pro-hle-Pff-Eaa-NH₂ | 926.3 | E |
| 300 | Ac-Phe-Orn-Pro-hle-Pff-Mmy-NH₂ | 888.1 | E |
| 301 | Ac-Phe-Orn-Pro-hle-Pff-Pff-NH₂ | 876 | E |
| 302 | Ac-Phe-Orn-Pro-hle-Pff-Phe-NH₂ | 857.7 | E |
| 304 | Ac-Phe-Orn-Pro-hle-Phe-Phe-NH₂ | 839.7 | E |
| 305 | Ac-Phe-Orn-Pro-hle-Tff-Phe-NH₂ | 893.8 | E |
| 306 | Ac-Phe-Orn-Pro-hle-Trp-Phe-NH₂ | 878.9 | E |
| 307 | Ac-Phe-Orn-Pro-ile-Trp-Phe-NH₂ | 864.5 | B |
| 308 | Ac-Phe-Orn-Pro-omf-Bta-Phe-NH₂ | 929.8 | E |
| 309 | Ac-Phe-Orn-Ser-cha-Bta-Phe-NH₂ | 912 | D |
| 310 | Ac-Ser-Phe-Orn-Aze-cha-Bta-Phe-NH₂ | 994.7 | C |
| 312 | Ac-Thi-Orn-Pro-cha-Bta-Phe-NH₂ | 927.8 | D |
| 313 | Ac-Thi-Orn-Pro-cha-Bta-Thi-NH₂ | 933.8 | D |
| 314 | Ac-Thr-Phe-Orn-Aze-cha-Bta-Phe-NH₂ | 1008.7 | D |
| 316 | CH3CH2CO-Phe-Orn-Pro-cha-Bta-Phe-NH₂ | 935.9 | D |
| 320 | FAc-Phe-Fib-Aze-cha-Bta-Phe-NH₂ | 1023.9 | E |
| 321 | FAc-Phe-Orn-Aze-cha-Bta-Phe-NH₂ | 925.7 | D |
| 322 | FAc-Phe-Orn-Pro-cha-Bta-Phe-NH₂ | 939.8 | D |
| 324 | Faz-Orn-Pro-cha-Bta-Phe-NH₂ | 864.7 | E |
| 329 | Fbn-Phe-Cit-Pro-hle-Bta-Phe-NH₂ | 1001.9 | E |
| 339 | Fhu-Phe-Orn-Pro-cha-Bta-Phe-NH₂ | 921.8 | E |
| 340 | Fid-Phe-Orn-Pro-cha-Bta-Phe-NH₂ | 966.6 | E |
| 345 | H-Gly-Phe-Orn-Pro-cha-Bta-Phe-NH₂ | 936.7 | E |
| 346 | H-Nip-Phe-Cit-Pro-hle-Bta-Phe-NH₂ | 1007.7 | E |
| 348 | Hoo-Phe-Cit-Pro-hle-Pff-Phe-NH₂ | 999 | E |
| 349 | Hoo-Phe-Orn-Hyp-hle-Pff-Phe-NH₂ | 971.8 | E |
| 350 | Hoo-Phe-Orn-Pro-hle-Bta-Phe-NH₂ | 994.2 | D |
| 351 | Hoo-Phe-Orn-Pro-hle-Mcf-Phe-NH₂ | 972.3 | D |
| 352 | Hoo-Phe-Orn-Pro-hle-Pff-Phe-NH₂ | 956 | D |
| 391 | H-Phe-Cit-Pro-hle-Bta-Phe-NH₂ | 896.7 | E |

### Example 12

### Determination of EC₅₀-Values in an Enzyme Release Assay

The determination of EC₅₀-values was performed according to the procedure given in example II, with the exception that 30 μl of the test compound need to be mixed with 75 μl of the cell suspension mentioned in example 11. Neither preincubation nor addition of C5a for stimulation of enzyme release is needed. The results for the compounds tested are shown in table 5.

TABLE 5
Data for agonistic activity of selected compounds according to the
present invention

| | | EC₅₀ |
|---|---|---|
| No. | Compound | (nM) |
| — | hrC5a | 2.4 |
| 3 | HOCH₂(CHOH)₄—C═N—O—CH₂—CO-Phe[OP- | >>1430 |
| | dCha-W-Nle] | |
| 41 | Ph-CH₂—CH₂—CO-[Orn-Pro-cha-Trp-Nle] | >>1430 |
| 2 | Ac-Phe-[Orn-Hyp-cha-Trp-Phe] | >>1430 |
| 42 | H-Phe-[Orn-Pro-cha-Trp-Nle] | >>1430 |
| 1 | Ac-Phe-[Orn-Pro-cha-Trp-Phe] | >>1430 |
| 43 | Ac-Lys-Phe-[OP-dCha-W-Nle] | >>1430 |
| 28 | H-Phe-[Orn-Pro-cha-Trp-Nle] | >>1430 |
| 44 | H-Phe-[Orn-Ser-cha-Trp-Nle] | >>1430 |
| 33 | Ac-Phe-[Orn-Pro-cha-Trp-Eaf] | >>1430 |
| 61 | Ac-Phe-Orn-Pro-cha-Trp-Phe-NH₂ | >100000 |
| 62 | Ac-Phe-Orn-Pro-cha-Bta-Phe-NH₂ | >100000 |
| 71 | Ac-Ebw-Orn-Pro-cha-Trp-Phe-NH₂ | >100000 |
| 88 | Ac-Phe-Arg-Pro-cha-Trp-Phe-NH₂ | >100000 |
| 55 | Ac-Phe-Orn-Pip-cha-Trp-Phe-NH₂ | >100000 |
| 83 | Ac-Phe-Orn-Aze-cha-Trp-Phe-NH₂ | >100000 |
| 84 | Ac-Phe-Trp-Pro-cha-Trp-Phe-NH₂ | >100000 |
| 67 | Ac-Thi-Orn-Pip-cha-Bta-Phe-NH₂ | >100000 |

### Example 13

### Solubility Determination for Selected C5aR-Antagonists

The solubility of compounds was determined by the following procedure: 20 μl of a 10 mM stock solution in DMSO were diluted in 980 μl of a buffer system of choice. After incubation for 24 h at RT in a shaker and centrifugation at 11.000 rpm the supernatant was removed and the UV-absorption of the supernatant was measured and related to the absorption of a control sample solved in 60% MeOH which served as reference for the calculation of the solubility. Compounds that showed similar solubility in a buffer system of choice and in the control system were subsequently tested for their maximum solubility. Therefor the compound was suspended at 10 mg/ml in a solvent system of choice. The undissolved part was removed by centrifugation after 24 h. The UV-absorption of the supernatant was measured and compared to a reference solution of the compound in 60% MeOH. The solubility of some compounds according to the invention is shown in table 6.

TABLE 6
Solubility of some selected peptides

| | | Solubility in 20 mM HEPES |
|---|---|---|
| No. | Compound | pH 7.4 (% of 200 μM) |
| 1 | Ac-Phe-[Orn-Pro-cha-Trp-Phe] | 8 |
| 2 | Ac-Phe-[Orn-Hyp-cha-Trp-Phe] | 13 |
| 28 | Ac-Phe-[Orn-Pro-cha-Trp-Nle] | 22 |
| 42 | H-Phe-[Orn-Pro-cha-Trp-Phe] | 45 |
| 4 | X-Phe-[Orn-Pro-cha-Trp-Nle]; | 84 |
| | X = 2-acetamido-1-methyl- | |
| | glucuronyl | |
| 40 | Ac-Phe-[Orn-Pro-cha-Trp-Arg] | 94 |
| 43 | Ac-Lys-Phe-[Orn-Pro-cha-Trp-Nle] | 93 |

### Example 14

### Development of a Pharmacophor Model Based on Antagonists

The exchange of arginine in compound 40 by alanine (39) outlines the importance of the side-chain in this position for the inhibitory activity of the peptide. The replacement of arginine by the positively charged amino acid lysine (22) surprisingly results in an increase of the IC₅₀ value (from 20 nM to 8700 nM). This means that the positive charge alone is not responsible for the antagonistic activity. The introduction of 4-aminophenylalanine (Paf) to the C-terminal position (14) results in an IC₅₀-value of 30 nM. Interestingly, the amino-group in Paf has a similar distance to the Cα-atom compared to the amino group in Lys. The exchange of arginine in compound 40 with the uncharged very hydrophobic phenylalanine results in compound 1, which surprisingly shows a similar IC₅₀-value (23 nM) to compound 40. This clearly shows that it is not the positive charge of the side chain of Arg or Paf which is responsible for the important interaction with C5aR, but the hydrophobic part of Paf, Phe or the aliphatic side chain in Arg. It is possible to replace the arginine by other, hydrophobic moieties, without significant loss of activity compared to 40. Examples for these types of substitutions are shown in compounds 1, 28, 29, 31, 32, 33, 34, 35, 36, 37, 38.

The exchange of other amino acids in 40 by Ala, N-Me-Ala, or D-Ala revealed that the side chains of the following amino acids are important for antagonistic activity: Phe, cha, Trp.

A pharmacophor model was developed based on the structure-activity relationship of these and additional peptides. The distances for the important residues (two hydrophobic and two aromatic groups) for activity are proposed by the following method:

The pharmacophor model was developed based on a molecular dynamic simulation (duration 2 ns with an increment of 2 fs) of compound 28. The simulation was performed using AMBER94-force field and a water-model (TIP3) under periodic frame work. The static analysis of the snapshots from the last nanosecond of the trajectory (1000 structures) gave the distances between the mass-centered pharmacophor groups (see below).

The starting structure for the molecular dynamic simulation was based on ensemble-dynamic calculations with seven cyclic peptides. The peptides were highly active (IC₅₀ in the lower nanomolar range) and with structure-restricting properties when compared to each other.

### Example 15

### Measurement of the AB-Permeability in a TC-7 Based Assay-System

The screening compounds are diluted to a 50 μM solution in HBSS-MES (5 mM, pH 6.5) from 10 mM stock solution in 100% DMSO. ¹⁴C-mannitol (approx. 4 μM) is added to the sample. The solution is centrifuged and the supernatant is added to the apical side of the TC-7 cell culture (passage 15, in 24 well transwell plate) to a final DMSO-concentration of 1%. HBSS-HEPES (5 mM, pH 7.4) is placed at the basolateral side. The cells were incubated for 120 min at 37° C. The integrity of the TC-7 cell-layer was tested with mannitol to show a permeability (Pₐₚₚ)<2.5 10⁻⁶ cm/s. The permeability Pₐₚₚ [cm/s] is derived from the equation (V_{R}xC_{R120})/(ΔtxAx(C_{D,mid}-C_{R,mid})) whereas V_{R} is the volume of the receiver chamber, C_{R120} the concentration of the test article in the receiver camber after 120 min, Δt the incubation time, A the area of the TC-7 cell-layer, C_{D,mid} the midpoint concentration of the test article in the donor chamber and C_{R,mid} the concentration of the test article in the receiver chamber.

| Compound | AB-permeability [cm/s] |
|---|---|
| Ac-Phe[Orn-Pro-cha-Trp-Arg] | 0.52 |
| Ac-Phe[Orn-Hyp-cha-Trp-Phe] | 14.25 |

### Example 16

### Synthesis of Ac-Phe-Orn-Pro-cha-Trp-Phe-NH₂ (51)

The peptide was prepared according to AAV1. Purification by reversed phase HPLC yielded 10.0 mg of 51 as a white solid.

MS (ESI): m/z=904.5 [(M+H)⁺].

### Example 17

### Synthesis of Ac-Phe-Orn-Aze-cha-Bta-Phe-NH₂ (52)

The peptide was prepared according to AAV1. Purification by reversed phase HPLC yielded 10.5 mg of 52 as a white solid.

MS (ESI): m/z=907.5 [(M+H)⁺].

### Example 18

### Synthesis of Ac-Phe-Orn-Pro-cha-Trp-NH—CH₂—CH₂-Ph (72)

200 mg bromo-(4-methoxyphenyl)methyl polystyrene resin was incubated with 5 ml of a 50% solution of phenylethylamine in THF (v/v) at RT for 18 h. The resin was washed (DMF; 3×5.0 ml, MeOH; 3×5.0 ml, DCM; 3×5.0 ml) and the peptide was prepared according to AAV1. Purification by reversed phase HPLC yielded 4.1 mg of 72 as a white solid.

MS (ESI): m/z=861.8 [(M+H)⁺].

### Example 19

### Synthesis of Ac-Phe-Orn-Aze-cha-Bta-Phe-NH-Me (95)

4.5 g 4-(4-formyl-3-methoxy-phenoxy)-butyl-acid-polystyrene resin was swollen for 15 min in THF. The resin was filtered off and a mixture of 3.04 g (10 eq.) methylamine-hydrochloride, 2.7 ml acetic acid, 2.7 ml trimethylorthoformiate and 90 ml THF was added and gently stirred. After one hour 2.83 g (10 eq.) sodium cyanoborhydride in 45 ml DMF was added. The mixture was stirred over night, the resin was filtered off and washed with DMF (5×), MeOH (5×) und CH₂Cl₂ (5×). Amino acid coupling was performed using 968 mg (5 eq.) Fmoc-Phe-OH, 950 mg (5 eq.) HATU and 3.75 ml DIPEA in 10 ml DMF for two hours. The resin was filtered off and washed with DMF (5×), MeOH (5×) und CH₂Cl₂ (5×). 200 mg of the obtained resin was further used. The peptide was prepared according to AAV1. Purification by reversed phase HPLC yielded 10.0 mg of 95 as a white solid.

MS (ESI): m/z=921.6 [(M+H)⁺].

### Example 20

### Synthesis of CH₃—SO₂-Phe-Orn-Aze-cha-Bta-Phe-NH₂ (96)

The peptide was prepared according to AAV1. CH₃—SO₂—Cl was used as N-terminal “amino acid”. Purification by reversed phase HPLC yielded 5.5 mg of 96 as a white solid.

MS (ESI): m/z=943.9 [(M+H)⁺].

### Example 21

### Synthesis of H₂N—CO-Phe-Orn-Pro-cha-Bta-Phe-NH₂ (128)

The resin-bound peptide H-Phe-Orn-Pro-cha-Bta-Phe-Rink-amide resin was prepared according to AAV1. Diphenylmethylisocyanate (5 eq.) and DIPEA (10 eq.) in DMF were reacted for two hours with the N-terminal amino group. Cleavage from the resin with 95% TFA, 2.5% water and 2.5% TIPS and subsequent purification by reversed phase HPLC yielded 0.92 mg of 128 as a white solid.

MS (ESI): m/z=922.8 [(M+H)⁺].

### Example 22

### Synthesis of (—CO—C1H₂—NH—CO—)-Phe-Orn-Pro-cha-Bta-Phe-NH₂ (130)

The resin-bound peptide H-Gly-Phe-Om-Pro-cha-Bta-Phe-Rink-amide resin was synthesized according to AAV1. The peptide was incubated for three hours with disuccinimidylcarbonate (3 eq.) and DIPEA (3 eq.) in DMF. Afterwards additional 3 eq. DIPEA were added and the reaction was continued for five hours at RT. Cleavage from the resin with 95% TFA, 2.5% water, and 2.5% TIPS and subsequent purification by reversed phase HPLC yielded 3.8 mg of 130 as a white solid.

MS (ESI): m/z=962.9 [(M+H)⁺].

### Example 23

### Synthesis of (—CO—CH₂—CH₂—CO—)-Phe-Orn-Pro-cha-Bta-Phe-NH₂ (133)

The resin-bound peptide H-Phe-Orn-Pro-cha-Bta-Phe-Rink-amide resin was synthesized according to AAV1. Afterwards succinic anhydride (5 eq.) and DIPEA (10 eq.) in DMF were incubated with the N-terminal amino group for two hours. The resin was filtered off and washed with DMF (5×), MeOH (5×), and CH₂Cl₂ (5×). Finally the resin was incubated with HBTU (5 eq.) and DIPEA (10 eq.) in DMF for one day. Cleavage from the resin with 95% TFA, 2.5% water and 2.5% TIPS and subsequent purification by reversed phase HPLC yielded 0.47 mg of 133 as a white solid.

MS (ESI): m/z=961.9 [(M+H)⁺].

### Example 24

### Synthesis of FH₂C—CO-Phe-Orn-Pro-cha-Bta-Phe-NH₂ (142)

The peptide was prepared according to AAV1, whereas fluoro-acetic acid was employed as N-terminal amino acid. Purification by reversed phase HPLC yielded 0.9 mg of 142 as a white solid.

MS (ESI): m/z=939.8 [(M+H)⁺].

### Example 25

### Synthesis of Ac-Phe-Orn(Et₂)-Pro-cha-Trp-Phe-NH₂ (143)

The peptide was prepared according to AAV1. Purification by reversed phase HPLC yielded 10.0 mg of 51 as a white solid. 5.0 mg of the peptide was dissolved in THF and 1 ml acetaldehyde was added. The suspension was stirred for 12 h at RT after addition of 100 mg (polystyrene methyl)trimethyl-ammoniumcyanoborhydride (3 mmol/g). The resin was filtered off and the mixture was evaporated to dryness. Purification by reversed phase HPLC yielded 1.2 mg of 143.

MS (ESI): m/z=960.9 [(M+H)⁺].

### Example 26

### Synthesis of Ac-Phe-N(¹¹Bu)-CH₂—CO-Pro-cha-Trp-Phe-NH₂ (144)

The resin bound peptide H-Pro-cha-Trp-Phe-Rink-amide resin was performed according to AAV1. The free amino group was incubated with 4 ml of a 0.4 M solution of bromoactic acid anhydride in DCM (2×15 min). The resin was washed (DMF; 3×5.0 ml, MeOH; 3×5.0 ml, DCM; 3×5.0 ml) and then incubated for 2×30 min in 4 ml of a 5 M solution of n-butylamine. After washing the resin (DMF; 3×5.0 ml, MeOH; 3×5.0 ml, DCM; 3×5.0 ml) the synthesis of the remaining peptide was performed according to AAV1.

### Example 27

### Synthesis of Ac-Phe-Arg(CH₂CH₂)-Pro-cha-Bta-Phe-NH₂ (150)

Peptide synthesis according to AAV1 yielded 700 mg of Ac-Phe-Orn-Pro-cha-Bta-Phe-NH₂ (62) as crude product. 15 mg of 62 (0.016 mmol) were incubated with 39.7 mg (10 eq.) 2-methylthio-2-imidazoline-hydroiodine and 55.4 μl (20 eq.) DIPEA in 1 ml MeCN at 40° C. for 24 h. The solvent was removed under vacuum and the product was purified by HPLC. Freeze drying with 1 ml 0.1 N HCl and 0.5 ml MeCN yielded 0.7 mg of 150 as white solid.

MS (ESI): m/z=960.9 [(M+H)⁺].

### Example 28

### Efficacy of compound 149 in a model of immune complex mediated peritonitis

Immune complex mediated peritonitis resembles the pathological conditions of immune complex related diseases like vasculitis, nephritis, arthritis, and farmer's disease. The associated animal model was described by Heller et al. (1999 Journal of Immunology 163: 985-994) and takes advantage of the pro-inflammatory effects of immune complexes formed after i.v. administration of the antigen and i.p. administration of the antibody.

BALB/c mice (6-8 weeks old) were treated i.v. with compound 149 (1 mg/kg bodyweight in 200 μl vehicle) 15 min before the initiation of the reverse passive Arthus reaction. Arthus reaction was induced through the administration of OVA (20 mg/kg i.v. in 200 μl PBS) and polyclonal anti-OVA Ab (rabbit; 800 μg/Maus i.p). After 6 h a peritoenal lavage with 2 ml PBS 0.1% BSA was done. The collected PE-cells were stained with DIFF-Quick. At least 20 visual fields (100× magnification) were analyzed for the presence of neutrophils.

FIG. 1 shows the reduction of the influx of pro-inflammatory cells into the peritoneum caused by treatment with 149.

### Example 29

### Efficacy of compound 149 in a model of C5a induced neutropenia

C5a induced neutropenia is a model for shock induced diseases (e.g. septic shock), where the systemic role of C5a (neutropenia, blood pressure decrease) plays an important role. The reason for the decrease of the neutrophils in the circulation is their binding to the vessel walls due to the C5a stimulus. These processes of neutrophil recruitment are playing an important role in many other diseases like reperfusion injury. This model was also described by Short et al. (1999 British Journal of Pharmacology 125: 551-554).

Female Wistar rats were treated i.p. with ketamine (80 mg/kg) and xylazine (12 mg/kg). A catheter was introduced in the jugular vein and the animals were subjected to the following procedure:

1. Rats were treated with vehicle or compounds according to the invention like compound 149 via i.v. infusion. A blood sample was taken one minute before compound treatment.
2. 10 min after compound infusion rats were treated with 2 μg/kg hr C5a i.v. (2 μg/kg over 1 min).

Blood samples were taken shortly before and after C5a administration.

3. Blood samples (approx. 0.2 ml) in lithium-heparin vials from the jugular vein were used for the differential blood count.

### White Blood Cell Count:

White blood cell count was measured with a haematology-cell-counter.

### Differential Cell Count:

Blood smears were prepared out of the heparinized blood samples. Each sample was dehydrated prior to staining with methanol. After fixation the samples were stained with May Grünwald staining for 5 min. This was followed by a washing step with aqua dest. Subsequently, a Giemsa staining was done for 2 min and the samples were washed again.

The differential cell count was determined as the sum of neutrophils, eosinophils, basophils, lymphocytes and monocytes of 100 cells. Then the percentage of the neutrophils in relation to all white blood cells was calculated.

The result is presented in FIG. 2 and shows clearly, that the administration of compound 149 reduces the C5a-induced neutropenia remarkable. Therefore the intended anti-inflammatory effect is realized in this inflammatory model.

### Example 30

### Comparison of activity of peptides with different C-terminal amino acids

The assay system described in example 11 was used to measure the activity of compounds 10 and 40:

| | | | |
|---|---|---|---|
| 10 | Ac-Phe-[Orn-Pro-cha-Trp-Cit] | 897.5 | F |
| 40 | Ac-Phe-[Orn-Pro-cha-Trp-Arg] | 896.6 | C |

Note the drop in activity when the charged arginine (activity class C; <=20 nM) is replaced by the uncharged citrulline (activity class F; >200 nM).

The guanidine-group in Arg and the urea-group in Cit are bioisosteres with similar space filling properties. This fact points out how important the positive charge is, which is already described in international patent application WO 03/033528. Furthermore this example demonstrates that the size of a residue alone is not an appropriate criterion for predicting activity.

Another aspect is the fact that citrulline is uncharged under physiological conditions, but quite polar, even so not as polar as the charged guanidine. This becomes clear, when the logP-values of different amino acids are calculated, as shown below:

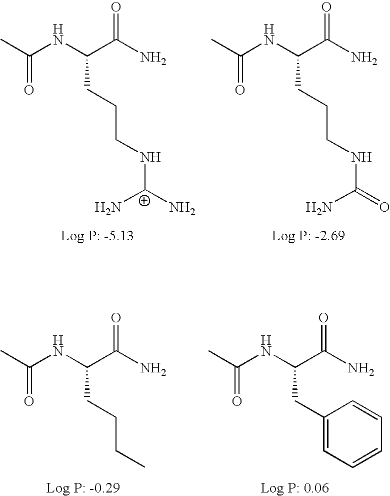

The logP-value reflects the distribution coefficient of a compound between n-octanol and water. More polar compounds show a lower logP-value. All logP-values shown above were calculated with the program Chemdraw (Cambridge Soft, Cambridge, UK).

Due to the huge loss of activity when the polar guanidine-group is replaced by the medium polar urea group the one skilled in the art would not use even more unpolar groups at this position, as much less remaining activity would be expected.

The features of the invention disclosed in the above description, the claims or the drawings can individually or in any combination be essential to the practice of the invention in its various embodiments.

1. A compound, preferably a C5a receptor antagonist, having the following structure: 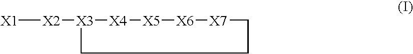
, whereby
X1 is a radical having a mass of about 1-300, whereby X1 is preferably selected from the group comprising R5-, R5-CO—, R5-N(R6)-CO—, R5-O—CO—, R5-SO₂—, R5-N(R6)-SO₂—, R5-N(R6)-, R5-N(R6)-CS—, R5-N(R6)-C(NH)—, R5-CS—, R5-P(O)OH—, R5-B(OH)—, R5-CH═N—O—CH₂—CO—, in which R5 and R6 are individually and independently selected from the group comprising H, F, hydroxy, alkyl, substituted alkyl, cycloalkyl, substituted cycloalkyl, heterocyclyl, substituted heterocyclyl, arylalkyl, substituted arylalkyl, aryl, substituted aryl, heteroaryl, substituted heteroaryl, acyl, substituted acyl, alkoxy, alkoxyalkyl, substituted alkoxyalkyl, aryloxyalkyl and substituted aryloxyalkyl,
X2 is a radical that mimics the biologic binding characteristics of a phenylalanine unit,
X3 and X4 individually and independently are a spacer, whereby the spacer is preferably selected from the group comprising amino acids, amino acid analogs and amino acid derivates,
X5 is a radical that mimics the biologic binding characteristics of a cyclohexylalanine or homoleucine unit,
X6 is a radical that mimics the biologic binding characteristics of a tryptophane unit,
X7 is a radical that mimics the biologic binding characteristics of a norleucine or phenylalanine unit,
a chemical bond between X3 and X7 is formed, and
the lines—in formula (I) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

2. The compound according to claim 1, characterized by X3 and X7 each being an amino acid, amino acid analog or amino acid derivative, whereby the chemical bond between X3 and X7 is formed under participation of at least one moiety of X3 and X7, and the moieties for X3 and X7 are individually and independently selected from the group comprising the C terminus, the N terminus and the respective side chain of the amino acid.

3. The compound according to claim 1, whereby
X1 is a radical having a mass of about 1-300, whereby X1 is preferably selected from the group comprising R5, R5-CO—, R5-N(R6)-CO—, R5-O—CO—, R5-SO₂—, R5-N(R6)-C(NH)—, whereby R5 and R6 are individually and independently selected from the group comprising H, alkyl, substituted alkyl, cycloalkyl, substituted cycloalkyl, heterocyclyl, substituted heterocyclyl, aryl and substituted aryl;
X2 and X6 are individually and independently an aromatic amino acid, a derivative or an analog thereof,
X5 and X7 are individually and independently a hydrophobic amino acid, a derivative or an analog thereof.

4. The compound according to claim 1, whereby X2, X5, X6 and X7 individually and independently have the following structure: 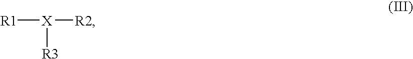
whereby
X is C(R4) or N,
R1 is optionally present and if R1 is present then R1 is a radical that is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, cycloalkylalkyl and substituted cycloalkylalkyl;
R2 is optionally present and if R2 is present then R2 is a radical, that is selected from the group comprising >C═O, >C═S, >SO₂, >S═O, >C═NH, >C═N—CN, >PO(OH), >B(OH), >CH₂, >CH₂CO, >CHF and >CF₂;
R4 is a radical, whereby the radical is selected from the group comprising H, F, CH₃, CF₃, alkyl and substituted alkyl;
the binding of structure (III) to the moieties of the molecule X1 and X3, X4 and X6, X5 and X7, and X6 and X3 is preferably carried out via R1 and R2;
for X2 and for X6 individually and independently R3 is a radical, whereby the radical comprises an aromatic group and is selected from the group comprising aryl, substituted aryl, heteroaryl, substituted heteroaryl, arylalkyl, substituted arylalkyl, heteroarylalkyl, substituted heteroarylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl; and
for X5 and for X7 individually and independently R3 is a radical, whereby the radical comprises an aliphatic or aromatic group and preferably is selected from the group comprising alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, heteroarylalkyl, substituted heteroarylalkyl, cycloalkylalkyl, substituted cycloalkylalkyl, heterocyclylalkyl, substituted heterocyclylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl.

5. The compound according to claim 4, characterized in that a ring is formed under participation of R3 and R4.

6. The compound according to claim 4, characterized in that for X2 and for X6 individually and independently R3 is selected from the group comprising phenyl, substituted phenyl, benzyl, substituted benzyl, 1,1-diphenylmethyl, substituted 1,1-diphenylmethyl, naphthylmethyl, substituted naphthylmethyl, thienylmethyl, substituted thienylmethyl, benzothienylmethyl, substituted benzothienylmethyl, imidazolylmethyl, substituted imidazolylmethyl, indolylmethyl and substituted indolylmethyl.

7. The compound according to any of claim 4, characterized in that for X5 and for X7 individually and independently R3 is selected from the group comprising C3-C5-alkyl, substituted C3-C5-alkyl, C5-C7-cycloalkyl, substituted C5-C7-cycloalkyl, C5-C7-cycloalkylmethyl, substituted C5-C7-cycloalkylmethyl, cycloalkylethyl, substituted cycloalkylethyl, benzyl, substituted benzyl, phenylethyl, naphthylmethyl, thienylmethyl, propenyl, propinyl, methylthioethyl, imidazolylmethyl, substituted imidazolylmethyl, indolylmethyl and substituted indolylmethyl.

8. The compound according to any of claim 1, characterized in that X1 is selected from the group comprising H, acetyl, propanoyl, butanoyl, benzoyl, fluoromethylcarbonyl, difluoromethylcarbonyl, phenyl, oxycarbonyl, methyl-oxycarbonyl, phenyl-aminocarbonyl, methyl-aminocarbonyl, phenyl-sulfonyl, 2,6-dioxo-hexahydro-pyrimidine-4-carbonyl and methyl-sulfonyl.

9. The compound according to any of claim 1, whereby
X2 is a derivative of an amino acid that is selected from the group comprising phenylalanine, 2-fluoro-phenylalanine, 3-fluoro-phenylalanine, 4-fluoro-phenylalanine, 2-chloroophenylalanine, 3-chloroophenylalanine, 4-chlorophenylalanine, 1-naphthylalanine, 2-thienylalanine, 3-thienylalanine, 3,3-diphenylalanine, tyrosine, tryptophane, histidine and respective derivatives thereof,
or X2 and X1 taken together are PhCH₂CH₂CO— or PhCH₂—;
X6 is a derivative of an amino acid that is selected from the group comprising tryptophane, phenylalanine, tyrosine, histidine, 1-naphthylalanine, benzothienylalanine, 2-aminoindan-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof,
X5 is a derivative of an amino acid, that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof, and
X7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

10. The compound according to any of claim 1, whereby X1 and/or X4 comprise one or more groups that improve water solubility, whereby the water solubility improving group is selected from the group comprising hydroxy, keto, carboxamido, ether, urea, carbamate, amino, substituted amino, guanidino, pyridyl and carboxyl.

11. A compound, preferably a C5a receptor antagonist, having the following structure: 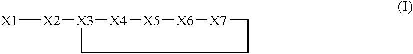
, whereby X1-X3 and X5-X7 are defined claim 1 and whereby
X4 is a cyclic or a non-cyclic amino acid, whereby the cyclic amino acid is selected from the group comprising proline, pipecolinic acid, azetidin-2-carbonic acid, tetrahydroisochinoline-3-carbonic acid, tetrahydroisochinoline-1-carbonic acid, octahydroindole-2-carbonic acid, 1-aza-bicyclo-[3.3.0]-octane-2-carbonic acid, 4-phenyl-pyrrolidine-2-carbonic acid, cis-Hyp and trans-Hyp, and the non-cyclic amino acid is selected from the group comprising Ser, Gln, Asn, Cys(O₂CH₂CH₂CONH₂), Arg, Hyp(COCH₂OCH₂CH₂OCH₂CH₂OCH₃), Hyp(CONH—CH₂CH(OH)—CH₂OH) and respective derivatives thereof and respective analogs thereof, and
the lines—in formula (I) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

12. The compound according to claim 11, characterized in that the amino acid represented by X4 is preferably selected from the group comprising proline, pipecolinic acid, azetidin-2-carbonic acid, tetrahydroisochinoline-3-carbonic acid, tetrahydroisochinoline-1-carbonic acid, octahydroindole-2-carbonic acid, 1-aza-bicyclo-[3.3.0]-octane-2-carbonic acid, 4-phenyl-pyrrolidine-2-carbonic acid, Hyp, Ser, Gln, Asn, Cys(O₂CH₂CH₂CONH₂) and Arg.

13. The compound according to claim 11, whereby
X2 is a derivative of an amino acid that is selected from the group comprising phenylalanine, 2-fluoro-phenylalanine, 3-fluoro-phenylalanine, 4-fluoro-phenylalanine, 2-chlorophenylalanine, 3-chlorophenylalanine, 4-chlorophenylalanine, 1-naphthylalanine, 2-thienylalanine, 3-thienylalanine, 3,3-diphenylalanine, tyrosine, tryptophane, histidine and respective derivatives thereof,
or X2 and X1 taken together are PhCH₂CH₂CO— or PhCH₂—;
X6 is a derivative of an amino acid that is selected from the group comprising tryptophane, phenylalanine, tyrosine, histidine, 1-naphthylalanine, benzothienylalanine, 2-aminoindan-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof,
X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof; and
X7 is a derivative of an amino acid that is selected from the group comprising norvaline, norleucine, homo-leucine, leucine, isoleucine, Valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

14. A compound, preferably a C5a receptor antagonist, having the following structure: 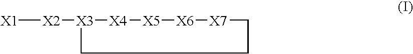
, whereby X1-X2 and X4-X7 are defined as in claim 1 and whereby X3 has the following structure 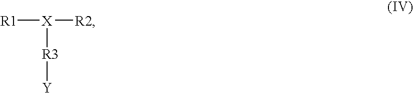
whereby
X is C(R4) or N,
R1 is optionally present and if R1 is present then R1 is a radical, which is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl;
R2 is optionally present and if R2 is present then R2 is a radical that is selected from the group comprising >C═O, >C═S, >SO₂, >PO(OH), >B(OH), >CH₂, >CH₂CO, >CHF and >CF₂;
R4 is a radical, whereby the radical is selected from the group comprising H, F, CF₃, alkyl and substituted alkyl;
the bond of structure (IV) to the moieties X2 and X4 preferably takes place via R1 and R2;
R3 is a radical, whereby the radical is selected from the group comprising H, alkyl, substituted alkyl, cycloalkyl, substituted cycloalkyl, heterocyclyl, substituted heterocyclyl, aryl, substituted aryl, heteroaryl, substituted heteroaryl, cycloalkylalkyl, substituted cycloalkylalkyl, heterocyclylalkyl, substituted heterocyclylalkyl, arylalkyl, substituted arylalkyl, heteroarylalkyl and substituted heteroarylalkyl;
Y is optionally present and if Y is present then Y is a radical that is selected from the group comprising —N(YB)—, —O—, —S—, —S—S—, —CO—, —C═N—O—, —CO—N(YB)— and 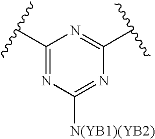
, whereby YB, YB 1 and YB2 are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl.

15. The compound according to claim 14, characterized in that R3 is a radical, whereby the radical is selected from the group comprising methyl, ethyl, propyl, butyl, benzyl and 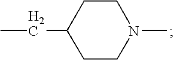
Y is optionally present and if Y is present then Y is a radical, whereby the radical is selected from the group comprising —N(YB)—, —O—, —S— and —S—S—, and YB is preferably defined as in claim 14.

16. The compound according to claim 14, whereby
X2 is a derivative of an amino acid that is selected from the group comprising phenylalanine, 2-fluoro-phenylalanine, 3-fluoro-phenylalanine, 4-fluoro-phenylalanine, 2-chlorophenylalanine, 3-chlorophenylalanine, 4-chlorophenylalanine, 1-naphthylalanine, 2-thienylalanine, 3-thienylalanine, 3,3-diphenylalanine, tyrosine, tryptophane, histidine and respective derivatives thereof,
or X2 and X1 taken together are PhCH₂CH₂CO— or PhCH₂—;
X6 is a derivative of an amino acid that is selected from the group comprising tryptophane, phenylalanine, tyrosine, histidine, 1-naphthylalanine, benzothienylalanine, 2-aminoindane-2-carbonic acid, 2-thienylalanine, 3-thienylalanine, 2-fluoro-phenylalanine, 3-fluoro-phenylalanine, 4-fluoro-phenylalanine, 2-chlorophenylalanine, 3-chlorophenylalanine, 4-chlorophenylalanine and respective derivatives thereof,
X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carbonic acid, 2-methyl-D-phenylalanine and respective derivatives thereof, and
X7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

17. The compound according to claim 1, characterized in that X3 is a derivative of an amino acid that is selected from the group comprising alpha-amino glycine, alpha-beta-diaminopropionic acid (Dap), alpha-gamma-diaminobutyric acid (Dab), ornithine, lysine, homolysine, Phe(4-NH2), 2-amino-3-(4-piperidinyl)propionic acid and 2-amino-3-(3-piperidinyl)propionic acid, and the amino acid is modified at the side chain.

18. A compound, preferably a C5a receptor antagonist, preferably according to claim 1, having the following structure: 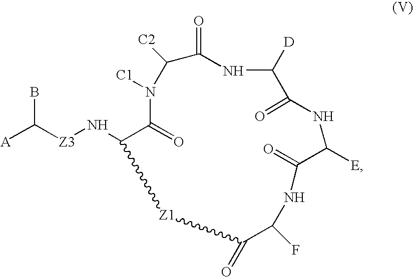
, whereby
A is selected from the group comprising H, NH₂, NHalkyl, Nalkyl2, NHacyl and OH,
B is selected from the group comprising CH2(aryl), CH(aryl)₂, CH2(heteroaryl), substituted CH2(aryl), aryl, substituted aryl and heteroaryl,
C1 and C2 are individually and independently selected from the group comprising alkyl and substituted alkyl, whereby a bond can optionally be formed between C1 and C2,
D is selected from the group comprising alkyl, cycloalkyl, CH2(cycloalkyl), CH2CH2(cycloalkyl), CH2Ph(2-Me) and CH2-S-alkyl,
E is selected from the group comprising CH2(aryl), substituted CH2(aryl) and CH2(heteroaryl),
F is selected from the group comprising alkyl, CH2-S-alkyl, CH2CH2-S-Me, CH2CH═CH2, CH—CCH, cyclohexyl, CH2cyclohexyl, CH2Ph, CH2naphthyl, and CH2-thienyl,
Z1 is selected from the group comprising (CH2)nNH with n=1, 2, 3, 4, (CH2)3O, (CH2)2O, (CH2)4, (CH2)3, CH2Ph(4-NH) and CH2(4-piperidinyl), and
Z3 is optionally present and if Z3 is present then Z3 is selected from the group comprising CO and CH2.

19. The compound according to claim 18, characterized in that
A is selected from the group comprising H, NH2, NHEt, NHAc, and OH,
B is selected from the group comprising CH2Ph, CH2Ph(4-F), CH(Ph)₂, CH2thienyl, CH2naphthyl, phenyl, Ph(4-F) and thienyl,
C1 is selected from the group comprising H and methyl, C2 is selected from the group comprising methyl and CH₂OH, or if C1 and C2 are connected by a bond, the resulting structure is selected from the group comprising —(CH2)2-, —(CH2)3-, —(CH2)4- and —CH2CH(OH)CH2-.
D is selected from the group comprising CH2CH2iPr, CH2iPr, cyclohexyl, CH2cyclohexyl, CH2CH2cyclohexyl, CH2Ph(2-Me), CH2-S-tBu and CH2-S-iPr,
E is selected from the group comprising CH2Ph, CH2Ph(2-Cl), CH2Ph(3-Cl), CH2Ph(4-Cl), CH2Ph(2-F), CH2Ph(3-F), CH2Ph(4-F), CH2indolyl, CH2thienyl, CH2benzothienyl and CH2naphthyl,
F is selected from the group comprising (CH2)3CH3, (CH2)2CH3, (CH2)-2-iPr, CH2-iPr, iPr, CH2-S-Et, CH2CH2-S-Me, CH2CH═CH2, CH2-CCH and cyclohexyl,
Z1 is selected from the group comprising (CH2)nNH with n=1, 2, 3, 4, (CH2)3O, CH2Ph(4-NH) and CH2(4-piperidinyl), and
Z3 is optionally present, and if Z3 is present, then Z3 is selected from the group comprising CO and CH2.

20. A compound, preferably a C5a receptor antagonist, whereby the compound has the following structure: 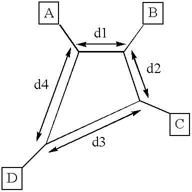
whereby d1, d2, d3 and d4 constitute the distances of A, B, C and D in at least one energetically accessible conformer of the compound and have the following values:
d1=5.1±1.0 Å
d2=11.5±1.0 Å
d3=10.0±1.5 Å
d4=6.9±1.5 Å
A and C are individually and independently a hydrophobic radical, whereby the hydrophobic radical is selected from the group comprising alkyl, cycloalkyl, heterocyclyl, aryl and heteroaryl;
B and D are individually and independently an aromatic or a heteroaromatic radical, whereby the aromatic radical preferably is aryl, and preferably the heteroaromatic radical is heteroaryl.

21. The compound according to claim 20, whereby A and C are individually and independently selected from the group comprising C3-C6-alkyl, C5-C7-cycloalkyl, methylthioethyl, methylthio-tert-butyl, indolyl, phenyl, naphthyl, thienyl, propenyl, propinyl, hydroxyphenyl, indolyl and imidazolyl;
B is selected from the group comprising phenyl, substituted phenyl, naphthyl, thienyl, benzothienyl, hydroxyphenyl, indolyl, and imidazolyl; and
D is selected from the group comprising phenyl, naphthyl, thienyl, thiazolyl, furanyl, hydroxyphenyl, indolyl and imidazolyl.

22. A compound, preferably a C5a receptor antagonist, having the following structure: 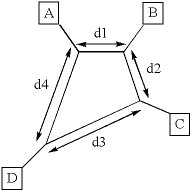
, whereby
A, B, C and D constitute the C-alpha atoms in amino acids, amino acid analogs or amino acid derivatives,
d1, d2, d3 and d4 constitute the distances of A, B, C and D in at least one energetically accessible conformer of the compound and have the following values:
d1=3.9±0,5 Å
d2=3.9±0,5 Å
d3=9.0±1,5 Å
d4=9.0±1,5 Å;
whereby the amino acids, whose alpha-atoms are constituted by A and C, individually and independently have a hydrophobic amino acid side chain that incorporates an alkyl-, cycloalkyl, cycloalkylalkyl, heterocyclyl, aryl, arylalkyl, heteroaryl, heteroarylalkyl or methylthio-tert-butyl group;
whereby the amino acids, whose alpha-atoms are constituted by B and D, individually and independently have an aromatic or heteroaromatic amino acid side chain that comprises an aryl, arylalkyl, heteroaryl or heteroarylalkyl group.

23. The compound according to claim 22,
whereby the amino acid, whose alpha-atom is constituted by A, is selected from the group comprising C3-C6-alkyl, methylthioethyl, propenyl, propinyl, R5, methyl-R5 and ethyl-R5, whereby R5 is a radical that is selected from the group comprising C5-C7-cycloalkyl, phenyl, substituted phenyl, hydroxyphenyl, indolyl, imidazolyl, naphthyl and thienyl;
whereby the amino acid whose alpha-atom is constituted by B, is selected from the group comprising R5, methyl-R5 and ethyl-R5, whereby R5 is a radical that is selected from the group comprising phenyl, substituted phenyl, naphthyl, thienyl, benzothienyl, hydroxyphenyl, indolyl and imidazolyl;
whereby the amino acid whose alpha-atom is constituted by C, is selected from the group comprising C3-C6-alkyl, R5, methyl-R5 and ethyl-R5, whereby R5 is a radical that is selected from the group comprising C5-C7-cycloalkyl, phenyl, 1-methyl-phenyl, 2-methyl-phenyl, 3-methyl-phenyl and S-tBu; and
whereby the amino acid whose alpha-atom is constituted by D, is selected from the group comprising R5, methyl-R5 and ethyl-R5, whereby R5 is a radical that is selected from the group comprising phenyl, naphthyl, thienyl, thiazolyl, furanyl, hydroxyphenyl, indolyl and imidazolyl.

24. A compound, preferably a C5a receptor antagonist, having the following structure:
X1-X2-X3-X4-X5-X6-X7-X8 (II)
, whereby
X1 is a radical having a mass of about 1-300, whereby X1 is preferably selected from the group comprising R5-, R5-CO—, R5-N(R6)-CO—, R5-O—CO—, R5-SO₂—, R5-N(R6)-SO₂—, R5-N(R6)-, R5-N(R6)-CS—, R5-N(R6)-C(NH)—, R5-CS—, R5-P(O)OH—, R5-B(OH)—, R5-CH═N—O—CH₂—CO—, whereby R5 and R6 are individually and independently selected from the group comprising H, F, hydroxy, alkyl, substituted alkyl, cycloalkyl, substituted cycloalkyl, heterocyclyl, substituted heterocyclyl, arylalkyl, substituted arylalkyl, aryl, substituted aryl, heteroaryl, substituted heteroaryl, acyl, substituted acyl, alkoxy, alkoxyalkyl, substituted alkoxyalkyl, aryloxyalkyl and substituted aryloxyalkyl,
X2 is a radical that mimics the biological binding characteristics of a phenylalanine unit,
X3 and X4 are individually and independently a spacer, whereby the spacer is preferably selected from the group comprising amino acids, amino acid analogs and amino acid derivates,
X5 is a radical that mimics the biological binding characteristics of a cyclohexylalanine or homoleucine unit,
X6 is a radical that mimics the biological binding characteristics of a tryptophan unit,
X7 is a radical that mimics the biological binding characteristics of a norleucine or phenylalanine unit,
X8 is a radical which is optionally present in structure II, and if present it is selected from the group comprising H, NH₂, OH, NH—OH, NH-Oalkyl, amino, substituted amino, alkoxy, substituted alkoxy, hydrazino, substituted hydrazino, aminooxy, substituted aminooxy, alkyl, substituted alkyl, cycloalkyl, substituted cycloalkyl, heterocyclyl, substituted heterocyclyl, heteroaryl, substituted heteroaryl, arylalkyl, substituted arylalkyl, aryl, substituted aryl, amino acid, amino acid derivative and amino acid analogon;
the connecting lines—in formula (II) indicate chemical bonds, whereby the chemical bond is individually and independently selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

25. The compound according to claim 24, in which
X1 is a radical having a mass of about 1-300, whereby the radical is preferably selected from the group comprising R5, R5-CO—, R5-N(R6)-CO—, R5-O—CO—, R5-SO₂—, R5-N(R6)-C(NH)—, whereby preferably R5 and R6 are individually and independently selected from the group comprising H, alkyl, substituted alkyl, cycloalkyl, substituted cycloalkyl, heterocyclyl, substituted heterocyclyl, aryl and substituted aryl;
X2 and X6 are individually and independently an aromatic amino acid, a derivative or an analogon thereof,
X5 and X7 are individually and independently a hydrophobic amino acid, a derivative or an analogon thereof.

26. The compound according to claim 24, whereby X2, X5, X6 and X7 individually and independently have the following structure: 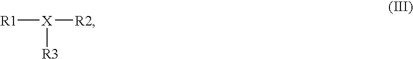
whereby
X is C(R4) or N,
R1 is optionally present and if R1 is present R1 is a radical, that is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, cycloalkylalkyl and substituted cycloalkylalkyl;
R2 is optionally present and if R2 is present than R2 is a radical, that is selected from the group comprising >C═O, >C═S, >SO₂, >S═O, >C═NH, >C═N—CN, >PO(OH), >B(OH), >CH₂, >CH₂CO, >CHF and >CF₂;
R4 is a radical, whereby the radical is selected from the group comprising H, F, CH₃, CF₃, alkyl and substituted alkyl;
and the binding of structure (III) to the moieties X1 and X3, X4 and X6, X5 and X7, and X6 and X8 preferably takes place via R1 and R2;
for X2 and for X6 individually and independently R3 is a radical, whereby the radical comprises an aromatic group and is selected from the group comprising aryl, substituted aryl, heteroaryl, substituted heteroaryl, arylalkyl, substituted arylalkyl, heteroarylalkyl, substituted heteroarylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl; and
for X5 and for X7 individually and independently R3 is a radical, whereby the radical comprises an aliphatic or aromatic group and is preferably selected from the group comprising alkyl, substituted alkyl, cycloalkyl, substituted cycloalkyl, heterocyclyl, substituted heterocyclyl, aryl, substituted aryl, heteroaryl, substituted heteroaryl, arylalkyl, substituted arylalkyl, heteroarylalkyl, substituted heteroarylalkyl, cycloalkylalkyl, substituted cycloalkylalkyl, heterocyclylalkyl, substituted heterocyclylalkyl, alkyloxy-alkyl, substituted alkyloxy-alkyl, alkyloxy-cycloalkyl, substituted alkyloxy-cycloalkyl, alkyloxy-heterocyclyl, substituted alkyloxy-heterocyclyl, alkyloxy-aryl, substituted alkyloxy-aryl, alkyloxy-heteroaryl, substituted alkyloxy-heteroaryl, alkylthio-alkyl, substituted alkylthio-alkyl, alkylthio-cycloalkyl and substituted alkylthio-cycloalkyl.

27. The compound according to claim 26, characterized in that a ring is formed under participation of R3 and R4.

28. The compound according to claim 26, characterized in that for X2 and for X6 individually and independently R3 is selected from the group comprising phenyl, substituted phenyl, benzyl, substituted benzyl, 1,1-diphenylmethyl, substituted 1,1-diphenylmethyl, naphthylmethyl, substituted naphthylmethyl, thienylmethyl, substituted thienylmethyl, benzothienylmethyl, substituted benzothienylmethyl, imidazolylmethyl, substituted imidazolylmethyl, indolylmethyl and substituted indolylmethyl.

29. The compound according to claim 24, characterized in that for X5 and for X7 individually and independently R3 is selected from the group comprising C3-C5-alkyl, substituted C3-C5-alkyl, C5-C7-cycloalkyl, substituted C5-C7-cycloalkyl, C5-C7-cycloalkylmethyl, substituted C5-C7-cycloalkylmethyl, cycloalkylethyl, substituted cycloalkylethyl, benzyl, substituted benzyl, phenylethyl, naphthylmethyl, thienylmethyl, propenyl, propinyl, methylthioethyl, imidazolylmethyl, substituted imidazolylmethyl, indolylmethyl and substituted indolylmethyl.

30. The compound according to claim 24, characterized in that X8 is selected from the group comprising H, OR1 and NR1R2, whereby R1 and R2 are individually and independently selected from the group comprising H, alkyl, aryl, cycloalkyl and arylalkyl.

31. The compound according to claim 24, characterized in that X1 is selected from the group comprising H, acetyl, propanoyl, butanoyl, benzoyl, fluoromethylcarbonyl, difluoromethylcarbonyl, phenyl, oxycarbonyl, methyl-oxycarbonyl, phenyl-aminocarbonyl, methyl-aminocarbonyl, phenyl-sulfonyl, 2,6-dioxo-hexahydro-pyrimidine-4-carbonyl and methyl-sulfonyl.

32. The compound according to claim 24, whereby X1 and/or X4 comprise one or more groups that improve water solubility, whereby the water solubility improving group is selected from the group comprising hydroxy, keto, carboxamido, ether, urea, carbamate, amino, substituted amino, guanidino, pyridyl and carboxyl.

33. A compound, preferably a C5a receptor antagonist, having the following structure:
X1-X2-X3-X4-X5-X6-X7-X8 (II)
, whereby X1-X3 and X5-X8 are defined claim 24 and whereby
X4 is a cyclic or a non-cyclic amino acid, whereby the cyclic amino acid is selected from the group comprising proline, pipecolic acid, azetidine-2-carbonic acid, tetrahydroisoquinoline-3-carboxylic acid, tetrahydroisoquinoline-1-carboxylic acid, octahydroindole-2-carboxylic acid, 1-aza-bicyclo-[3.3.0]-octane-2-carboxylic acid, 4-phenyl-pyrrolidine-2-carboxylic acid, cis-Hyp and trans-Hyp, and the non-cyclic amino acid is selected from the group comprising Ser, Gln, Asn, Cys(O₂CH₂CH₂CONH₂), Arg, Hyp(COCH₂OCH₂CH₂OCH₂CH₂OCH₃), Hyp(CONH—CH₂CH(OH)—CH₂OH) and respective derivatives thereof and respective analogs thereof, and
the connecting lines—in formula (I) indicate chemical bonds, whereby the chemical bond individually and independently is preferably selected from the group comprising covalent bonds, ionic bonds and coordinative bonds, whereby preferably the bond is a chemical bond and more preferably the chemical bond is a bond selected from the group comprising amide bonds, disulfide bonds, ether bonds, thioether bonds, oxime bonds and aminotriazine bonds.

34. The compound according to claim 33, characterized in that the amino acid represented by X4 is preferably selected from the group comprising proline, pipecolic acid, azetidine-2-carboxylic acid, tetrahydroisoquinoline-3-carboxylic acid, tetrahydroisoquinoline-1-carboxylic acid, octahydroindole-2-carboxylic acid, 1-aza-bicyclo-[3.3.0]-octane-2-carboxylic acid, 4-phenyl-pyrrolidine-2-carboxylic acid, Hyp, Ser, Gln, Asn, Cys(O₂CH₂CH₂CONH₂) and Arg.

35. A compound, preferably a C5a receptor antagonist, having the following structure:
X1-X2-X3-X4-X5-X6-X7-X8 (II)
whereby X1-X2 and X4-X8 are defined as in claim 24 and whereby
X3 has the following structure: 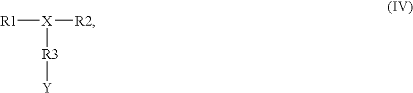
whereby
X is C(R4) or N,
R1 is optionally present and if R1 is present then R1 is a radical which is selected from the group comprising >N—R1B, >C(R1B)(R1D) and >O, whereby R1B and R1D are individually and independently selected from the group comprising H, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl;
R2 is optionally present and if R2 is present then R2 is a radical which is selected from the group comprising >C═O, >C═S, >SO₂, >PO(OH), >B(OH), >CH₂, >CH₂CO, >CHF and >CF₂;
R4 is a radical, whereby the radical is selected from the group comprising H, F, CF₃, alkyl and substituted alkyl;
the binding of structure (IV) to the moieties X2 and X4 preferably takes place via R1 and R2;
R3 is a radical selected from the group comprising H, alkyl, substituted alkyl, cycloalkyl, substituted cycloalkyl, cycloalkylalkyl, substituted cycloalkylalkyl, heterocyclyl, substituted heterocyclyl, heterocyclylalkyl, substituted heterocyclylalkyl, aryl, substituted aryl, arylalkyl, substituted arylalkyl, heteroaryl, substituted heteroaryl, heteroarylalkyl, substituted heteroarylalkyl, acyl, substituted acyl, alkoxyalkyl, substituted alkoxyalkyl, aryloxyalkyl, substituted aryloxyalkyl, sulfhydrylalkyl, substituted sulfhydrylalkyl, hydroxyalkyl, substituted hydroxyalkyl, carboxyalkyl, substituted carboxyalkyl, carboxamidoalkyl, substituted carboxamidoalkyl, carboxyhydrazinoalkyl, ureidoalkyl aminoalkyl, substituted aminoalkyl, guanidinoalkyl and substituted guanidinoalkyl;
Y is optionally present and if Y is present then Y is a radical that is selected from the group comprising H, —N(YB1)-C0-YB2, —N(YB1)-CO—N(YB2)(YB3), —N(YB1)-C(N—YB2)-N(YB3)(YB4), —N(YB1)(YB2), —N(YB1)-SO₂—YB2, O—YB1, S—YB1, —CO—YB1, —CO—N(YB1)(YB2) and —C═N—O—YB1, whereby YB1, YB2, YB3 and YB4 are individually and independently selected from the group comprising H, CN, NO₂, alkyl, substituted alkyl, cycloalkyl, substituted cycloalkyl, heterocyclyl, substituted heterocyclyl, aryl, substituted aryl, heteroaryl, substituted heteroaryl, arylakyl, substituted arylalkyl, cycloalkylalkyl and substituted cycloalkylalkyl.

36. The compound according to claim 35, characterized in that
R3 is a radical having the structure
—(CH₂)ₘ—Y (VII)
or
—(CH₂)ₘ—C6H₄—Y (VIII) ist
, whereby
m is 1, 2, 3 or 4;
Y is N(R3b)(R3c) or —N(YB1)-C(N—YB2)-N(YB3)(YB4), whereby R3b, R3c, YB1, YB2, YB3 and YB4 are individually and independently selected from the group comprising H, CN and alkyl.

37. The compound according to claim 35, characterized in that a ring is formed between two moieties of the compound, whereby the moieties of the compound are individually and independently selected from the group comprising YB1, YB2, YB3 and YB4.

38. The compound according to claim 37, characterized in that the ring is formed under participation of YB2 and YB3.

39. The compound according to claim 35, characterized in that Y is —NH₂
or

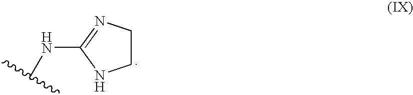

40. A compound according to claim 24, whereby
X2 is a derivative of an amino acid that is selected from the group comprising phenylalanine, 2-fluoro-phenylalanine, 3-fluoro-phenylalanine, 4-fluoro-phenylalanine, 2-chloro-phenylalanine, 3-chloro-phenylalanine, 4-chloro-phenylalanine, 1-naphthylalanine, 2-thienylalanine, 3-thienylalanine, 3,3-diphenylalanine, tyrosine, tryptophane, histidine and respective derivatives thereof,
or X2 and X1 together are PhCH₂CH₂CO— or PhCH₂—;
X6 is a derivative of an amino acid that is selected from the group comprising tryptophane, phenylalanine, tyrosine, histidine, 1-naphthylalanine, benzothienylalanine, 2-aminoindane-2-carboxylic acid, 2-thienylalanine, 3-thienylalanine, 2-fluoro-phenylalanine, 3-fluoro-phenylalanine, 4-fluoro-phenylalanine, 2-chloro-phenylalanine, 3-chloro-phenylalanine, 4-chloro-phenylalanine and respective derivatives thereof;
X5 is a derivative of an amino acid that is selected from the group comprising D-cyclohexylalanine, D-cyclohexylglycine, D-homo-cyclohexylalanine, D-homoleucine, D-cysteine(tBu), D-cysteine(iPr), octahydroindole-2-carboxylic acid, 2-methyl-D-phenylalanine and respective derivatives thereof; and
X7 is a derivative of an amino acid that is selected from the group comprising norvaline, norleucine, homo-leucine, leucine, isoleucine, valine, cysteine, cysteine(Me), cysteine(Et), cysteine(Pr), methionine, allylglycine, propargylglycine, cyclohexylglycine, cyclohexylalanine, phenylalanine, tyrosine, tryptophane, histidine, 1-naphthylalanine, 2-thienylalanine, 3-thienylalanine and respective derivatives thereof.

41. The compound according to claim 1, characterized in that X3 is an amino acid derivative of an amino acid, whereby the amino acid is selected from the group comprising alpha-amino-glycine, alpha-beta-diaminopropionic acid (Dap), alpha-gamma-diaminobutanoic acid (Dab), ornithine, lysine, homolysine, Phe(4-NH2), 2-amino-3-(4-piperidinyl)propionic acid and 2-amino-3-(3-piperidinyl)propionic acid, and the amino acid is derivatized at the side chain.

42. A compound, preferably a C5a receptor antagonist, preferably according to claim 1, having the following structure: 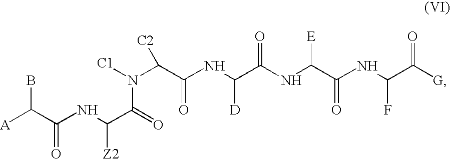
, whereby
A is selected from the group comprising H, NH₂, NHalkyl, Nalkyl₂, NHacyl, substituted NHacyl and OH,
B is selected from the group comprising CH₂(aryl), CH(aryl)₂, CH₂(heteroaryl) and substituted CH₂(aryl), C1 and C2 are individually and independently selected from the group comprising alkyl and substituted alkyl, whereby optionally a bond can be formed between C1 and C2,
D is selected from the group comprising alkyl, cycloalkyl, CH₂(cycloalkyl), CH₂CH₂(cycloalkyl), CH₂Ph(2-Me) and CH₂—S-alkyl,
E is selected from the group comprising CH₂(aryl), substituted CH₂(aryl) and CH₂(heteroaryl),
F is selected from the group comprising alkyl, CH₂—S-alkyl, CH₂CH₂—S-Me, CH₂CH═CH₂, CH—CCH, cyclohexyl, CH₂cyclohexyl, CH₂Ph, CH₂naphthyl, and CH₂thienyl, and
Z2 is —R3-Y—, whereby R3 is selected from the group comprising H, alkyl, and arylalkyl, and Y is optionally present, and if Y is present, Y is selected from the group comprising H, N(YB1)(YB2), N(YB1)C(N—YB2)-N(YB3)(YB4), 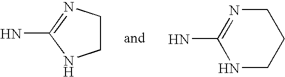
, whereby YB1, YB2, YB3 and YB4 are individually and independently selected from the group comprising H, CN and alkyl and optionally a ring is formed under participation of at least two of YB1, YB2, YB3 and YB4, and
G is selected from the group comprising H, OR1 and NR1R2, whereby R1 and R2 are individually and independently selected from the group comprising H, alkyl, aryl, cycloalkyl and arylalkyl.

43. The compound according to claim 42, characterized in that
A is selected from the group comprising H, NH₂, NHEt, NHAc, and OH,
B is selected from the group comprising CH₂Ph, CH₂Ph(4-F), CH(Ph)₂, CH₂thienyl and CH₂naphthyl,
C1 is selected from the group comprising H and methyl, C2 is selected from the group comprising methyl and CH₂OH, or if C1 and C2 are connected by a bond, the resulting structure is selected from the group comprising —(CH₂)₂—, —(CH₂)₃—, —(CH₂)₄— and —CH₂CH(OH)CH₂—.
D is selected from the group comprising CH₂CH₂iPr, CH₂iPr, cyclohexyl, CH₂cyclohexyl, CH₂CH₂cyclohexyl, CH₂Ph(2-Me), CH₂—S-tBu and CH₂—S-iPr,
E is selected from the group comprising CH₂Ph, CH₂Ph(2-Cl), CH₂Ph(3-Cl), CH₂Ph(4-Cl), CH₂Ph(2-F), CH₂Ph(3-F), CH₂Ph(4-F), CH₂indolyl, CH₂thienyl, CH₂benzothienyl and CH₂naphthyl,
F is selected from the group comprising (CH₂)₃CH₃, (CH₂)₂CH₃, (CH₂)₂-iPr, CH₂-iPr, iPr, CH₂—S-Et, CH₂CH₂—S-Me, CH₂CH═CH₂, CH₂—CCH and cyclohexyl,
Z2 is —R3-Y—, whereby R3 is selected from the group comprising CH₂, (CH₂)₂, (CH₂)₃, (CH₂)₄ and CH₂—C6H₄, and Y is selected from the group comprising NH₂, NHEt, N(Et)₂, NH—C(NH)—NH₂ and 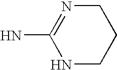
and
G is selected from the group comprising NH₂, NHMe, OH, and H.

44. The compound according to claim 1, whereby the compound is one of the following compounds:

| No. | Compound |
|---|---|
| 1 | Ac-Phe-[Orn-Pro-cha-Trp-Phe] |
| 2 | Ac-Phe-[Orn-Hyp-cha-Trp-Phe] |
| 3 | HOCH₂(CHOH)₄—C═N—O—CH₂—CO-Phe-[Orn-Pro-cha-Trp- |
| | Nle] |
| 4 | X-Phe-[Orn-Pro-cha-Trp-Nle]; X = 2-acetamido-1-methyl- |
| | glucuronyl |
| 5 | Ac-Phe-[Orn-Hyp(COCH₂OCH₂CH₂OCH₂CH₂OCH₃)-cha- |
| | Trp-Nle] |
| 6 | Ac-Phe-[Orn-Hyp(CONH—CH₂CH(OH)—CH₂OH)-cha-Trp- |
| | Nle] |
| 20 | Ac-Phe-[Orn-Pro-cha-Trp-Ecr] |
| 28 | Ac-Phe-[Orn-Pro-cha-Trp-Nle] |
| 29 | Ac-Phe-[Orn-Pro-cha-Trp-Met] |
| 31 | Ac-Phe-[Orn-Pro-cha-Trp-Nva] |
| 32 | Ac-Phe-[Orn-Pro-cha-Trp-Hle] |
| 33 | Ac-Phe-[Orn-Pro-cha-Trp-Eaf] |
| 34 | Ac-Phe-[Orn-Pro-cha-Trp-Ebd] |
| 35 | Ac-Phe-[Orn-Pro-cha-Trp-Eag] |
| 36 | Ac-Phe-[Orn-Pro-cha-Trp-Pmf] |
| 37 | Ac-Phe-[Orn-Pro-cha-Trp-2Ni] |
| 38 | Ac-Phe-[Orn-Pro-cha-Trp-Thi] |
| 41 | Ph-CH₂—CH₂—CO-[Orn-Pro-cha-Trp-Nle] |
| 42 | H-Phe-[Orn-Pro-cha-Trp-Nle] |
| 43 | Ac-Lys-Phe-[Orn-Pro-cha-Trp-Nle] |
| 44 | H-Phe-[Orn-Ser-cha-Trp-Nle] |
| 51 | Ac-Phe-Orn-Pro-cha-Trp-Phe-NH₂ |
| 52 | Ac-Phe-Orn-Aze-cha-Bta-Phe-NH₂ |
| 53 | Ac-Phe-Orn-Pro-cha-Bta-2Ni—NH₂ |
| 54 | Ac-Phe-Orn-Pro-cha-Bta-Cha-NH₂ |
| 55 | Ac-Phe-Orn-Pip-cha-Trp-Phe-NH₂ |
| 56 | Ph-CH₂-[Orn-Pro-cha-Trp-Nle] |
| 57 | Ph-CH₂-[Orn-Pro-cha-Trp-Phe] |
| 58 | Ac-Phe-[Orn-Pro-cha-Trp-1Ni] |
| 59 | Ph-CH(OH)—CH₂—CO-[Orn-Pro-cha-Trp-Nle] |
| 61 | Ac-Phe-Orn-Pro-cha-Trp-Phe-NH₂ |
| 62 | Ac-Phe-Orn-Pro-cha-Bta-Phe-NH₂ |
| 64 | Ac-Phe-Orn-Pro-cha-Trp-2Ni—NH₂ |
| 65 | Ac-Phe-Orn-Pro-cha-Trp-Cha-NH₂ |
| 66 | Ac-Thi-Orn-Aze-cha-Bta-Phe-NH₂ |
| 67 | Ac-Thi-Orn-Pip-cha-Bta-Phe-NH₂ |
| 68 | Ac-Phe-Orn-Pro-cha-Trp-Eap-NH₂ |
| 69 | Me₂-Phe-Orn-Pro-cha-Trp-Phe-NH₂ |
| 70 | Ph₂-CH—CH₂—CO-Orn-Pro-cha-Trp-Phe-NH₂ |
| 71 | Ac-Ebw-Orn-Pro-cha-Trp-Phe-NH₂ |
| 72 | Ac-Phe-Orn-Pro-cha-Trp-NH—CH₂—CH₂-Ph |
| 73 | Ac-Phe-Orn-Aze-cha-Bta-NH—CH₂—CH₂-Ph |
| 74 | H-Phe-Orn-Pro-cha-Trp-Phe-NH₂ |
| 75 | H-Me-Phe-Orn-Pro-cha-Trp-Phe-NH₂ |
| 76 | Bu-NH—CO-Phe-Orn-Pro-cha-Trp-Phe-NH₂ |
| 77 | Ac-Thi-Orn-Pro-cha-Trp-Phe-NH₂ |
| 78 | Ac-Ebw-Orn-Pro-cha-Trp-Phe-NH₂ |
| 79 | Ac-Phe-Orn-Ala-cha-Trp-Phe-NH₂ |
| 80 | Ac-Phe-Orn-Pro-cha-Trp-Thi-NH₂ |
| 81 | Ac-Phe-Orn-Aze-cha-Pcf-Phe-NH₂ |
| 82 | Ac-Phe-Orn(Ac)-Pro-cha-Trp-Phe-NH₂ |
| 83 | Ac-Phe-Orn-Aze-cha-Trp-Phe-NH₂ |
| 84 | Ac-Phe-Trp-Pro-cha-Trp-Phe-NH₂ |
| 85 | Ph-NH—CO-Phe-Orn-Pro-cha-Trp-Phe-NH₂ |
| 86 | Bu-O—CO-Phe-Orn-Pro-cha-Trp-Phe-NH₂ |
| 87 | Ac-Phe-Lys-Pro-cha-Trp-Phe-NH₂ |
| 88 | Ac-Phe-Arg-Pro-cha-Trp-Phe-NH₂ |
| 89 | Ac-Phe-Gln-Pro-cha-Trp-Phe-NH₂ |
| 92 | Ac-Phe-Orn-Pip-cha-Trp-Phe-NH₂ |
| 93 | Ac-Phe-Orn-Hyp-cha-Trp-Phe-NH₂ |
| 94 | Ac-Phe-Orn-Pro-cha-Trp-1Ni—NH₂ |
| 95 | Ac-Phe-Orn-Aze-cha-Bta-Phe-NH-Me |
| 96 | CH₃—SO₂-Phe-Orn-Aze-cha-Bta-Phe-NH₂ |
| 99 | Ac-Phe-Orn-Aze-cha-Pff-Phe-NH₂ |
| 100 | Ac-Phe-Orn-Aze-cha-Mcf-Phe-NH₂ |
| 101 | Ac-Phe-Orn(Ac)-Aze-cha-Bta-Phe-NH₂ |
| 102 | Ac-Ebw-Orn-Pro-cha-Trp-Phe-NH₂ |
| 103 | Ac-Phe-Trp-Pro-cha-Trp-Phe-NH₂ |
| 104 | Ac-Phe-Arg-Pro-cha-Trp-Phe-NH₂ |
| 105 | Ac-Phe-Orn-Pip-cha-Trp-Phe-NH₂ |
| 106 | 3PP-Orn-Aze-cha-Bta-Phe-NH₂ |
| 107 | Ac-Phe-Orn-Tic-cha-Trp-Phe-NH₂ |
| 108 | Ac-Phe-Orn-Ser-cha-Trp-Phe-NH₂ |
| 109 | Ac-Phe-Orn-Pro-chg-Trp-Phe-NH₂ |
| 110 | Ac-Phe-Orn-Pro-hch-Trp-Phe-NH₂ |
| 111 | Ac-Phe-Orn-Pro-cha-Trp-Phg-NH₂ |
| 112 | Ac-Phe-Bta-Aze-cha-Bta-Phe-NH₂ |
| 113 | Ac-Phe-Trp-Pro-cha-Bta-Phe-NH₂ |
| 115 | Ac-Phe-Orn-Pip-cha-Trp-Phe-OH |
| 116 | Ac-Phe-Orn-Tic-cha-Trp-Phe-OH |
| 117 | Ac-Phe-Orn-Ser-cha-Trp-Phe-OH |
| 118 | Ac-Phe-Orn-Pro-chg-Trp-Phe-OH |
| 119 | Ac-Phe-Eec-Pro-cha-Bta-Phe-NH₂ |
| 120 | Ac-Phe-Nle-Pro-cha-Bta-Phe-NH₂ |
| 121 | Ac-Phe-Har-Pro-cha-Bta-Phe-NH₂ |
| 122 | Ac-Phe-Arg-Pro-cha-Bta-Phe-NH₂ |
| 123 | Ac-Phe-Cys(Acm)-Pro-cha-Bta-Phe-NH₂ |
| 124 | Ac-Phe-Mpa-Pro-cha-Bta-Phe-NH₂ |
| 125 | Ac-Eby-Orn-Pro-cha-Bta-Phe-NH₂ |
| 126 | Ac-Phg-Orn-Pro-cha-Bta-Phe-NH₂ |
| 127 | Ac-Phe-Paf-Pro-cha-Bta-Phe-NH₂ |
| 128 | H₂N—CO-Phe-Orn-Pro-cha-Bta-Phe-NH₂ |
| 129 | Me—O—CO-Phe-Orn-Pro-cha-Bta-Phe-NH₂ |
| 130 | (—CO—CH₂—NH—CO—)-Phe-Orn-Pro-cha-Bta-Phe-NH₂ |
| 132 | Ac-Phe-Orn-Pro-hch-Trp-Phe-OH |
| 133 | (—CO—CH₂—CH₂—CO—)-Phe-Orn-Pro-cha-Bta-Phe-NH₂ |
| 134 | ^{t}Bu-CO-Phe-Orn-Pro-cha-Bta-Phe-NH₂ |
| 135 | Ac-Lys-Phe-Orn-Aze-cha-Bta-Phe-NH₂ |
| 136 | Ac-Gly-Phe-Orn-Aze-cha-Bta-Phe-NH₂ |
| 137 | Ac-Arg-Phe-Orn-Aze-cha-Bta-Phe-NH₂ |
| 138 | Ac-His-Phe-Orn-Aze-cha-Bta-Phe-NH₂ |
| 139 | Ac-Ser-Phe-Orn-Aze-cha-Bta-Phe-NH₂ |
| 140 | Ac-Guf-Phe-Orn-Aze-cha-Bta-Phe-NH₂ |
| 141 | Ac-Dab-Phe-Orn-Aze-cha-Bta-Phe-NH₂ |
| 142 | FH₂C—CO-Phe-Orn-Pro-cha-Bta-Phe-NH₂ |
| 143 | Ac-Phe-Orn(Et₂)-Pro-cha-Trp-Phe-NH₂ |
| 144 | Ac-Phe-[Orn-Hyp-cha-Trp-Nle] |
| 145 | 3PP-[Orn-Hyp-cha-Trp-Nle] |
| 146 | Ac-Phe-[Orn-Pro-cha-Trp-Tyr] |
| 147 | Ac-Phe-[Orn-Pro-omf-Trp-Nle] |
| 149 | Ac-Phe-Orn-Pro-hle-Bta-Phe-NH₂ |
| 150 | Ac-Phe-Arg(CH₂—CH₂)-Pro-cha-Bta-Phe-NH₂ |
| 151 | Ac-Ala-Phe-Orn-Aze-cha-Bta-Phe-NH2 |
| 152 | Ac-Arg-Phe-Orn-Aze-cha-Bta-Phe-NH2 |
| 153 | Ac-Cit-Phe-Orn-Aze-cha-Bta-Phe-NH2 |
| 154 | Ac-Gly-Phe-Orn-Aze-cha-Bta-Phe-NH2 |
| 155 | Ac-Gly-Phe-Orn-Aze-chg-Bta-Phe-NH2 |
| 156 | Ac-Gly-Phe-Orn-Aze-hch-Bta-Phe-NH2 |
| 157 | Ac-Gly-Thi-Orn-Aze-cha-Bta-Phe-NH2 |
| 158 | Ac-His-Phe-Orn-Aze-cha-Bta-Phe-NH2 |
| 159 | Ac-Hyp-Phe-Orn-Aze-cha-Bta-Phe-NH2 |
| 160 | Ac-Lys-Phe-Orn-Aze-cha-Bta-Phe-NH2 |
| 161 | Ac-Mff-Orn-Pro-cha-Bta-Phe-NH2 |
| 162 | Ac-Mff-Orn-Pro-hle-Bta-Phe-NH2 |
| 163 | Ac-Mff-Orn-Pro-hle-Mcf-Mff-NH2 |
| 164 | Ac-Mmy-Orn-Pro-hle-Pff-Phe-NH2 |
| 165 | Ac-NMF-Orn-Pro-cha-Bta-Phe-NH2 |
| 166 | Ac-Off-Orn-Pro-cha-Bta-Phe-NH2 |
| 167 | Ac-Off-Orn-Pro-hle-Bta-Phe-NH2 |
| 168 | Ac-Orn-Phe-Orn-Aze-cha-Bta-Phe-NH2 |
| 169 | Ac-Pff-Orn-Pro-cha-Bta-Phe-NH2 |
| 170 | Ac-Pff-Orn-Pro-hle-Bta-Phe-NH2 |
| 171 | Ac-Pff-Orn-Pro-hle-Mcf-Pff-NH2 |
| 172 | Ac-Phe-[Cys-Pro-cha-Bta-Phe-Cys]-NH2 |
| 173 | Ac-Phe-[Orn-Asn-cha-Trp-Nle] |
| 174 | Ac-Phe-[Orn-Aze-cha-Trp-Nle] |
| 175 | Ac-Phe-[Orn-Chy-cha-Trp-Nle] |
| 176 | Ac-Phe-[Orn-HyA-cha-Trp-Phe] |
| 177 | Ac-Phe-[Orn-Hyp-hle-Bta-Phe] |
| 178 | Ac-Phe-[Orn-Hyp-hle-Mcf-Phe] |
| 179 | Ac-Phe-[Orn-Hyp-hle-Pff-Nle] |
| 180 | Ac-Phe-[Orn-Hyp-hle-Pff-Phe] |
| 181 | Ac-Phe-[Orn-Hyp-hle-Trp-Phe] |
| 182 | Ac-Phe-[Orn-Hyp-Mmf-Trp-Nle] |
| 183 | Ac-Phe-[Orn-Hyp-Mmf-Trp-Phe] |
| 184 | Ac-Phe-[Orn-NMD-cha-Trp-Nle] |
| 185 | Ac-Phe-[Orn-Pip-hle-Bta-Phe] |
| 186 | Ac-Phe-[Orn-Pro-cha-Pff-Nle] |
| 187 | Ac-Phe-[Orn-Pro-cha-Pff-Phe] |
| 188 | Ac-Phe-[Orn-Pro-cha-Trp-1Ni] |
| 189 | Ac-Phe-[Orn-Pro-cha-Trp-Cha] |
| 190 | Ac-Phe-[Orn-Pro-cha-Trp-Chg] |
| 192 | Ac-Phe-[Orn-Pro-cha-Trp-Ecr] |
| 193 | Ac-Phe-[Orn-Pro-cha-Trp-Leu] |
| 194 | Ac-Phe-[Orn-Pro-cha-Trp-nle] |
| 195 | Ac-Phe-[Orn-Pro-cha-Trp-Phe] |
| 196 | Ac-Phe-[Orn-Pro-hle-Bta-Nle] |
| 197 | Ac-Phe-[Orn-Pro-hle-Bta-Phe] |
| 198 | Ac-Phe-[Orn-Pro-hle-Pff-Phe] |
| 199 | Ac-Phe-[Orn-Pro-hle-Trp-Nle] |
| 200 | Ac-Phe-[Orn-Ser-cha-Trp-Nle] |
| 201 | Ac-Phe-[Orn-Ser-cha-Trp-Nle] |
| 202 | Ac-Phe-[Orn-Ser-hle-Trp-Nle] |
| 203 | Ac-Phe-[Orn-Thr-cha-Trp-Nle] |
| 204 | Ac-Phe-[Orn-Tic-cha-Trp-Nle] |
| 205 | Ac-Phe-[Orn-Tic-cha-Trp-Nle] |
| 206 | Ac-Phe-Ala-Pro-cha-Bta-Phe-NH2 |
| 207 | Ac-Phe-Arg-Pro-hle-Bta-Phe-NH2 |
| 208 | Ac-Phe-Arg-Pro-hle-Mcf-Phe-NH2 |
| 209 | Ac-Phe-Cit-Hyp-hle-Bta-Phe-NH2 |
| 210 | Ac-Phe-Cit-Pro-cha-Bta-Phe-NH2 |
| 211 | Ac-Phe-Cit-Pro-hle-Bta-Phe-NH2 |
| 212 | Ac-Phe-Cit-Ser-hle-Bta-Phe-NH2 |
| 213 | Ac-Phe-Dab-Aze-cha-Bta-Phe-NH2 |
| 214 | Ac-Phe-Dab-Aze-hle-Bta-Phe-NH2 |
| 215 | Ac-Phe-Dab-Pro-cha-Bta-Phe-NH2 |
| 216 | Ac-Phe-Dap-Pro-cha-Bta-Phe-NH2 |
| 217 | Ac-Phe-Ech-Pro-cha-Bta-Phe-NH2 |
| 218 | Ac-Phe-Eep-Pro-cha-Bta-Phe-NH2 |
| 219 | Ac-Phe-Fcn-Aze-cha-Bta-Phe-NH2 |
| 220 | Ac-Phe-Fcn-Pro-cha-Bta-Phe-NH2 |
| 221 | Ac-Phe-Fco-Pro-cha-Bta-Phe-NH2 |
| 222 | Ac-Phe-Fco-Pro-cha-Bta-Phe-NH2 |
| 223 | Ac-Phe-Fcp-Aze-cha-Bta-Phe-NH2 |
| 224 | Ac-Phe-Ffa-Aze-cha-Bta-Phe-NH2 |
| 225 | Ac-Phe-Ffa-Pro-cha-Bta-Phe-NH2 |
| 226 | Ac-Phe-Ffa-Pro-hle-Bta-Phe-NH2 |
| 227 | Ac-Phe-G23-Pro-cha-Bta-Phe-NH2 |
| 228 | Ac-Phe-Guf-Pro-cha-Bta-Phe-NH2 |
| 229 | Ac-Phe-Har-Aze-cha-Bta-Phe-NH2 |
| 230 | Ac-Phe-His-Pro-cha-Bta-Phe-NH2 |
| 231 | Ac-Phe-L22-Pro-cha-Bta-Phe-NH2 |
| 232 | Ac-Phe-OrA-Pro-cha-Bta-Phe-NH2 |
| 233 | Ac-Phe-OrE-Pro-cha-Bta-Phe-NH2 |
| 234 | Ac-Phe-Orn-Aze-hle-Bta-Phe-NH2 |
| 235 | Ac-Phe-Orn-Chy-cha-Bta-Phe-NH2 |
| 236 | Ac-Phe-Orn-Chy-hle-Pff-Phe-NH2 |
| 237 | Ac-Phe-Orn-G24-cha-Bta-Phe-NH2 |
| 238 | Ac-Phe-Orn-G25-cha-Bta-Phe-NH2 |
| 239 | Ac-Phe-Orn-G26-cha-Bta-Phe-NH2 |
| 240 | Ac-Phe-Orn-G27-cha-Bta-Phe-NH2 |
| 241 | Ac-Phe-Orn-G30-cha-Bta-Phe-NH2 |
| 242 | Ac-Phe-Orn-G31-cha-Bta-Phe-NH2 |
| 243 | Ac-Phe-Orn-Hse-cha-Bta-Phe-NH2 |
| 244 | Ac-Phe-Orn-Hyp-hle-Bta-Phe-NH2 |
| 245 | Ac-Phe-Orn-Hyp-hle-Pff-Phe-NH2 |
| 246 | Ac-Phe-Orn-NMA-cha-Bta-Phe-NH2 |
| 247 | Ac-Phe-Orn-NMS-cha-Bta-Phe-NH2 |
| 248 | Ac-Phe-Orn-Pro-cha-1Ni-Phe-NH2 |
| 249 | Ac-Phe-Orn-Pro-cha-Bta-1Ni—NH2 |
| 250 | Ac-Phe-Orn-Pro-cha-Bta-Bhf-NH2 |
| 251 | Ac-Phe-Orn-Pro-cha-Bta-Dff-NH2 |
| 252 | Ac-Phe-Orn-Pro-cha-Bta-Eaa-NH2 |
| 253 | Ac-Phe-Orn-Pro-cha-Bta-L19 |
| 254 | Ac-Phe-Orn-Pro-cha-Bta-Mcf-NH2 |
| 255 | Ac-Phe-Orn-Pro-cha-Bta-Mff-NH2 |
| 256 | Ac-Phe-Orn-Pro-cha-Bta-NH—CH(CH2OH)—CH2-Ph |
| 257 | Ac-Phe-Orn-Pro-Cha-Bta-NH-NBn-CO—NH2 |
| 258 | Ac-Phe-Orn-Pro-cha-Bta-Opa-NH2 |
| 259 | Ac-Phe-Orn-Pro-cha-Bta-Pcf-NH2 |
| 260 | Ac-Phe-Orn-Pro-cha-Bta-Pmf-NH2 |
| 261 | Ac-Phe-Orn-Pro-cha-Bta-Thi-NH2 |
| 262 | Ac-Phe-Orn-Pro-cha-Otf-Phe-NH2 |
| 263 | Ac-Phe-Orn-Pro-ctb-Bta-Phe-NH2 |
| 264 | Ac-Phe-Orn-Pro-ctb-Eaa-Phe-NH2 |
| 265 | Ac-Phe-Orn-Pro-ctb-Mcf-Phe-NH2 |
| 266 | Ac-Phe-Orn-Pro-ctb-Pff-Phe-NH2 |
| 267 | Ac-Phe-Orn-Pro-hch-Trp-Phe-OH |
| 268 | Ac-Phe-Orn-Pro-hle-1Ni-Phe-NH2 |
| 269 | Ac-Phe-Orn-Pro-hle-6FW-Phe-NH2 |
| 270 | Ac-Phe-Orn-Pro-hle-Bta-1Ni—NH2 |
| 271 | Ac-Phe-Orn-Pro-hle-Bta-2Ni—NH2 |
| 272 | Ac-Phe-Orn-Pro-hle-Bta-5Ff-NH2 |
| 273 | Ac-Phe-Orn-Pro-hle-Bta-Aic-NH2 |
| 274 | Ac-Phe-Orn-Pro-hle-Bta-Cha-NH2 |
| 275 | Ac-Phe-Orn-Pro-hle-Bta-Chg-NH2 |
| 276 | Ac-Phe-Orn-Pro-hle-Bta-Eaa-NH2 |
| 277 | Ac-Phe-Orn-Pro-hle-Bta-Egy-NH2 |
| 278 | Ac-Phe-Orn-Pro-hle-Bta-Pcf-NH2 |
| 279 | Ac-Phe-Orn-Pro-hle-Bta-Pff-NH2 |
| 280 | Ac-Phe-Orn-Pro-hle-Bta-Phe-NH2 |
| 281 | Ac-Phe-Orn-Pro-hle-Bta-phe-OH |
| 282 | Ac-Phe-Orn-Pro-hle-Bta-Tyr-NH2 |
| 283 | Ac-Phe-Orn-Pro-hle-Dff-Phe-NH2 |
| 284 | Ac-Phe-Orn-Pro-hle-Eaa-Phe-NH2 |
| 285 | Ac-Phe-Orn-Pro-hle-Egc-Phe-NH2 |
| 286 | Ac-Phe-Orn-Pro-hle-Egy-Phe-NH2 |
| 287 | Ac-Phe-Orn-Pro-hle-Egz-Phe-NH2 |
| 288 | Ac-Phe-Orn-Pro-hle-Mcf-2Ni—NH2 |
| 289 | Ac-Phe-Orn-Pro-hle-Mcf-Cha-NH2 |
| 290 | Ac-Phe-Orn-Pro-hle-Mcf-Pff-NH2 |
| 291 | Ac-Phe-Orn-Pro-hle-Mcf-Phe-NH2 |
| 292 | Ac-Phe-Orn-Pro-hle-Mff-Phe-NH2 |
| 293 | Ac-Phe-Orn-Pro-hle-Mmy-Phe-NH2 |
| 294 | Ac-Phe-Orn-Pro-hle-Ocf-Phe-NH2 |
| 295 | Ac-Phe-Orn-Pro-hle-Off-Phe-NH2 |
| 296 | Ac-Phe-Orn-Pro-hle-Otf-Phe-NH2 |
| 297 | Ac-Phe-Orn-Pro-hle-Pff-2Ni—NH2 |
| 298 | Ac-Phe-Orn-Pro-hle-Pff-Cha-NH2 |
| 299 | Ac-Phe-Orn-Pro-hle-Pff-Eaa-NH2 |
| 300 | Ac-Phe-Orn-Pro-hle-Pff-Mmy-NH2 |
| 301 | Ac-Phe-Orn-Pro-hle-Pff-Pff-NH2 |
| 302 | Ac-Phe-Orn-Pro-hle-Pff-Phe-NH2 |
| 304 | Ac-Phe-Orn-Pro-hle-Phe-Phe-NH2 |
| 305 | Ac-Phe-Orn-Pro-hle-Tff-Phe-NH2 |
| 306 | Ac-Phe-Orn-Pro-hle-Trp-Phe-NH2 |
| 307 | Ac-Phe-Orn-Pro-ile-Trp-Phe-NH2 |
| 308 | Ac-Phe-Orn-Pro-omf-Bta-Phe-NH2 |
| 309 | Ac-Phe-Orn-Ser-cha-Bta-Phe-NH2 |
| 310 | Ac-Ser-Phe-Orn-Aze-cha-Bta-Phe-NH2 |
| 311 | Ac-Thi-[Orn-Pro-hle-Bta-Phe] |
| 312 | Ac-Thi-Orn-Pro-cha-Bta-Phe-NH2 |
| 313 | Ac-Thi-Orn-Pro-cha-Bta-Thi-NH2 |
| 314 | Ac-Thr-Phe-Orn-Aze-cha-Bta-Phe-NH2 |
| 315 | Bzl-[Orn-Pro-cha-Bta-Nle] |
| 316 | CH3CH2CO-Phe-Orn-Pro-cha-Bta-Phe-NH2 |
| 317 | Def-[Orn-Ser-hle-Trp-Nle] |
| 318 | Eby-Phe-[Orn-Hyp-cha-Trp-Phe] |
| 319 | Eth-Phe-[Orn-Pro-hle-Pff-Nle] |
| 320 | FAc-Phe-Fib-Aze-cha-Bta-Phe-NH2 |
| 321 | FAc-Phe-Orn-Aze-cha-Bta-Phe-NH2 |
| 322 | FAc-Phe-Orn-Pro-cha-Bta-Phe-NH2 |
| 323 | Fai-Phe-[Orn-Hyp-cha-Trp-Phe] |
| 324 | Faz-Orn-Pro-cha-Bta-Phe-NH2 |
| 325 | Fbi-Phe-[Orn-Pro-cha-Trp-Nle] |
| 326 | Fbn-Phe-[Orn-Hyp-cha-Trp-Phe] |
| 327 | Fbn-Phe-[Orn-Pro-cha-Trp-Nle] |
| 328 | Fbn-Phe-[Orn-Pro-cha-Trp-Nle] |
| 329 | Fbn-Phe-Cit-Pro-hle-Bta-Phe-NH2 |
| 330 | Fbo-Phe-[Orn-Pro-cha-Trp-Nle] |
| 331 | Fbp-[Orn-Pro-cha-Trp-Nle] |
| 332 | Fci-[Phe-Orn-Hyp-cha-Trp-Phe] |
| 333 | Fck-[Phe-Orn-Pro-cha-Trp-Nle] |
| 334 | Fck-Phe-[Orn-Pro-cha-Trp-Nle] |
| 335 | Fha-Phe-[Orn-Hyp-cha-Trp-Phe] |
| 336 | Fhb-[Phe-Orn-Hyp-cha-Trp-Phe] |
| 337 | Fhi-Phe-[Orn-Hyp-cha-Trp-Phe] |
| 338 | Fhu-Phe-[Orn-Pro-hle-Pff-Nle] |
| 339 | Fhu-Phe-Orn-Pro-cha-Bta-Phe-NH2 |
| 340 | Fid-Phe-Orn-Pro-cha-Bta-Phe-NH2 |
| 341 | H-Amf-[Orn-Aze-hle-Pff-Nle] |
| 342 | H-Bal-Phe-[Orn-Hyp-hle-Trp-Nle] |
| 343 | H-Bal-Phe-[Orn-Pro-hle-Pff-Nle] |
| 344 | H-Eby-[Orn-Hyp-hle-Trp-Nle] |
| 345 | H-Gly-Phe-Orn-Pro-cha-Bta-Phe-NH2 |
| 346 | H-Nip-Phe-Cit-Pro-hle-Bta-Phe-NH2 |
| 347 | Hoo-Phe-[Orn-Hyp-hle-Pff-Nle] |
| 348 | Hoo-Phe-Cit-Pro-hle-Pff-Phe-NH2 |
| 349 | Hoo-Phe-Orn-Hyp-hle-Pff-Phe-NH2 |
| 350 | Hoo-Phe-Orn-Pro-hle-Bta-Phe-NH2 |
| 351 | Hoo-Phe-Orn-Pro-hle-Mcf-Phe-NH2 |
| 352 | Hoo-Phe-Orn-Pro-hle-Pff-Phe-NH2 |
| 353 | H-Phe-[Lys-Hyp-hle-Pff-Nle] |
| 354 | H-Phe-[Orn-Hym-hle-Mcf-Nle] |
| 355 | H-Phe-[Orn-Hym-hle-Pff-Phe] |
| 356 | H-Phe-[Orn-Hyp-cha-Trp-Nle] |
| 357 | H-Phe-[Orn-Hyp-cha-Trp-Phe] |
| 358 | H-Phe-[Orn-Hyp-ctb-Pff-Nle] |
| 359 | H-Phe-[Orn-Hyp-ctb-Trp-Nle] |
| 360 | H-Phe-[Orn-Hyp-ctb-Trp-Phe] |
| 361 | H-Phe-[Orn-Hyp-hle-Mcf-Leu] |
| 362 | H-Phe-[Orn-Hyp-hle-Pff-Chg] |
| 363 | H-Phe-[Orn-Hyp-hle-Pff-Hle] |
| 364 | H-Phe-[Orn-Hyp-hle-Pff-Leu] |
| 365 | H-Phe-[Orn-Hyp-hle-Pff-Nle] |
| 366 | H-Phe-[Orn-Hyp-hle-Pff-Phe] |
| 367 | H-Phe-[Orn-Hyp-hle-Trp-Hle] |
| 368 | H-Phe-[Orn-Hyp-hle-Trp-Leu] |
| 369 | H-Phe-[Orn-Hyp-hle-Trp-Nle] |
| 370 | H-Phe-[Orn-Hyp-hle-Trp-Nva] |
| 371 | H-Phe-[Orn-Hyp-hle-Trp-Phe] |
| 372 | H-Phe-[Orn-NMS-cha-Trp-Nle] |
| 373 | H-Phe-[Orn-NMS-hle-Pff-Phe] |
| 374 | H-Phe-[Orn-Pro-cha-Pff-Nle] |
| 375 | H-Phe-[Orn-Pro-cha-Pff-Phe] |
| 376 | H-Phe-[Orn-Pro-cha-Trp-Nle] |
| 377 | H-Phe-[Orn-Pro-hle-Mcf-Phe] |
| 378 | H-Phe-[Orn-Pro-hle-Ocf-Phe] |
| 379 | H-Phe-[Orn-Pro-hle-Pff-Nle] |
| 380 | H-Phe-[Orn-Pro-hle-Pff-Phe] |
| 381 | H-Phe-[Orn-Pro-hle-Trp-Nle] |
| 382 | H-Phe-[Orn-Ser-cha-Trp-Nle] |
| 383 | H-Phe-[Orn-Ser-cha-Trp-Phe] |
| 384 | H-Phe-[Orn-Ser-hle-Eaa-Nle] |
| 385 | H-Phe-[Orn-Ser-hle-Mcf-Leu] |
| 386 | H-Phe-[Orn-Ser-hle-Ocf-Nle] |
| 387 | H-Phe-[Orn-Ser-hle-Pff-Leu] |
| 388 | H-Phe-[Orn-Ser-hle-Pff-Nle] |
| 389 | H-Phe-[Orn-Ser-hle-Pff-Phe] |
| 390 | H-Phe-[Orn-Ser-hle-Trp-Nle] |
| 391 | H-Phe-Cit-Pro-hle-Bta-Phe-NH2 |
| 392 | Ohf-[Orn-Hyp-hle-Trp-Nle] |
| 393 | Tmg-Phe-[Orn-Hyp-cha-Trp-Phe] |

45. A pharmaceutical composition comprising at least one compound according to claim 1 and a pharmaceutically acceptable carrier.

46. Use of at least one of the compounds according to claim 1 for the manufacture of a medicament.

47. Use according to claim 46, whereby the medicament is used or useful for the prevention and/or treatment of a condition associated with complement activation and/or a condition where the inhibition of the complement system leads to a relief of the symptoms.

48. Use according to claim 46, whereby the medicament is used or useful for the prevention and/or treatment of a condition where the inhibition of the C5a receptor alone or in combination with other therapeutics leads to a relief of the symptoms.

49. Use according to claim 46, whereby the condition and/or the symptoms to be treated are selected from the group comprising autoimmune diseases, acute inflammatory diseases, trauma, local inflammations, shock and burn.

50. Use according to claim 46, whereby the condition is selected from the group comprising sarcoidosis, septic shock, haemorrhagic shock, systemic inflammatory response syndrome (SIRS), multiple organ failure (MOF), asthma, vasculitis, myocarditis, dermatomyositis, inflammatory bowel disease (IBD), pemphigus, glomerulonephritis, acute respiratory insufficiency, stroke, myocardial infarction, reperfusion injury, neurocognitive dysfunction, burn, inflammatory diseases of the eye, local manifestations of systemic diseases, inflammatory diseases of the vessels, and acute injuries of the central nervous system.

51. Use according to claim 50, whereby the inflammatory disease of the eye is selected from the group comprising uveitis, age-related macular degeneration, diabetic retinopathy, diabetic macular edema, ocular pemphigoid, keratoconjunctivitis, Stevens-Johnson syndrome, and Graves opthalmopathy.

52. Use according to claim 50, whereby the condition is a local manifestation of a systemic disease, whereby the systemic disease is selected from the group comprising rheumatoid arthritis, SLE, type I diabetes, and type II diabetes.

53. Use according to claim 52, whereby the manifestations are selected from the group comprising manifestations at the eye, at or in the brain, at the vessels, at the heart, at the lung, at the kidneys, at the liver, at the gastro-intestinal tract, at the spleen, at the skin, at the skeletal system, in the lymphatic system, and in the blood.

54. Use according to claim 49, whereby the autoimmune disease is selected from the group comprising alopecia greata, cold agglutinin immunohemolytic anemia, warm antibody immunohemolytic anemia, pernicious anemia (Biermer's Disease, Addison's anemia), antiphospholipid antibody syndrome (APS), arteriitis temporalis, atherosclerosis, autoimmune adrenalitis (Addison's disease), chronic fatigue syndrome (CFIDS), chronic-inflammatory polyneuropathy, Churg-Strauss syndrome, Cogan's syndrome, ulcerative colitis, CREST syndrome, diabetes mellitus type I, dermatitis herpetiformis, dermatomyositis, fibromyalgia, chronic autoimmune gastritis, Goodpasture syndrome (anti-GBM antibody related glomerulonephritis), Guillain-Barré syndrome (GBS; Polyradiculoneuropathy), Hashimoto thyroiditis, autoimmune hepatitis, idiopathic pulmonary fibrosis, autoimmune thrombocytopenice purpura (Werlhof's Disease), autoimmune infertility, autoimmune inner ear deafness (AIED), juvenile rheumatoid arthritis, autoimmune cardiomyopathy, Lambert-Eaton syndrome, Lichen sclerosus, Lupus erythematosus, Lyme arthritis, collagenosis, Graves' disease, Behcet disease, Crohn's disease, rheumatoid spondylitis, Méniére's disease, Reiter's disease, multiple sclerosis (MS, encephalomyelitis), myasthenia gravis, sympathetic ophtalmy, scaring pemphigoid, bullous pemphigoid, pemphigus vulgaris, polyarteritis nodosa, polychondritis, polyglandular autoimmune (PGA) syndrome, polymyalgia rheumatica, polymyositis, primary biliary cirrhosis, psoriasis, rheumatic fever, rheumatoid arthritis, sarcoidosis (Besnier-Boeck-Schaumann's disease), Sjörgen syndrome, scleroderma, sprue, stiff-man syndrome, systemic lupus erythematosus, Takayasu arteritis, transiente gluten intolerance, autoimmune uveitis, vasculites and vitiligo

55. Use according to claim 50, in which the inflammatory diseases of the vessels are selected from the group comprising vasculitis, vascular leakage, and atherosclerosis.

56. Use according to claim 55, whereby the vasculitis is selected from the group comprising primary and secondary vasculites.

57. Use according to claim 56, whereby the primary vasculitis is selected from the group comprising Wegener's disease, Churg Strauss syndrome and microscopic polyangitis.

58. Use according to claim 56, whereby the secondary vasculitis is selected from the group of vasculites induced through drug use and vasculitis induced by other diseases.

59. Use according to claim 58, whereby the disease is selected from the group comprising AIDS, hepatitis B, hepatitis C and cytomegalovirus infection.

60. Use according to claim 46, whereby the medicament is used to influence the adaptive and the innate immune system or useful for such a purpose.

61. Use according to claim 60, whereby the influence is a strengthening of the immune system.

62. Use according to claim 46, whereby the medicament is for the prevention and/or support of surgical manipulations.

63. Use according to claim 62 whereby the surgical manipulation is selected from the group comprising CABG, PACT, PTA, MidCAB, OPCAB, thrombolysis, organ transplantation, and vessel clamping.

64. Use according to claim 46, hereby the medicament is used or useful for thrombolytic treatment.

65. Use according to claim 46, whereby the medicament is used or useful in the settings of dialysis before, during, and/or after the treatment, preferably dialysis treatment.

66. Use according to claim 46, whereby the medicament is used or useful for the prevention of organ damage of a transplanted organ or of an organ that will be transplanted.

67. Use according to claim 46, whereby the medicament is used or useful for the prevention or treatment of transplant rejection.
